(19) United States
(12) Patent Application Publication
(10) Pub. No.: US 2005/0239806 A1
(43) Pub. Date: Oct. 27, 2005

(54) Pyrrolopyrimidine derivatives and analogs and their use in the treatment and prevention of diseases

(75) Inventors: Shamal A. Mehta, San Diego, CA (US); Andiliy G. Sai, San Diego, CA (US); Zdravko V. Milanov, San Diego, CA (US); Robert M. Grotzfeld, Carlsbad, CA (US); Hitesh K. Patel, Encinitas, CA (US); David J. Lockhart, Del Mar, CA (US)

Correspondence Address: WILSON SONSINI GOODRICH & ROSATI, 650 PAGE MILL ROAD, PALO ALTO, CA, 94304-1050, US

(73) Assignee: Ambit Biosciences Corporation

(21) Appl. No.: 11/035,619

(22) Filed: Jan. 13, 2005

Related U.S. Application Data
(60) Provisional application No. 60/536,301, filed on Jan. 13, 2004.
(60) Provisional application No. 60/602,460, filed on Aug. 18, 2004.
(60) Provisional application No. 60/602,584, filed on Aug. 18, 2004.
(60) Provisional application No. 60/602,586, filed on Aug. 18, 2004.

(51) Int. Cl. A61K 31/519; A61K 31/522

(52) U.S. Cl. 514/260.1; 514/263.3; 514/263.34; 514/263.37; 514/265.1

(57) ABSTRACT

Described herein are compounds and compositions for modulating kinase activity, and methods for modulating kinase activity using the compounds and compositions. Also described herein are methods of using the compounds and/or compositions in the treatment and prevention of a variety of diseases and unwanted conditions in subjects.

### CROSS REFERENCE TO RELATED APPLICATIONS

This application claims the benefit of U.S. Provisional Application No. 60/536,301 filed Jan. 13, 2004, U.S. Provisional Application No. 60/602,460 filed Aug. 18, 2004, U.S. Provisional Application No. 60/602,584 filed Aug. 18, 2004, and U.S. Provisional Application No. 60/602,586 filed Aug. 18, 2004, the disclosures of each of which are incorporated herein by reference in their entirety.

### BACKGROUND OF THE INVENTION

The protein kinases (PKs) are enzymes that catalyze the phosphorylation of hydroxy groups on tyrosine, serine and threonine residues of proteins. The PKs are categorized into two classes: the protein tyrosine kinases (PTKs) and the serine-threonine kinases (STKs). The activity of PTKs is primarily associated with growth factor receptors. Growth factor receptors are cell-surface proteins that are converted to an active form upon the binding of a growth factor ligand. The active form interacts with proteins on the inner surface of a cell membrane leading to phosphorylation on tyrosine residues of the receptor and other proteins (Schlessinger and Ullrich (1992) Neuron 9:303-391). The serine-threonine kinases (STKs) are predominantly intracellular, and are the most common of the cytosolic kinases. The protein kinases have been implicated in a host of pathogenic conditions including, cancer, psoriasis, hepatic cirrhosis, diabetes, angiogenesis, restenosis, ocular diseases, rheumatoid arthritis and other inflammatory disorders, immunological disorders such as autoimmune disease, cardiovascular disease such as atherosclerosis and a variety of renal disorders.

Growth factor receptors with PTK activity are known as receptor tyrosine kinases (RTKs). At present, at least nineteen (19) distinct subfamilies of RTKs have been identified, including the “HER” subfamily which includes EGFR (epidermal growth factor receptor), HER2, HER3 and HER4. These RTKs consist of an extracellular glycosylated ligand binding domain, a transmembrane domain and an intracellular cytoplasm catalytic domain that can phosphorylate tyrosine residues on proteins. Other RTK subfamily consists of insulin receptor (IR); insulin-like growth factor I receptor (IGF-1R); insulin receptor related receptor (IRR); the platelet derived growth factor receptor (PDGFR) group, which includes PDGFR-α, PDGFR-β, CSFIR, c-kit and c-fins; the fetus liver kinase (flk) receptor subfamily which includes fetal liver kinase-1 (KDR/FLK-1, VEGFR-2), flk-1R, flk-4 and fins-like tyrosine kinase 1 (flt-1); the tyrosine kinase growth factor receptor family is the fibroblast growth factor (FGF) receptor subgroup; and the vascular endothelial growth factor (VEGF) receptor subgroup. In addition to the RTKs, there also exists a family of intracellular PTKs called “non-receptor tyrosine kinases” or “cellular tyrosine kinases” (CTK). At present, over 24 CTKs in 11 subfamilies (Src, Frk, Btk, Csk, Abll, Zap70, Fes, Fps, Fak, Jak and Ack) have been identified. The Src subfamily is the largest group and includes Src, Yes, Fyn, Lyn, Lck, Blk, Hck, Fgr and Yrk (Bolen (1993) Oncogene, 8:2025-2031).

One class of compounds known to inhibit certain tyrosine kinases include pyrimidine compounds. For example, U.S. Pat. No. 6,635,762 to Blumenkopf et al. describes pyrrolo[2,3-d]pyrimidine compounds. The compounds can be used to inhibit protein tyrosine kinases, especially Janus Kinase 3 (JAK3). U.S. Pat. No. 6,627,754 to Blumenkopf et al. describes 4-aminopyrrolo[2,3-d]pyrimidine compounds, where the amine is at least a secondary amine, and use of the compounds to inhibit protein tyrosine kinases, especially Janus Kinase 3 (JAK3). The patent also discloses use of the compounds for treating diseases such as diabetes, cancer, autoimmune diseases, and the like.

Various pyrimidine compounds have also been identified as inhibitors of EGFR. U.S. Pat. No. 6,395,733 to Arnold et al. describes 4-aminopyrrolo[2,3-d]pyrimidine compounds. The compounds are also said to inhibit EGFR. U.S. Pat. No. 6,251,911 to Bold et al. describes 4-amino-1H-pyrazolo[3,4-d]pyrimidine compounds having EGFR and c-erb B2 activity. U.S. Pat. No. 6,140,317 to Traxler et al. describes 4-substituted pyrrolo[2,3-d]pyridmidine compounds, and U.S. Pat. Nos. 6,140,332, 6,096,749, and 5,686,457, all to Traxler et al. describes 4-aminopyrrolo[2,3-d]pyrimidine compounds, 4-aniline pyrrolo[2,3-d]pyrimidine compounds, and 4-aniline pyrrolo[2,3-d]pyrimidine compounds respectively. The compounds are said to inhibit EGFR.

U.S. Pat. No. 6,207,669 to Cockerill et al. describes substituted bicyclic heteroaromatic compounds and their use as inhibitors of protein tyrosine kinase activity, such as EGFR.

### SUMMARY OF THE INVENTION

Provided herein are methods and compositions for treating and/or preventing conditions and diseases associated with kinase activity, e.g., EGFR activity, such as cancer, hyperplasia, psoriasis, cardiac hypertrophy, arthrosclerosis, dermatitis and/or diseases or conditions associated with undesired cellular hyperproliferation. In particular, hetercyclic compounds that preferentially inhibit one or more of the EGFR protein tyrosine kinases, e.g., EGFR (HER 1, erbB1), erbB2 (HER2, c-Neu), erbB3 (HER3) and erbB4 (HER4). In some embodiments, the compounds modulate protein kinase activity. In other embodiments, the compounds modulate receptor tyrosine kinases.

The compounds described herein can be delivered alone or in combination with additional agents, and are used for the treatment and/or prevention of conditions and diseases. Thus, the compounds are useful in treating disorders mediated by EGFR tyrosine kinases and in particular have anti-proliferative properties. In some embodiments, the compounds and compositions are used for the prevention or treatment of cancers such as stomach, gastric, bone, ovary, colon, lung, brain, larynx, lymphatic system, genitourinary tract, ovarian, squamous cell carcinoma, astrocytoma, Kaposi's sarcoma, glioblastoma, lung cancer, bladder cancer, head and neck cancer, melanoma, ovarian cancer, prostate cancer, breast cancer, lung cancer, leukemia, glioma, colorectal cancer, genitourinary cancer, gastrointestinal cancer, or pancreatic cancer.

In one aspect, methods for preventing further progression of the conditions or diseases, or, optionally for treating and/or preventing such conditions and diseases in a subject in need thereof are provided.

Provided herein are compositions, methods of treating, and methods for modulating the activity of epidermal growth factor receptor comprising providing an effective amount of a compound of Formula Ia, Formula Iaa, Formula Ib or Formula Ibb: 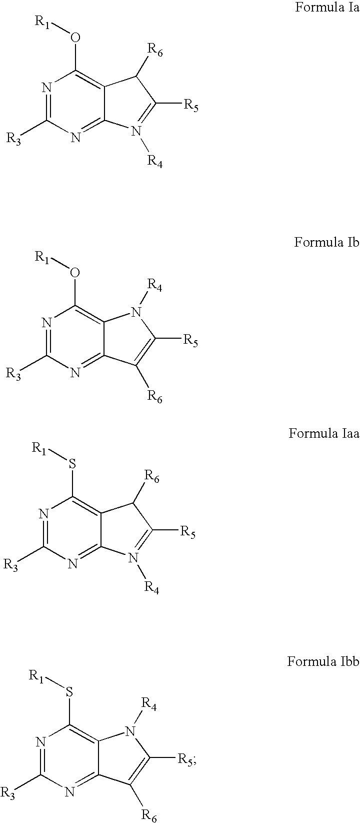
wherein
(a) R₁ is selected from one of the following options:
   a. R₁ is a moiety having the structure —(CHR₁ₐ)_{z}—R_{1b},
      i. wherein z is a number selected from the group consisting of 0, 1, 2, 3 and 4;
      ii. R₁ₐ is a moiety selected from the group consisting of H, (C₁-C₄)alkyl, F, (C₁-C₄)fluoroalkyl, (C₁-C₄)alkoxy, —C(O)OH, —C(O)—NH₂, —C(O)—(C₁-C₄)alkyl, —C(O)—(C₁-C₄)fluoralkyl, —C(O)—(C₁-C₄)alkylamine, and —C(O)—(C₁-C₄)alkoxy;
      iii. R_{1b} is phenyl, optionally substituted with 1-4 moieties independently selected from the group consisting of halogen, —CN, -L₁-OH, -L₁-NH₂, -L₁-(C₁-C₄)alkyl, -L₁-(C₃-C₆)cycloalkyl, -L₁-(C₁-C₄)fluoroalkyl, -L₁-(C₁-C₄)alkoxy, -L₁-(C₁-C₄)alkylamine, -L₁-(C₁-C₄)dialkylamine and -L₁-phenyl, wherein L₁ is a bond, —C(O)— and S(O)₂; or
   b. R₁ is a moiety having the structure —(CHR₁ₐ)_{z}—R_{1b},
      i. wherein z is a number selected from the group consisting of 0, 1, 2 and 3;
      ii. R₁ₐ is a moiety selected from the group consisting of H, (C₁-C₄)alkyl, F, (C₁-C₄)fluoroalkyl, (C₁-C₄)alkoxy, —(C₁-C₄)alkylamine, —(C₁-C₄)dialkylamine, —C(O)OH, —C(O)—NH₂, —C(O)—(C₁-C₄)alkyl, —C(O)—(C₁-C₄)fluoralkyl, —C(O)—(C₁-C₄)alkylamine, and —C(O)—(C₁-C₄)alkoxy;
      iii. R_{1b} is a moiety selected from the group consisting of —(C₁-C₄)alkyl, an optionally substituted —(C₃-C₆)cycloalkyl, —(C₁-C₄)fluoroalkyl, and an optionally substituted 5-membered or 6-membered unsaturated heterocycle; or R_{1b} is H when z is 1, 2, or 3; and
(b) R₃ is L₃-(CHR₃ₐ)ₓ—R_{3b}, wherein x is 0, 1, 2, or 3; L₃ is a bond, NH, O or S; R₃ₐ is selected from the group consisting of H, (C₁-C₄)alkyl, F, (C₁-C₄)fluoroalkyl, (C₁-C₄)alkoxy, —(C₁-C₄)alkylamine, and —(C₁-C₄)dialkylamine; and R_{3b} is H or a phenyl, optionally substituted with 1-2 substituents independently selected from the group consisting of halogen, —(C₁-C₄)alkyl, —(C₁-C₄)fluoroalkyl, —(C₁-C₄)alkoxy, —(C₁-C₄)alkylamine, and —(C₁-C₄)dialkylamine;
(c) R₄ is a moiety having the structure —(CHR₄ₐ)_{y}—R_{4b},
   i. wherein y is a number selected from the group consisting of 0, 1, 2 and 3;
   ii. R₄ₐ is a moiety selected from the group consisting of H, (C₁-C₄)alkyl, F, (C₁-C₄)fluoroalkyl, (C₁-C₄)alkoxy, —(C₁-C₄)alkylamine, —(C₁-C₄)dialkylamine;
   iii. R_{4b} is a moiety selected from the group consisting of H, —(C₁-C₄)alkyl, an optionally substituted —(C₃-C₆)cycloalkyl, —(C₁-C₄)fluoroalkyl, an optionally substituted phenyl, and an optionally substituted 5-membered or 6-membered unsaturated heterocycle;
(d) R₅ is H or phenyl, optionally substituted with 1-2 moieties independently selected from the group consisting of halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, —(C₁-C₄)dialkylamine, —C(O)OH, —C(O)—NH₂, —C(O)—(C₁-C₄)alkyl, —C(O)—(C₁-C₄)fluoralkyl, —C(O)—(C₁-C₄)alkylamine, and —C(O)—(C₁-C₄)alkoxy; and
   R₆ is a moiety selected from the group consisting of H, heteroaryl, and phenyl, wherein the phenyl and the heteroaryl are optionally substituted with 1-2 moieties independently selected from the group consisting of halogen, —(C₁-C₄)alkyl, —(C₁-C₄)fluoroalkyl, —(C₁-C₄)alkoxy, —(C₁-C₄)alkylamine, and —(C₁-C₄)dialkylamine; or
   when the compound has the structure of Formula Ia or Formula Iaa, R₁ and R₆ together form a 5- or 6-membered heterocyclic aromatic ring structure, optionally substituted with 1-2 moieties independently selected from the group consisting of halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, and —(C₁-C₄)dialkylamine; or
   when the compound has the structure of Formula Ib or Formula Ibb, R₁ and R₄ together form a 5- or 6-membered heterocyclic aromatic ring structure, optionally substituted with 1-2 moieties independently selected from the group consisting of halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, and —(C₁-C₄)dialkylamine; or
   R₄ and R₅ together form a 5- or 6-membered heterocyclic aromatic ring structure, optionally substituted with 1-2 moieties independently selected from the group consisting of halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, and —(C₁-C₄)dialkylamine; or
   R₅ and R₆ together form a 5- or 6-membered carbocyclic or heterocyclic aromatic ring structure, optionally substituted with 1-2 moieties independently selected from the group consisting of halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, and —(C₁-C₄)dialkylamine;
   or a pharmaceutically acceptable salt, pharmaceutically acceptable N-oxide, pharmaceutically active metabolite, pharmaceutically acceptable prodrug, or pharmaceutically acceptable solvate thereof.

Provided herein are compositions, methods of treating, and methods for modulating the activity of epidermal growth factor receptor comprising providing an effective amount of a compound of any of Formula Ia, Formula Iaa, Formula Ib and Formula Ibb wherein R₁ is a moiety having the structure —(CHR₁ₐ)_{z}—R_{1b}, wherein z is a number selected from the group consisting of 0, 1, 2 and 3; R₁ₐ is a moiety selected from the group consisting of H, (C₁-C₄)alkyl, F, (C₁-C₄)fluoroalkyl, (C₁-C₄)alkoxy, —C(O)OH, —C(O)—NH₂, —C(O)—(C₁-C₄)alkyl, —C(O)—(C₁-C₄)fluoralkyl, —C(O)—(C₁-C₄)alkylamine, and —C(O)—(C₁-C₄)alkoxy; R_{1b} is phenyl, optionally substituted with 1-4 moieties independently selected from the group consisting of halogen, —CN, -L₁-OH, -L₁-NH₂, -L₁-(C₁-C₄)alkyl, -L₁-(C₃-C₆)cycloalkyl, -L₁-(C₁-C₄)fluoroalkyl, -L₁-(C₁-C₄)alkoxy, -L₁-(C₁-C₄)alkylamine, -L₁-(C₁-C₄)dialkylamine and -L₁-phenyl, wherein L₁ is a bond, —C(O)— and S(O)₂, are provided herein. In some embodiments, z is 0; or z is 1 or 2 and R₁ₐ is H; or z is 1 or 2 and R₁ₐ is (C₁-C₄)alkyl.

Provided herein are compositions, methods of treating, and methods for modulating the activity of epidermal growth factor receptor comprising providing an effective amount of a compound of any of Formula Ia, Formula Iaa, Formula Ib and Formula Ibb wherein R₄ is a moiety having the structure —(CHR₄ₐ)_{y}—R_{4b}, wherein y is a number selected from the group consisting of 0, 1, 2 and 3; R₄ₐ is a moiety selected from the group consisting of H, (C₁-C₄)alkyl, F, (C₁-C₄)fluoroalkyl, (C₁-C₄)alkoxy, —(C₁-C₄)alkylamine, —(C₁-C₄)dialkylamine; and R_{4b} is a moiety selected from the group consisting of —(C₁-C₄)alkyl, an optionally substituted —(C₃-C₆)cycloalkyl, —(C₁-C₄)fluoroalkyl, an optionally substituted phenyl, and an optionally substituted 5-membered or 6-membered unsaturated heterocycle; or R_{4b} is H when y is 1, 2, or 3, are also provided herein. In some embodiments, y is 0 or 1 and R₄ₐ is H; or y is 0 or 1 and R₄ₐ is (C₁-C₄)alkyl. In other embodiments, R₆ is an H; or R₆ is an optionally substituted phenyl; or R₆ is an optionally substituted heteroaryl.

Provided herein are compositions, methods of treating, and methods for modulating the activity of epidermal growth factor receptor comprising providing an effective amount of a compound of any of Formula Ia, Formula Iaa, Formula Ib and Formula Ibb wherein R₁ is a moiety having the structure —(CHR₁ₐ)_{z}—R_{1b}, wherein z is a number selected from the group consisting of 0, 1, 2 and 3; R₁ₐ is a moiety selected from the group consisting of H, (C₁-C₄)alkyl, F, (C₁-C₄)fluoroalkyl, (C₁-C₄)alkoxy, —(C₁-C₄)alkylamine, —(C₁-C₄)dialkylamine, —C(O)OH, —C(O)—NH₂, —C(O)—(C₁-C₄)alkyl, —C(O)—(C₁-C₄)fluoralkyl, —C(O)—(C₁-C₄)alkylamine, and —C(O)—(C₁-C₄)alkoxy; R_{1b} is a moiety selected from the group consisting of —(C₁-C₄)alkyl, an optionally substituted —(C₃-C₆)cycloalkyl, —(C₁-C₄)fluoroalkyl, and an optionally substituted 5-membered or 6-membered unsaturated heterocycle; or R_{1b} is H when z is 1, 2, or 3, are provided herein. In some embodiments, z is 0; or z is 1 and R₁ₐ is H or (C₁-C₄)alkyl.

Provided herein are compositions, methods of treating, and methods for modulating the activity of epidermal growth factor receptor comprising providing an effective amount of a compound of any of Formula Ia, Formula Iaa, Formula Ib and Formula Ibb wherein R₅ is a phenyl, optionally substituted with 1-2 moieties independently selected from the group consisting of halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, —(C₁-C₄)dialkylamine, —C(O)OH, —C(O)—NH₂, —C(O)—(C₁-C₄)alkyl, —C(O)—(C₁-C₄)fluoralkyl, —C(O)—(C₁-C₄)alkylamine, and —C(O)—(C₁-C₄)alkoxy, are provided herein. In some embodiments, R₆ is an H; or R₆ is an optionally substituted phenyl; or R₆ is an optionally substituted heteroaryl. In other embodiments, R₁ is a moiety having the structure —(CHR₁ₐ)_{z}—R_{1b}, wherein z is a number selected from the group consisting of 0, 1, 2 and 3; R₁ₐ is a moiety selected from the group consisting of H, (C₁-C₄)alkyl, F, (C₁-C₄)fluoroalkyl, (C₁-C₄)alkoxy, —C(O)OH, —C(O)—NH₂, —C(O)—(C₁-C₄)alkyl, —C(O)—(C₁-C₄)fluoralkyl, —C(O)—(C₁-C₄)alkylamine, and —C(O)—(C₁-C₄)alkoxy; and R_{1b} is phenyl, optionally substituted with 1-4 moieties independently selected from the group consisting of halogen, —CN, -L₁-OH, -L₁-NH₂, -L₁-(C₁-C₄)alkyl, -L₁-(C₃-C₆)cycloalkyl, -L₁-(C₁-C₄)fluoroalkyl, -L₁-(C₁-C₄)alkoxy, -L₁-(C₁-C₄)alkylamine, -L₁-(C₁-C₄)dialkylamine and -L₁-phenyl, wherein L₁ is a bond, —C(O)— and S(O)₂. In other embodiments, R₁ is a moiety having the structure —(CHR₁ₐ)_{z}—R_{1b}, wherein z is a number selected from the group consisting of 0, 1, 2 and 3; R₁ₐ is a moiety selected from the group consisting of H, (C₁-C₄)alkyl, F, (C₁-C₄)fluoroalkyl, (C₁-C₄)alkoxy, —(C₁-C₄)alkylamine, —(C₁-C₄)dialkylamine, —C(O)OH, —C(O)—NH₂, —C(O)—(C₁-C₄)alkyl, —C(O)—(C₁-C₄)fluoralkyl, —C(O)—(C₁-C₄)alkylamine, and —C(O)—(C₁-C₄)alkoxy; and R_{1b} is a moiety selected from the group consisting of —(C₁-C₄)alkyl, an optionally substituted —(C₃-C₆)cycloalkyl, —(C₁-C₄)fluoroalkyl, and an optionally substituted 5-membered or 6-membered unsaturated heterocycle; or R_{1b} is H when z is 1, 2, or 3. In still other embodiments, z is 0; or z is 1 and R₁ₐ is H or (C₁-C₄)alkyl.

Provided herein are compositions, methods of treating, and methods for modulating the activity of epidermal growth factor receptor comprising providing an effective amount of a compound of any of Formula Ia, Formula Iaa, Formula Ib and Formula Ibb wherein R₄ is a moiety having the structure —(CHR₄ₐ)_{y}—R_{4b}, wherein y is a number selected from the group consisting of 0, 1, 2 and 3; R₄ₐ is a moiety selected from the group consisting of H, (C₁-C₄)alkyl, F, (C₁-C₄)fluoroalkyl, (C₁-C₄)alkoxy, —(C₁-C₄)alkylamine, —(C₁-C₄)dialkylamine; R_{4b} is a moiety selected from the group consisting of —(C₁-C₄)alkyl, an optionally substituted —(C₃-C₆)cycloalkyl, —(C₁-C₄)fluoroalkyl, an optionally substituted phenyl, and an optionally substituted 5-membered or 6-membered unsaturated heterocycle; or R_{4b} is H when y is 1, 2, or 3; R₅ is H or phenyl, optionally substituted with 1-2 moieties independently selected from the group consisting of halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, —(C₁-C₄)dialkylamine, —C(O)OH, —C(O)—NH₂, —C(O)—(C₁-C₄)alkyl, —C(O)—(C₁-C₄)fluoralkyl, —C(O)—(C₁-C₄)alkylamine, and —C(O)—(C₁-C₄)alkoxy; and R₆ is a moiety selected from the group consisting of H, heteroaryl, and phenyl, wherein the phenyl and the heteroaryl are optionally substituted with 1-2 moieties independently selected from the group consisting of halogen, —(C₁-C₄)alkyl, —(C₁-C₄)fluoroalkyl, —(C₁-C₄)alkoxy, —(C₁-C₄)alkylamine, and —(C₁-C₄)dialkylamine; or R₅ and R₆ together form a 6-membered carbocyclic aromatic ring structure, optionally substituted with 1-2 moieties independently selected from the group consisting of halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, and —(C₁-C₄)dialkylamine, are provided herein. In some embodiments, R₅ is the optionally substituted phenyl. In other embodiments, R₆ is an H; or R₆ is an optionally substituted phenyl; or R₆ is an optionally substituted heteroaryl. In other embodiments, R₁ is a moiety having the structure —(CHR₁ₐ)_{z}—R_{1b}, wherein z is a number selected from the group consisting of 0, 1, 2 and 3; R₁ₐ is a moiety selected from the group consisting of H, (C₁-C₄)alkyl, F, (C₁-C₄)fluoroalkyl, (C₁-C₄)alkoxy, —C(O)OH, —C(O)—NH₂, —C(O)—(C₁-C₄)alkyl, —C(O)—(C₁-C₄)fluoralkyl, —C(O)—(C₁-C₄)alkylamine, and —C(O)—(C₁-C₄)alkoxy; and R_{1b} is phenyl, optionally substituted with 1-4 moieties independently selected from the group consisting of halogen, —CN, -L₁-OH, -L₁-NH₂, -L₁-(C₁-C₄)alkyl, -L₁-(C₃-C₆)cycloalkyl, -L₁-(C₁-C₄)fluoroalkyl, -L₁-(C₁-C₄)alkoxy, -L₁-(C₁-C₄)alkylamine, -L₁-(C₁-C₄)dialkylamine and -L₁-phenyl, wherein L₁ is a bond, —C(O)— and S(O)₂. In still other embodiments, z is 0; or z is 1 or 2 and R₁ₐ is H; or z is 1 or 2 and R₁ₐ is (C₁-C₄)alkyl. In yet other embodiments, R₁ is a moiety having the structure —(CHR₁ₐ)_{z}—R_{1b}, wherein z is a number selected from the group consisting of 0, 1, 2 and 3; R₁ₐ is a moiety selected from the group consisting of H, (C₁-C₄)alkyl, F, (C₁-C₄)fluoroalkyl, (C₁-C₄)alkoxy, —(C₁-C₄)alkylamine, —(C₁-C₄)dialkylamine, —C(O)OH, —C(O)—NH₂, —C(O)—(C₁-C₄)alkyl, —C(O)—(C₁-C₄)fluoralkyl, —C(O)—(C₁-C₄)alkyl amine, and —C(O)—(C₁-C₄)alkoxy; and R_{1b} is a moiety selected from the group consisting of —(C₁-C₄)alkyl, an optionally substituted —(C₃-C₆)cycloalkyl, —(C₁-C₄)fluoroalkyl, and an optionally substituted 5-membered or 6-membered unsaturated heterocycle; or R_{1b} is H when z is 1, 2, or 3.

Provided herein are compositions, methods of treating, and methods for modulating the activity of epidermal growth factor receptor comprising providing an effective amount of a compound of any of Formula Ia, Formula Iaa, Formula Ib and Formula Ibb wherein R₄, R₅ and R₆ are each H. In some embodiments, R₁ is a moiety having the structure —(CHR₁ₐ)_{z}—R_{1b}, wherein z is a number selected from the group consisting of 0, 1, 2 and 3; R₁ₐ is a moiety selected from the group consisting of H, (C₁-C₄)alkyl, F, (C₁-C₄)fluoroalkyl, (C₁-C₄)alkoxy, —C(O)OH, —C(O)—NH₂, —C(O)—(C₁-C₄)alkyl, —C(O)—(C₁-C₄)fluoralkyl, —C(O)—(C₁-C₄)alkylamine, and —C(O)—(C₁-C₄)alkoxy; and R_{1b} is phenyl, optionally substituted with 1-4 moieties independently selected from the group consisting of halogen, —CN, -L₁-OH, -L₁-NH₂, -L₁-(C₁-C₄)alkyl, -L₁-(C₃-C₆)cycloalkyl, -L₁-(C₁-C₄)fluoroalkyl, -L₁-(C₁-C₄)alkoxy, -L₁-(C₁-C₄)alkylamine, -L₁-C₁-C₄)dialkylamine and -L₁-phenyl, wherein L₁ is a bond, —C(O)— and S(O)₂. In still other embodiments, R_{1b} is phenyl, optionally substituted with 1 moiety selected from the group consisting of halogen, —CN, -L₁-OH, -L₁-NH₂, -L₁-(C₁-C₄)alkyl, -L₁-(C₃-C₆)cycloalkyl, -L₁-(C₁-C₄)fluoroalkyl, -L₁-(C₁-C₄)alkoxy, -L₁-(C₁-C₄)alkylamine, -L₁-C₁-C₄)dialkylamine and -L₁-phenyl, wherein L₁ is a bond, —C(O)— and S(O)₂.

Provided herein are compositions, methods of treating, and methods for modulating the activity of epidermal growth factor receptor comprising providing an effective amount of a compound of any of Formula Ia, Formula Iaa, Formula Ib and Formula Ibb wherein R₄ is —(C₁-C₄)alkyl; R₅ is phenyl, optionally substituted with 1-2 moieties independently selected from the group consisting of halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, —(C₁-C₄)dialkylamine, —C(O)OH, —C(O)—NH₂, —C(O)—(C₁-C₄)alkyl, —C(O)—(C₁-C₄)fluoralkyl, —C(O)—(C₁-C₄)alkylamine, and —C(O)—(C₁-C₄)alkoxy; and R₆ is a moiety selected from the group consisting of H and phenyl, optionally substituted with 1-2 moieties independently selected from the group consisting of halogen, —(C₁-C₄)alkyl, —(C₁-C₄)fluoroalkyl, —(C₁-C₄)alkoxy, —(C₁-C₄)alkylamine, and —(C₁-C₄)dialkylamine, are provided herein. In some embodiments, R₆ is H. In other embodiments, R₅ is phenyl, optionally substituted with 1-2 moieties independently selected from the group consisting of —OH, and —(C₁-C₄)alkoxy; or R₅ is phenyl, optionally substituted with 1-2 moieties independently selected from the group consisting of halogen, —CN, —NH₂, —(C₁-C₄)alkyl, —(C₃-C₆)cycloalkyl, —(C₁-C₄)fluoroalkyl, —(C₁-C₄)alkylamine, —(C₁-C₄)dialkylamine, —C(O)OH, —C(O)—NH₂, —C(O)—(C₁-C₄)alkyl, —C(O)—(C₁-C₄)fluoralkyl, —C(O)—(C₁-C₄)alkylamine, and —C(O)—(C₁-C₄)alkoxy.

Provided herein are compositions, methods of treating, and methods for modulating the activity of epidermal growth factor receptor comprising providing an effective amount of a compound of any of Formula Ia, Formula Iaa; Formula Ib and Formula Ibb wherein R₄ is an optionally substituted —(C₃-C₆)cycloalkyl; R₅ is H or phenyl, optionally substituted with 1-2 moieties independently selected from the group consisting of halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, —(C₁-C₄)dialkylamine, —C(O)OH, —C(O)—NH₂, —C(O)—(C₁-C₄)alkyl, —C(O)—(C₁-C₄)fluoralkyl, —C(O)—(C₁-C₄)alkylamine, and —C(O)—(C₁-C₄)alkoxy; and R₆ is a moiety selected from the group consisting of H and phenyl, optionally substituted with 1-2 moieties independently selected from the group consisting of halogen, —(C₁-C₄)alkyl, —(C₁-C₄)fluoroalkyl, —(C₁-C₄)alkoxy, —(C₁-C₄)alkylamine, and —(C₁-C₄)dialkylamine, are provided herein. In some embodiments, R₆ is H. In other embodiments, R₅ is phenyl, optionally substituted with 1-2 moieties independently selected from the group consisting of —OH, and —(C₁-C₄)alkoxy. In still other embodiments, R₅ is phenyl, optionally substituted with 1-2 moieties independently selected from the group consisting of halogen, —CN, —NH₂, —(C₁-C₄)alkyl, —(C₃-C₆)cycloalkyl, —(C₁-C₄)fluoroalkyl, —(C₁-C₄)alkylamine, —(C₁-C₄)dialkylamine, —C(O)OH, —C(O)—NH₂, —C(O)—(C₁-C₄)alkyl, —C(O)—(C₁-C₄)fluoralkyl, —C(O)—(C₁-C₄)alkylamine, and —C(O)—(C₁-C₄)alkoxy.

Provided herein are compositions, methods of treating, and methods for modulating the activity of epidermal growth factor receptor comprising providing an effective amount of a compound of any of Formula Ia, Formula Iaa, Formula Ib and Formula Ibb wherein R₄ is a CH₂ group substituted by an optionally substituted phenyl; R₅ is H or phenyl, optionally substituted with 1-2 moieties independently selected from the group consisting of halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, —(C₁-C₄)dialkylamine, —C(O)OH, —C(O)—NH₂, —C(O)—(C₁-C₄)alkyl, —C(O)—(C₁-C₄)fluoralkyl, —C(O)—(C₁-C₄)alkylamine, and —C(O)—(C₁-C₄)alkoxy; and R₆ is a moiety selected from the group consisting of H and phenyl, optionally substituted with 1-2 moieties independently selected from the group consisting of halogen, —(C₁-C₄)alkyl, —(C₁-C₄)fluoroalkyl, —(C₁-C₄)alkoxy, —(C₁-C₄)alkylamine, and —(C₁-C₄)dialkylamine, are provided herein. In some embodiments, R₆ is H. In other embodiments, R₅ is phenyl, optionally substituted with 1-2 moieties independently selected from the group consisting of —OH, and —(C₁-C₄)alkoxy. In still other embodiments, R₅ is phenyl, optionally substituted with 1-2 moieties independently selected from the group consisting of halogen, —CN, —NH₂, —(C₁-C₄)alkyl, —(C₃-C₆)cycloalkyl, —(C₁-C₄)fluoroalkyl, —(C₁-C₄)alkylamine, —(C₁-C₄)dialkylamine, —C(O)OH, —C(O)—NH₂, —C(O)—(C₁-C₄)alkyl, —C(O)—(C₁-C₄)fluoralkyl, —C(O)—(C₁-C₄)alkylamine, and —C(O)—(C₁-C₄)alkoxy. In yet other embodiments, R₁ is a moiety having the structure —(CHR₁ₐ)_{z}—R_{1b}, wherein z is a number selected from the group consisting of 0, 1, 2 and 3; R₁ₐ is a moiety selected from the group consisting of H, (C₁-C₄)alkyl, F, (C₁-C₄)fluoroalkyl, (C₁-C₄)alkoxy, —C(O)OH, —C(O)—NH₂, —C(O)—(C₁-C₄)alkyl, —C(O)—(C₁-C₄)fluoralkyl, —C(O)—(C₁-C₄)alkylamine, and —C(O)—(C₁-C₄)alkoxy; R_{1b} is phenyl, optionally substituted with 1-4 moieties independently selected from the group consisting of halogen, —CN, -L₁-OH, -L₁-NH₂, -L₁-(C₁-C₄)alkyl, -L₁-(C₃-C₆)cycloalkyl, -L₁-(C₁-C₄)fluoroalkyl, -L₁-(C₁-C₄)alkoxy, -L₁-(C₁-C₄)alkylamine, -L₁-(C₁-C₄)dialkylamine and -L₁-phenyl, wherein L₁ is a bond, —C(O)— and S(O)₂. And still in other embodiments, R₁ is a moiety having the structure —(CHR₁ₐ)_{z}—R_{1b}, wherein z is a number selected from the group consisting of 0, 1, 2 and 3; R₁ₐ is a moiety selected from the group consisting of H, (C₁-C₄)alkyl, F, (C₁-C₄)fluoroalkyl, (C₁-C₄)alkoxy, —(C₁-C₄)alkylamine, —(C₁-C₄)dialkylamine, —C(O)OH, —C(O)—NH₂, —C(O)—(C₁-C₄)alkyl, —C(O)—(C₁-C₄)fluoralkyl, —C(O)—(C₁-C₄)alkylamine, and —C(O)—(C₁-C₄)alkoxy; and R_{1b} is a moiety selected from the group consisting of —(C₁-C₄)alkyl, an optionally substituted —(C₃-C₆)cycloalkyl, —(C₁-C₄)fluoroalkyl, and an optionally substituted 5-membered or 6-membered unsaturated heterocycle; or R_{1b} is H when z is 1, 2, or 3.

Provided herein are compositions, methods of treating, and methods for modulating the activity of epidermal growth factor receptor comprising providing an effective amount of a compound of any of Formula Ia, Formula Iaa, Formula Ib and Formula Ibb wherein R₃ is —(CHR₃ₐ)ₓ—R_{3b}, wherein x is 0, 1, 2, or 3; and R₃ₐ is selected from the group consisting of H, (C₁-C₄)alkyl, F, (C₁-C₄)fluoroalkyl, (C₁-C₄)alkoxy, —(C₁-C₄)alkylamine, and —(C₁-C₄)dialkylamine; and R_{3b} is H or a phenyl, optionally substituted with 1-2 substituents independently selected from the group consisting of halogen, —(C₁-C₄)alkyl, —(C₁-C₄)fluoroalkyl, —(C₁-C₄)alkoxy, —(C₁-C₄)alkylamine, and —(C₁-C₄)dialkylamine, are also provided herein.

Provided herein are compositions, methods of treating, and methods for modulating the activity of epidermal growth factor receptor comprising providing an effective amount of a compound of any of Formula Ia, Formula Iaa, Formula Ib and Formula Ibb wherein R₃ is —NH—(CHR₃ₐ)ₓ—R_{3b}, wherein x is 0, 1, 2, or 3; and R₃ₐ is selected from the group consisting of H, (C₁-C₄)alkyl, F, (C₁-C₄)fluoroalkyl, (C₁-C₄)alkoxy, —(C₁-C₄)alkylamine, and —(C₁-C₄)dialkylamine; and R_{3b} is H or a phenyl, optionally substituted with 1-2 substituents independently selected from the group consisting of halogen, —(C₁-C₄)alkyl, —(C₁-C₄)fluoroalkyl, —(C₁-C₄)alkoxy, —(C₁-C₄)alkylamine, and —(C₁-C₄)dialkylamine, are also provided herein.

Provided herein are compositions, methods of treating, and methods for modulating the activity of epidermal growth factor receptor comprising providing an effective amount of a compound of any of Formula Ia, Formula Iaa, Formula Ib and Formula Ibb wherein R₃ is —O—(CHR₃ₐ)ₓ—R_{3b}, wherein x is 0, 1, 2, or 3; and R₃ₐ is selected from the group consisting of H, (C₁-C₄)alkyl, F, (C₁-C₄)fluoroalkyl, (C₁-C₄)alkoxy, —(C₁-C₄)alkylamine, and —(C₁-C₄)dialkylamine; and R_{3b} is H or a phenyl, optionally substituted with 1-2 substituents independently selected from the group consisting of halogen, —(C₁-C₄)alkyl, —(C₁-C₄)fluoroalkyl, —(C₁-C₄)alkoxy, —(C₁-C₄)alkylamine, and —(C₁-C₄)dialkylamine, are also provided herein.

Provided herein are compositions, methods of treating, and methods for modulating the activity of epidermal growth factor receptor comprising providing an effective amount of a compound of any of Formula Ia, Formula Iaa, Formula Ib and Formula Ibb wherein R₃ is —S—(CHR₃ₐ)ₓ—R_{3b}, wherein x is 0, 1, 2, or 3; and R₃ₐ is selected from the group consisting of H, (C₁-C₄)alkyl, F, (C₁-C₄)fluoroalkyl, (C₁-C₄)alkoxy, —(C₁-C₄)alkylamine, and —(C₁-C₄)dialkylamine; and R_{3b} is H or a phenyl, optionally substituted with 1-2 substituents independently selected from the group consisting of halogen, —(C₁-C₄)alkyl, —(C₁-C₄)fluoroalkyl, —(C₁-C₄)alkoxy, —(C₁-C₄)alkylamine, and —(C₁-C₄)dialkylamine, are provided herein.

Provided herein are compositions, methods of treating, and methods for modulating the activity of epidermal growth factor receptor comprising providing an effective amount of a compound of any of Formula Ia, IIaa, IIb or IIbb: 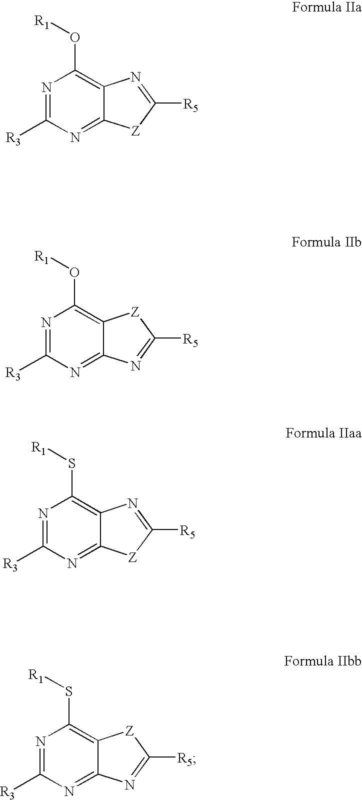
wherein Z is selected from the group consisting of O, S, and NR_{4;}
(a) R₁ is selected from one of the following options:
   a. R₁ is a moiety having the structure —(CHR₁ₐ)_{z}—R_{1b},
      i. wherein z is a number selected from the group consisting of 0, 1, 2 and 3;
      ii. R₁ₐ is a moiety selected from the group consisting of H, (C₁-C₄)alkyl, F, (C₁-C₄)fluoroalkyl, (C₁-C₄)alkoxy, —C(O)OH, —C(O)—NH₂, —C(O)—(C₁-C₄)alkyl, —C(O)—(C₁-C₄)fluoralkyl, —C(O)—(C₁-C₄)alkylamine, and —C(O)—(C₁-C₄)alkoxy; and
      iii. R_{1b} is phenyl, optionally substituted with 1-4 moieties independently selected from the group consisting of halogen, —CN, -L₁-OH, -L₁-NH₂, -L₁-(C₁-C₄)alkyl, -L₁-(C₃-C₆)cycloalkyl, -L₁-(C₁-C₄)fluoroalkyl, -L₁-(C₁-C₄)alkoxy, -L₁-(C₁-C₄)alkylamine, -L₁-(C₁-C₄)dialkylamine and -L₁-phenyl, wherein L₁ is a bond, —C(O)— and S(O)₂; or
   b. R₁ is a moiety having the structure —(CHR₁ₐ)_{z}—R_{1b},
      i. wherein z is a number selected from the group consisting of 0, 1, 2 and 3;
      ii. R₁ₐ is a moiety selected from the group consisting of H, (C₁-C₄)alkyl, F, (C₁-C₄)fluoroalkyl, (C₁-C₄)alkoxy, —(C₁-C₄)alkylamine, —(C₁-C₄)dialkylamine, —C(O)OH, —C(O)—NH₂, —C(O)—(C₁-C₄)alkyl, —C(O)—(C₁-C₄)fluoralkyl, —C(O)—(C₁-C₄)alkylamine, and —C(O)—(C₁-C₄)alkoxy; and
      iii. R_{1b} is a moiety selected from the group consisting of —(C₁-C₄)alkyl, an optionally substituted —(C₃-C₆)cycloalkyl, —(C₁-C₄)fluoroalkyl, and an optionally substituted 5-membered or 6-membered unsaturated heterocycle; or R_{1b} is H when z is 1, 2, or 3; and
(b) R₃ is L₃-(CHR₃ₐ)ₓ—R_{3b}, wherein x is 0, 1, 2, or 3; L₃ is a bond, NH, O or S; R₃ₐ is selected from the group consisting of H, (C₁-C₄)alkyl, F, (C₁-C₄)fluoroalkyl, (C₁-C₄)alkoxy, —(C₁-C₄)alkylamine, and —(C₁-C₄)dialkylamine; and R_{3b} is H or a phenyl, optionally substituted with 1-2 substituents independently selected from the group consisting of halogen, —(C₁-C₄)alkyl, —(C₁-C₄)fluoroalkyl, —(C₁-C₄)alkoxy, —(C₁-C₄)alkylamine, and —(C₁-C₄)dialkylamine;
(c) R₄ is H or a moiety having the structure —(CHR₄ₐ)_{y}—R_{4b},
   i. wherein y is a number selected from the group consisting of 0, 1, 2 and 3;
   ii. R₄ₐ is a moiety selected from the group consisting of H, (C₁-C₄)alkyl, F, (C₁-C₄)fluoroalkyl, (C₁-C₄)alkoxy, —(C₁-C₄)alkylamine, —(C₁-C₄)dialkylamine; and
   iii. R_{4b} is a moiety selected from the group consisting of —(C₁-C₄)alkyl, an optionally substituted —(C₃-C₆)cycloalkyl, —(C₁-C₄)fluoroalkyl, an optionally substituted phenyl, and an optionally substituted 5-membered or 6-membered unsaturated heterocycle; or R_{4b} is H when y is 1, 2, or 3; and
(d) R₅ is H or phenyl, optionally substituted with 1-2 moieties independently selected from the group consisting of halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, —(C₁-C₄)dialkylamine, —C(O)OH, —C(O)—NH₂, —C(O)—(C₁-C₄)alkyl, —C(O)—(C₁-C₄)fluoralkyl, —C(O)—(C₁-C₄)alkylamine, and —C(O)—(C₁-C₄)alkoxy; or
   R₁ and R₄ together, when the compound has the structure of Formula IIb or Formula IIbb form a 5- or 6-membered heterocyclic aromatic ring structure, optionally substituted with 1-2 moieties independently selected from the group consisting of halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, and —(C₁-C₄)dialkylamine; or
   R₄ and R₅ together form a 5- or 6-membered heterocyclic aromatic ring structure, optionally substituted with 1-2 moieties independently selected from the group consisting of halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, and —(C₁-C₄)dialkylamine;
   or a pharmaceutically acceptable salt, pharmaceutically acceptable N-oxide, pharmaceutically active metabolite, pharmaceutically acceptable prodrug, or pharmaceutically acceptable solvate thereof.

Provided herein are compositions, methods of treating, and methods for modulating the activity of epidermal growth factor receptor comprising providing an effective amount of a compound of any of Formula Ia, IIaa, IIb or IIbb wherein R₄ is a moiety having the structure —(CHR₄ₐ)_{y}—R_{4b}, wherein y is a number selected from the group consisting of 0, 1, 2 and 3; R₄ₐ is a moiety selected from the group consisting of H, (C₁-C₄)alkyl, F, (C₁-C₄)fluoroalkyl, (C₁-C₄)alkoxy, —(C₁-C₄)alkylamine, —(C₁-C₄)dialkylamine; and R_{4b} is a moiety selected from the group consisting of —(C₁-C₄)alkyl, an optionally substituted —(C₃-C₆)cycloalkyl, —(C₁-C₄)fluoroalkyl, an optionally substituted phenyl, and an optionally substituted 5-membered or 6-membered unsaturated heterocycle; or R_{4b} is H when y is 1, 2, or 3, are provided herein. In some embodiments, R₁ is a moiety having the structure —(CHR₁ₐ)_{z}—R_{1b}, wherein z is a number selected from the group consisting of 0, 1, 2 and 3; R₁ₐ is a moiety selected from the group consisting of H, (C₁-C₄)alkyl, F, (C₁-C₄)fluoroalkyl, (C₁-C₄)alkoxy, —C(O)OH, —C(O)—NH₂, —C(O)—(C₁-C₄)alkyl, —C(O)—(C₁-C₄)fluoralkyl, —C(O)—(C₁-C₄)alkylamine, and —C(O)—(C₁-C₄)alkoxy; and R_{1b} is phenyl, optionally substituted with 1-4 moieties independently selected from the group consisting of halogen, —CN, -L₁-OH, -L₁-NH₂, -L₁-(C₁-C₄)alkyl, -L₁-(C₃-C₆)cycloalkyl, -L₁-(C₁-C₄)fluoroalkyl, -L₁-(C₁-C₄)alkoxy, -L₁-(C₁-C₄)alkylamine, -L₁-C₁-C₄)dialkylamine and -L₁-phenyl, wherein L₁ is a bond, —C(O)— and S(O)₂. In other embodiments, z is 0; or z is 1 and R₁ₐ is a moiety selected from the group consisting of H and (C₁-C₄)alkyl.

Provided herein are compositions, methods of treating, and methods for modulating the activity of epidermal growth factor receptor comprising providing an effective amount of a compound of any of Formula Ia, IIaa, IIb or IIbb wherein R₁ is a moiety having the structure —(CHR₁ₐ)_{z}—R_{1b}, wherein z is a number selected from the group consisting of 0, 1, 2 and 3; R₁ₐ is a moiety selected from the group consisting of H, (C₁-C₄)alkyl, F, (C₁-C₄)fluoroalkyl, (C₁-C₄)alkoxy, —(C₁-C₄)alkylamine, —(C₁-C₄)dialkylamine, —C(O)OH, —C(O)—NH₂, —C(O)—(C₁-C₄)alkyl, —C(O)—(C₁-C₄)fluoralkyl, —C(O)—(C₁-C₄)alkylamine, and —C(O)—(C₁-C₄)alkoxy; and R_{1b} is a moiety selected from the group consisting of —(C₁-C₄)alkyl, an optionally substituted —(C₃-C₆)cycloalkyl, —(C₁-C₄)fluoroalkyl, and an optionally substituted 5-membered or 6-membered unsaturated heterocycle; or R_{1b} is H when z is 1, 2, or 3; are provided herein. In some embodiments, z is 0; or z is 1 and R₁ₐ is a moiety selected from the group consisting of H and (C₁-C₄)alkyl. In other embodiments, R₁ is a moiety having the structure —(CHR₁ₐ)_{z}—R_{1b}, wherein z is a number selected from the group consisting of 0, 1, 2 and 3; R₁ₐ is a moiety selected from the group consisting of H, (C₁-C₄)alkyl, F, (C₁-C₄)fluoroalkyl, (C₁-C₄)alkoxy, —C(O)OH, —C(O)—NH₂, —C(O)—(C₁-C₄)alkyl, —C(O)—(C₁-C₄)fluoralkyl, —C(O)—(C₁-C₄)alkylamine, and —C(O)—(C₁-C₄)alkoxy; and R_{1b} is phenyl, optionally substituted with 1-4 moieties independently selected from the group consisting of halogen, —CN, -L₁-OH, -L₁-NH₂, -L₁-(C₁-C₄)alkyl, -L₁-(C₃-C₆)cycloalkyl, -L₁-(C₁-C₄)fluoroalkyl, -L₁-(C₁-C₄)alkoxy, -L₁-(C₁-C₄)alkylamine, -L₁-(C₁-C₄)dialkylamine and -L₁-phenyl, wherein L₁ is a bond, —C(O)— and S(O)₂. In still other embodiments, R₁ is a moiety having the structure —(CHR₁ₐ)_{z}—R_{1b}, wherein z is a number selected from the group consisting of 0, 1, 2 and 3; R₁ₐ is a moiety selected from the group consisting of H, (C₁-C₄)alkyl, F, (C₁-C₄)fluoroalkyl, (C₁-C₄)alkoxy, —(C₁-C₄)alkylamine, —(C₁-C₄)dialkylamine, —C(O)OH, —C(O)—NH₂, —C(O)—(C₁-C₄)alkyl, —C(O)—(C₁-C₄)fluoralkyl, —C(O)—(C₁-C₄)alkylamine, and —C(O)—(C₁-C₄)alkoxy; and R_{1b} is a moiety selected from the group consisting of —(C₁-C₄)alkyl, an optionally substituted —(C₃-C₆)cycloalkyl, —(C₁-C₄)fluoroalkyl, and an optionally substituted 5-membered or 6-membered unsaturated heterocycle; or R_{1b} is H when z is 1, 2, or 3.

Provided herein are compositions, methods of treating, and methods for modulating the activity of epidermal growth factor receptor comprising providing an effective amount of a compound of any of Formula IIa, IIaa, IIb or IIbb wherein R₃ is —(CHR₃ₐ)ₓ—R_{3b}, wherein x is 0, 1, 2, or 3; and R₃ₐ is selected from the group consisting of H, (C₁-C₄)alkyl, F, (C₁-C₄)fluoroalkyl, (C₁-C₄)alkoxy, —(C₁-C₄)alkylamine, and —(C₁-C₄)dialkylamine; and R_{3b} is H or a phenyl, optionally substituted with 1-2 substituents independently selected from the group consisting of halogen, —(C₁-C₄)alkyl, —(C₁-C₄)fluoroalkyl, —(C₁-C₄)alkoxy, —(C₁-C₄)alkylamine, and —(C₁-C₄)dialkylamine, are provided herein. In some embodiments, R₃ is —NH—(CHR₃ₐ)ₓ—R_{3b}, wherein x is 0, 1, 2, or 3; and R₃ₐ is selected from the group consisting of H, (C₁-C₄)alkyl, F, (C₁-C₄)fluoroalkyl, (C₁-C₄)alkoxy, —(C₁-C₄)alkylamine, and —(C₁-C₄)dialkylamine; and R_{3b} is H or a phenyl, optionally substituted with 1-2 substituents independently selected from the group consisting of halogen, —(C₁-C₄)alkyl, —(C₁-C₄)fluoroalkyl, —(C₁-C₄)alkoxy, —(C₁-C₄)alkylamine, and —(C₁-C₄)dialkylamine. In other embodiments, R₃ is —O—(CHR₃ₐ)ₓ—R_{3b}, wherein x is 0, 1, 2, or 3; and R₃ₐ is selected from the group consisting of H, (C₁-C₄)alkyl, F, (C₁-C₄)fluoroalkyl, (C₁-C₄)alkoxy, —(C₁-C₄)alkylamine, and —(C₁-C₄)dialkylamine; and R_{3b} is H or a phenyl, optionally substituted with 1-2 substituents independently selected from the group consisting of halogen, —(C₁-C₄)alkyl, —(C₁-C₄)fluoroalkyl, —(C₁-C₄)alkoxy, —(C₁-C₄)alkylamine, and —(C₁-C₄)dialkylamine. In still other embodiments, R₃ is —S—(CHR₃ₐ)ₓ—R_{3b}, wherein x is 0, 1, 2, or 3; and R₃ₐ is selected from the group consisting of H, (C₁-C₄)alkyl, F, (C₁-C₄)fluoroalkyl, (C₁-C₄)alkoxy, —(C₁-C₄)alkylamine, and —(C₁-C₄)dialkylamine; and R_{3b} is H or a phenyl, optionally substituted with 1-2 substituents independently selected from the group consisting of halogen, —(C₁-C₄)alkyl, —(C₁-C₄)fluoroalkyl, —(C₁-C₄)alkoxy, —(C₁-C₄)alkylamine, and —(C₁-C₄)dialkylamine.

Provided herein are compositions, methods of treating, and methods for modulating the activity of epidermal growth factor receptor comprising providing an effective amount of a compound of any of Formula IIIa, IIIaa, IIIb or IIIbb: 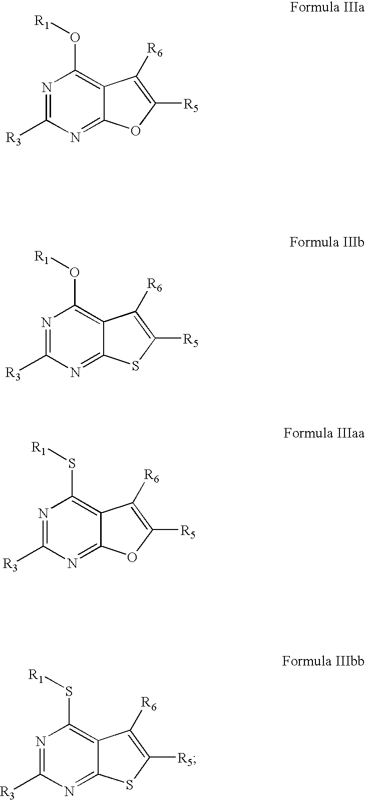
wherein
(a) R₁ is selected from one of the following options:
   a. R₁ is a moiety having the structure —(CHR₁ₐ)_{z}—R_{1b},
      i. wherein z is a number selected from the group consisting of 0, 1, 2 and 3;
      ii. R₁ₐ is a moiety selected from the group consisting of H, (C₁-C₄)alkyl, F, (C₁-C₄)fluoroalkyl, (C₁-C₄)alkoxy, —C(O)OH, —C(O)—NH₂, —C(O)—(C₁-C₄)alkyl, —C(O)—(C₁-C₄)fluoralkyl, —C(O)—(C₁-C₄)alkylamine, and —C(O)—(C₁-C₄)alkoxy; and
      iii. R_{1b} is phenyl, optionally substituted with 1-4 moieties independently selected from the group consisting of halogen, —CN, -L₁-OH, -L₁-NH₂, -L₁-(C₁-C₄)alkyl, -L₁-(C₃-C₆)cycloalkyl, -L₁-(C₁-C₄)fluoroalkyl, -L₁-(C₁-C₄)alkoxy, -L₁-(C₁-C₄)alkylamine, -L₁-(C₁-C₄)dialkylamine and -L₁-phenyl, wherein L₁ is a bond, —C(O)— and S(O)₂; or
   a. R₁ is a moiety having the structure —(CHR₁ₐ)_{z}—R_{1b},
      i. wherein z is a number selected from the group consisting of 0, 1, 2 and 3;
      ii. R₁ₐ is a moiety selected from the group consisting of H, (C₁-C₄)alkyl, F, (C₁-C₄)fluoroalkyl, (C₁-C₄)alkoxy, —(C₁-C₄)alkylamine, —(C₁-C₄)dialkylamine, —C(O)OH, —C(O)—NH₂, —C(O)—(C₁-C₄)alkyl, —C(O)—(C₁-C₄)fluoralkyl, —C(O)—(C₁-C₄)alkylamine, and —C(O)—(C₁-C₄)alkoxy; and
      iii. R_{1b} is a moiety selected from the group consisting of —(C₁-C₄)alkyl, an optionally substituted —(C₃-C₆)cycloalkyl, —(C₁-C₄)fluoroalkyl, and an optionally substituted 5-membered or 6-membered unsaturated heterocycle; or R_{1b} is H when z is 1, 2, or 3; and
(b) R₃ is L₃-(CHR₃ₐ)ₓ—R_{3b}, wherein x is 0, 1, 2, or 3; L₃ is a bond, NH, O or S; R₃ₐ is selected from the group consisting of H, (C₁-C₄)alkyl, F, (C₁-C₄)fluoroalkyl, (C₁-C₄)alkoxy, —(C₁-C₄)alkylamine, and —(C₁-C₄)dialkylamine; and R_{3b} is H or a phenyl, optionally substituted with 1-2 substituents independently selected from the group consisting of halogen, —(C₁-C₄)alkyl, —(C₁-C₄)fluoroalkyl, —(C₁-C₄)alkoxy, —(C₁-C₄)alkylamine, and —(C₁-C₄)dialkylamine;
(c) R₅ is H or phenyl, optionally substituted with 1-2 moieties independently selected from the group consisting of halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, —(C₁-C₄)dialkylamine, —C(O)OH, —C(O)—NH₂, —C(O)—(C₁-C₄)alkyl, —C(O)—(C₁-C₄)fluoralkyl, —C(O)—(C₁-C₄)alkylamine, and —C(O)—(C₁-C₄)alkoxy; and
   R₆ is a moiety selected from the group consisting of H and a phenyl or heteroaryl, wherein the phenyl and the heteroaryl are optionally substituted with 1-2 moieties independently selected from the group consisting of halogen, —(C₁-C₄)alkyl, —(C₁-C₄)fluoroalkyl, —(C₁-C₄)alkoxy, —(C₁-C₄)alkylamine, and —(C₁-C₄)dialkylamine; or
   R₁ and R₆ together, when the compound has the structure of Formula IIIb or IIbb form a 5- or 6-membered heterocyclic aromatic ring structure, optionally substituted with 1-2 moieties independently selected from the group consisting of halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, and —(C₁-C₄)dialkylamine; or
   R₅ and R₆ together form a 5 or 6-membered carbocyclic or heterocyclic ring structure, optionally substituted with 1-2 moieties independently selected from the group consisting of halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, and —(C₁-C₄)dialkylamine, are provided herein;
   or a pharmaceutically acceptable salt, pharmaceutically acceptable N-oxide, pharmaceutically active metabolite, pharmaceutically acceptable prodrug, or pharmaceutically acceptable solvate thereof.

Provided herein are compositions, methods of treating, and methods for modulating the activity of epidermal growth factor receptor comprising providing an effective amount of a compound of any of Formula IIIa, IIIaa, IIIb or IIIbb wherein R₅ is a phenyl, optionally substituted with 1-2 moieties independently selected from the group consisting of halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, —(C₁-C₄)dialkylamine, —C(O)OH, —C(O)—NH₂, —C(O)—(C₁-C₄)alkyl, —C(O)—(C₁-C₄)fluoralkyl, —C(O)—(C₁-C₄)alkylamine, and —C(O)—(C₁-C₄)alkoxy, are provided herein. In some embodiments, the 1-2 optional moieties are independently selected from the group consisting of halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, and —(C₁-C₄)dialkylamine. In other embodiments, R₆ is H. In still other embodiments, R₆ is the optionally substituted heteroaryl group. In still other embodiments, the phenyl group is substituted with 1-2 moieties independently selected from the group consisting of halogen, —(C₁-C₄)alkyl, —(C₁-C₄)fluoroalkyl, —(C₁-C₄)alkoxy, —(C₁-C₄)alkylamine, and —(C₁-C₄)dialkylamine. In yet other embodiments, R₅ and R₆ together form a 6-membered carbocyclic aromatic ring structure, optionally substituted with 1-2 moieties independently selected from the group consisting of halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, and —(C₁-C₄)dialkylamine.

Provided herein are compositions, methods of treating, and methods for modulating the activity of epidermal growth factor receptor comprising providing an effective amount of a compound of any of Formula IIIa, IIIaa, IIIb or IIIbb wherein R₁ is a moiety having the structure —(CHR₁ₐ)_{z}—R_{1b}, wherein z is a number selected from the group consisting of 0, 1, 2 and 3; R₁ₐ is a moiety selected from the group consisting of H, (C₁-C₄)alkyl, F, (C₁-C₄)fluoroalkyl, (C₁-C₄)alkoxy, —C(O)OH, —C(O)—NH₂, —C(O)—(C₁-C₄)alkyl, —C(O)—(C₁-C₄)fluoralkyl, —C(O)—(C₁-C₄)alkylamine, and —C(O)—(C₁-C₄)alkoxy; and R_{1b} is phenyl, optionally substituted with 1-4 moieties independently selected from the group consisting of halogen, —CN, -L₁-OH, -L₁-NH₂, -L₁-(C₁-C₄)alkyl, -L₁-(C₃-C₆)cycloalkyl, -L₁-(C₁-C₄)fluoroalkyl, -L₁-(C₁-C₄)alkoxy, -L₁-(C₁-C₄)alkylamine, -L₁-(C₁-C₄)dialkylamine and -L₁-phenyl, wherein L₁ is a bond, —C(O)— and S(O)₂, are provided herein.

Provided herein are compositions, methods of treating, and methods for modulating the activity of epidermal growth factor receptor comprising providing an effective amount of a compound of any of Formula IIIa, IIIaa, IIIb or IIIbb wherein R₁ is a moiety having the structure —(CHR₁ₐ)_{z}—R_{1b}, wherein z is a number selected from the group consisting of 0, 1, 2 and 3; R₁ₐ is a moiety selected from the group consisting of H, (C₁-C₄)alkyl, F, (C₁-C₄)fluoroalkyl, (C₁-C₄)alkoxy, —(C₁-C₄)alkylamine, —(C₁-C₄)dialkylamine, —C(O)OH, —C(O)—NH₂, —C(O)—(C₁-C₄)alkyl, —C(O)—(C₁-C₄)fluoralkyl, —C(O)—(C₁-C₄)alkylamine, and —C(O)—(C₁-C₄)alkoxy; and R_{1b} is a moiety selected from the group consisting of —(C₁-C₄)alkyl, an optionally substituted —(C₃-C₆)cycloalkyl, —(C₁-C₄)fluoroalkyl, and an optionally substituted 5-membered or 6-membered unsaturated heterocycle; or R_{1b} is H when z is 1, 2, or 3, are provided herein.

Provided herein are compositions, methods of treating, and methods for modulating the activity of epidermal growth factor receptor comprising providing an effective amount of a compound of any of Formula IIIa, IIIaa, IIIb or IIbb wherein R₃ is —(CHR₃ₐ)ₓ—R_{3b}, wherein x is 0, 1, 2, or 3; and R₃ₐ is selected from the group consisting of H, (C₁-C₄)alkyl, F, (C₁-C₄)fluoroalkyl, (C₁-C₄)alkoxy, —(C₁-C₄)alkylamine, and —(C₁-C₄)dialkylamine; and R_{3b} is H or a phenyl, optionally substituted with 1-2 substituents independently selected from the group consisting of halogen, —(C₁-C₄)alkyl, —(C₁-C₄)fluoroalkyl, —(C₁-C₄)alkoxy, —(C₁-C₄)alkylamine, and —(C₁-C₄)dialkylamine, are also provided herein. In some embodiments, R₃ is —NH—(CHR₃ₐ)ₓ—R_{3b}, wherein x is 0, 1, 2, or 3; and R₃ₐ is selected from the group consisting of H, (C₁-C₄)alkyl, F, (C₁-C₄)fluoroalkyl, (C₁-C₄)alkoxy, —(C₁-C₄)alkylamine, and —(C₁-C₄)dialkylamine; and R_{3b} is H or a phenyl, optionally substituted with 1-2 substituents independently selected from the group consisting of halogen, —(C₁-C₄)alkyl, —(C₁-C₄)fluoroalkyl, —(C₁-C₄)alkoxy, —(C₁-C₄)alkylamine, and —(C₁-C₄)dialkylamine. In other embodiments, R₃ is —O—(CHR₃ₐ)ₓ—R_{3b}, wherein x is 0, 1, 2, or 3; and R₃ₐ is selected from the group consisting of H, (C₁-C₄)alkyl, F, (C₁-C₄)fluoroalkyl, (C₁-C₄)alkoxy, —(C₁-C₄)alkylamine, and —(C₁-C₄)dialkylamine; and R_{3b} is H or a phenyl, optionally substituted with 1-2 substituents independently selected from the group consisting of halogen, —(C₁-C₄)alkyl, —(C₁-C₄)fluoroalkyl, —(C₁-C₄)alkoxy, —(C₁-C₄)alkylamine, and —(C₁-C₄)dialkylamine. In still other embodiments, R₃ is —S—(CHR₃ₐ)ₓ—R_{3b}, wherein x is 0, 1, 2, or 3; and R₃ₐ is selected from the group consisting of H, (C₁-C₄)alkyl, F, (C₁-C₄)fluoroalkyl, (C₁-C₄)alkoxy, —(C₁-C₄)alkylamine, and —(C₁-C₄)dialkylamine; and R_{3b} is H or a phenyl, optionally substituted with 1-2 substituents independently selected from the group consisting of halogen, —(C₁-C₄)alkyl, —(C₁-C₄)fluoroalkyl, —(C₁-C₄)alkoxy, —(C₁-C₄)alkylamine, and —(C₁-C₄)dialkylamine.

Provided herein are compositions, methods of treating, and methods for modulating the activity of epidermal growth factor receptor comprising providing an effective amount of a compound of any of Formula IV or IVa: 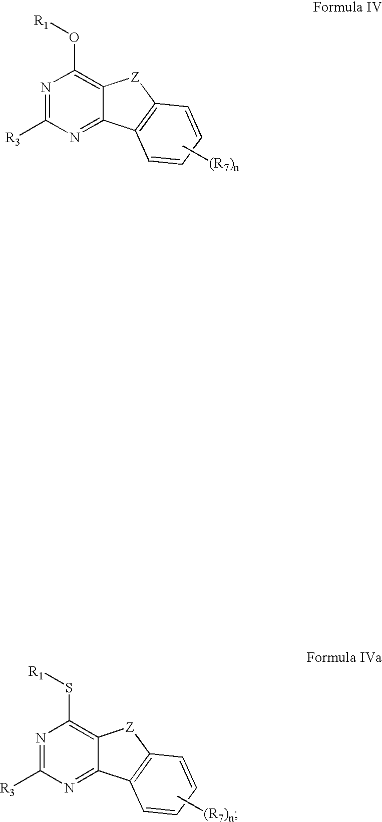
wherein Z is selected from the group consisting of O, S, and NR₄;
(a) R₁ is selected from one of the following options:
   a. R₁ is a moiety having the structure —(CHR₁ₐ)_{z}—R_{1b},
      i. wherein z is a number selected from the group consisting of 0, 1, 2 and 3;
      ii. R₁ₐ is a moiety selected from the group consisting of H, (C₁-C₄)alkyl, F, (C₁-C₄)fluoroalkyl, (C₁-C₄)alkoxy, —C(O)OH, —C(O)—NH₂, —C(O)—(C₁-C₄)alkyl, —C(O)—(C₁-C₄)fluoralkyl, —C(O)—(C₁-C₄)alkylamine, and —C(O)—(C₁-C₄)alkoxy; and
      iii. R_{1b} is phenyl, optionally substituted with 1-4 moieties independently selected from the group consisting of halogen, —CN, -L₁-OH, -L₁-NH₂, -L₁-(C₁-C₄)alkyl, -L₁-(C₃-C₆)cycloalkyl, -L₁-(C₁-C₄)fluoroalkyl, -L₁-(C₁-C₄)alkoxy, -L₁-(C₁-C₄)alkylamine, -L₁-(C₁-C₄)dialkylamine and -L₁-phenyl, wherein L₁ is a bond, —C(O)— and S(O)₂; or
   b. R₁ is a moiety having the structure —(CHR₁ₐ)_{z}—R_{1b},
      i. wherein z is a number selected from the group consisting of 0, 1, 2 and 3;
      ii. R₁ₐ is a moiety selected from the group consisting of H, (C₁-C₄)alkyl, F, (C₁-C₄)fluoroalkyl, (C₁-C₄)alkoxy, —(C₁-C₄)alkylamine, —(C₁-C₄)dialkylamine, —C(O)OH, —C(O)—NH₂, —C(O)—(C₁-C₄)alkyl, —C(O)—(C₁-C₄)fluoralkyl, —C(O)—(C₁-C₄)alkylamine, and —C(O)—(C₁-C₄)alkoxy; and
      iii. R_{1b} is a moiety selected from the group consisting of —(C₁-C₄)alkyl, an optionally substituted —(C₃-C₆)cycloalkyl, —(C₁-C₄)fluoroalkyl, and an optionally substituted 5-membered or 6-membered unsaturated heterocycle; or R_{1b} is H when z is 1, 2, or 3;
(b) n is 0, 1, 2, or 3; and each R₇ is independently selected from the group consisting of halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, —(C₁-C₄)dialkylamine, —C(O)OH, —C(O)—NH₂, —C(O)—(C₁-C₄)alkyl, —C(O)—(C₁-C₄)fluoralkyl, —C(O)—(C₁-C₄)alkylamine, and —C(O)—(C₁-C₄)alkoxy;
   or a pharmaceutically acceptable salt, pharmaceutically acceptable N-oxide, pharmaceutically active metabolite, pharmaceutically acceptable prodrug, or pharmaceutically acceptable solvate thereof.

Provided herein are compositions, methods of treating, and methods for modulating the activity of epidermal growth factor receptor comprising providing an effective amount of a compound of any of Formula IV or IVa wherein R₁ is a moiety having the structure —(CHR₁ₐ)_{z}—R_{1b}, wherein z is a number selected from the group consisting of 0, 1, 2 and 3; R₁ₐ is a moiety selected from the group consisting of H, (C₁-C₄)alkyl, F, (C₁-C₄)fluoroalkyl, (C₁-C₄)alkoxy, —C(O)OH, —C(O)—NH₂, —C(O)—(C₁-C₄)alkyl, —C(O)—(C₁-C₄)fluoralkyl, —C(O)—(C₁-C₄)alkylamine, and —C(O)—(C₁-C₄)alkoxy; and R_{1b} is phenyl, optionally substituted with 1-4 moieties independently selected from the group consisting of halogen, —CN, -L₁-OH, -L₁-NH₂, -L₁-(C₁-C₄)alkyl, -L₁-(C₃-C₆)cycloalkyl, -L₁-(C₁-C₄)fluoroalkyl, -L₁-(C₁-C₄)alkoxy, -L₁-(C₁-C₄)alkylamine, -L₁-C₁-C₄)dialkylamine and -L₁-phenyl, wherein L₁ is a bond, —C(O)— and S(O)₂, are provided herein. In some embodiments, z is 0; or z is 1 and R₁ₐ is a moiety selected from the group consisting of H and (C₁-C₄)alkyl. In other embodiments, R₁ is a moiety having the structure —(CHR₁ₐ)_{z}—R_{1b}, wherein z is a number selected from the group consisting of 0, 1, 2 and 3; R₁ₐ is a moiety selected from the group consisting of H, (C₁-C₄)alkyl, F, (C₁-C₄)fluoroalkyl, (C₁-C₄)alkoxy, —(C₁-C₄)alkylamine, —(C₁-C₄)dialkylamine, —C(O)OH, —C(O)—NH₂, —C(O)—(C₁-C₄)alkyl, —C(O)—(C₁-C₄)fluoralkyl, —C(O)—(C₁-C₄)alkylamine, and —C(O)—(C₁-C₄)alkoxy; and R_{1b} is a moiety selected from the group consisting of —(C₁-C₄)alkyl, an optionally substituted —(C₃-C₆)cycloalkyl, —(C₁-C₄)fluoroalkyl, and an optionally substituted 5-membered or 6-membered unsaturated heterocycle; or R_{1b} is H when z is 1, 2, or 3.

Provided herein are compositions, methods of treating, and methods for modulating the activity of epidermal growth factor receptor comprising providing an effective amount of a compound of any of Formula IV or IVa wherein R₃ is —(CHR₃ₐ)ₓ—R_{3b}, wherein x is 0, 1, 2, or 3; and R₃ₐ is selected from the group consisting of H, (C₁-C₄)alkyl, F, (C₁-C₄)fluoroalkyl, (C₁-C₄)alkoxy, —(C₁-C₄)alkylamine, and —(C₁-C₄)dialkylamine; and R_{3b} is H or a phenyl, optionally substituted with 1-2 substituents independently selected from the group consisting of halogen, —(C₁-C₄)alkyl, —(C₁-C₄)fluoroalkyl, —(C₁-C₄)alkoxy, —(C₁-C₄)alkylamine, and —(C₁-C₄)dialkylamine. In some embodiments, R₃ is hydrogen. In other embodiments, R₃ is —NH—(CHR₃ₐ)ₓ—R_{3b}, wherein x is 0, 1, 2, or 3; and R₃ₐ is selected from the group consisting of H, (C₁-C₄)alkyl, F, (C₁-C₄)fluoroalkyl, (C₁-C₄)alkoxy, —(C₁-C₄)alkylamine, and —(C₁-C₄)dialkylamine; and R_{3b} is H or a phenyl, optionally substituted with 1-2 substituents independently selected from the group consisting of halogen, —(C₁-C₄)alkyl, —(C₁-C₄)fluoroalkyl, —(C₁-C₄)alkoxy, —(C₁-C₄)alkylamine, and —(C₁-C₄)dialkylamine. In yet other embodiments, R₃ is —O—(CHR₃ₐ)ₓ—R_{3b}, wherein x is 0, 1, 2, or 3; and R₃ₐ is selected from the group consisting of H, (C₁-C₄)alkyl, F, (C₁-C₄)fluoroalkyl, (C₁-C₄)alkoxy, —(C₁-C₄)alkylamine, and —(C₁-C₄)dialkylamine; and R_{3b} is H or a phenyl, optionally substituted with 1-2 substituents independently selected from the group consisting of halogen, —(C₁-C₄)alkyl, —(C₁-C₄)fluoroalkyl, —(C₁-C₄)alkoxy, —(C₁-C₄)alkylamine, and —(C₁-C₄)dialkylamine. In still other embodiments, R₃ is —S—(CHR₃ₐ)ₓ—R_{3b}, wherein x is 0, 1, 2, or 3; and R₃ₐ is selected from the group consisting of H, (C₁-C₄)alkyl, F, (C₁-C₄)fluoroalkyl, (C₁-C₄)alkoxy, —(C₁-C₄)alkylamine, and —(C₁-C₄)dialkylamine; and R_{3b} is H or a phenyl, optionally substituted with 1-2 substituents independently selected from the group consisting of halogen, —(C₁-C₄)alkyl, —(C₁-C₄)fluoroalkyl, —(C₁-C₄)alkoxy, —(C₁-C₄)alkylamine, and —(C₁-C₄)dialkylamine.

In some embodiments, the method involving the use of compounds having the structure of any of Formula 1, Formula 2, Formula 3, Formula 4, or Formula 5 comprises contacting the epidermal growth factor receptor with an effective amount of the compound. In other embodiments, the contacting occurs in vivo. In other embodiments, the contacting occurs within a human patient, wherein the human patient has an EGFR-mediated disease or condition. In various embodiments, the effective amount is an amount effective for treating an EGFR-mediated disease or condition within the body of the person. In some embodiments the EGFR-mediated disease or condition is selected from the group consisting of blood vessel growth, cancer, benign hyperplasia, keloid formation, and psoriasis.

In certain embodiments, isomers, diastereomers, enantiomers, metabolites, prodrugs, salts, or esters of the compounds described herein are administered to the patient. In certain embodiments involving the use of compounds having the structure of any of Formula 1, Formula 2, Formula 3, Formula 4, or Formula 5, the conditions or diseases are associated with at least one kinase activity, in further embodiments the conditions or diseases are associated with at least one protein tyrosine kinase activity, in further embodiments the conditions or diseases are associated with at least one receptor tyrosine kinase activity, in further embodiments the conditions or diseases are associated with at least one activity of a kinase in the HER subfamily of receptor tyrosine kinases, and in further embodiments the conditions or diseases are associated with EGFR activity. In some embodiments, the kinase is a class III receptor tyrosine kinase (RTKIII). In other embodiments, the kinase is a tyrosine kinase receptor intimately involved in the regulation and stimulation of cellular proliferation. In some embodiments, the compounds disclosed herein directly inhibit EGFR activity. In other embodiments, the compounds disclosed herein indirectly inhitit EGFR activity. As used herein, EGFR activity includes the activity of one or more of the tyrosine kinase activities of EGFR, such as ErbB2, ErbB3, or ErbB4.

In one aspect are methods for treating a disease comprising administering to a subject in need thereof an effective amount of an epidermal growth factor receptor modulating compound corresponding to Formula (I): 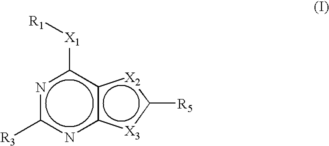
wherein:
a. X₁ is S or O;
b. each of X₂ and X₃ is independently N, O, S, NR₄, or CR₆;
c. R₁ is —(CHR₁ₐ)_{z}—R_{1b}, where
   i. each R₁ₐ is independently H, (C₁-C₄)alkyl, F, (C₁-C₄)fluoroalkyl, (C₁-C₄)alkoxy, —C(O)OH, —C(O)—NH₂, —C(O)—(C₁-C₄)alkyl, —C(O)—(C₁-C₄)fluoralkyl, —C(O)—(C₁-C₄)alkyl amine, —(C₁-C₄)alkylamine, —(C₁-C₄)dialkylamine, or —C(O)—(C₁-C₄)alkoxy,
   ii. z is 0, 1, 2, or 3, and
   iii. R_{1b} is 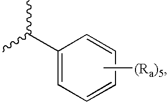
      where each Rₐ is independently H, halogen, substituted or unsubstituted alkyl, substituted or unsubstituted alkoxy, —CN, -L₁-OH, -L₁-NH₂, -L₁-(C₁-C₄)alkyl, -L₁-(C₃-C₆)cycloalkyl, -L₁-(C₁-C₄)fluoroalkyl, -L₁-(C₁-C₄)alkoxy, -L₁-(C₁-C₄)alkylamine, -L₁-(C₁-C₄)dialkylamine and -L₁-phenyl, wherein L₁ is a bond, —C(O)—, or —S(O)₂—; or
   R_{1b} is H, —(C₁-C₄)alkyl, an optionally substituted —(C₃-C₆)cycloalkyl, —(C₁-C₄)fluoroalkyl, or an optionally substituted 5-membered or 6-membered unsaturated heterocycle;
d. R₃ is H or L₃-(CHR₃ₐ)ₓ—R_{3b}, where
   i. L₃ is a bond, NH, O, or S,
   ii. R₃ₐ is H, (C₁-C₄)alkyl, F, (C₁-C₄)fluoroalkyl, (C₁-C₄)alkoxy, —(C₁-C₄)alkylamine, or —(C₁-C₄)dialkylamine,
   iii. x is 0, 1, 2, or 3, and
   iv. R_{3b} is phenyl, optionally substituted with 1-2 substituents independently selected from the group consisting of halogen, —(C₁-C₄)alkyl, —(C₁-C₄)fluoroalkyl, —(C₁-C₄)alkoxy, —(C₁-C₄)alkylamine, and —(C₁-C₄)dialkylamine;
e. R₄ is H or —(CHR₄ₐ)_{y}—R_{4b}, where
   i. R₄ₐ is H, (C₁-C₄)alkyl, F, (C₁-C₄)fluoroalkyl, (C₁-C₄)alkoxy, —(C₁-C₄)alkylamine, or —(C₁-C₄)dialkylamine;
   ii. y is 0, 1, 2, or 3, and
   iii. R_{4b} is substituted or unsubstituted alkyl, substituted or unsubstituted cycloalkyl, substituted or unsubstituted phenyl, or substituted or unsubstituted 5-membered or 6-membered unsaturated heterocycle; or
R₄ and R₅, taken together, form a 5- or 6-membered heterocyclic aromatic ring structure, optionally substituted with 1-2 moieties independently selected from the group consisting of halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, and —(C₁-C₄)dialkylamine; or
when X₂ is NR₄ and X₃ is CR₆, R₁ and R₄, taken together, form a 5- or 6-membered aromatic heterocycle optionally substituted with 1-2 moieties independently selected from the group consisting of halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, and —(C₁-C₄)dialkylamine; or
f. R₅ is H or 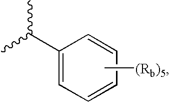
   where each R_{b} is independently H, halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, —(C₁-C₄)dialkylamine, —C(O)OH, —C(O)—NH₂, —C(O)—(C₁-C₄)alkyl, —C(O)—(C₁-C₄)fluoralkyl, —C(O)—(C₁-C₄)alkylamine, or —C(O)—(C₁-C₄)alkoxy; and
g. R₆ is H, heteroaryl, or phenyl, wherein the phenyl and the heteroaryl are optionally substituted with 1-2 moieties independently selected from the group consisting of halogen, —(C₁-C₄)alkyl, —(C₁-C₄)fluoroalkyl, —(C₁-C₄)alkoxy, —(C₁-C₄)alkylamine, and —(C₁-C₄)dialkylamine; or
   R₆ and R₅, taken together, form an aromatic carbocycle or heterocycle optionally substituted with 1-2 moieties independently selected from the group consisting of halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, and —(C₁-C₄)dialkylamine, or
   when X₂ is CR₆ and X₃ is NR₄, R₆ and R₁, taken together, form a 5- or 6-membered aromatic heterocycle optionally substituted with 1-2 moieties independently selected from the group consisting of halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, and —(C₁-C₄)dialkylamine;
   or a pharmaceutically acceptable salt, pharmaceutcaly acceptable N-oxide, pharmaceutically acitve metabolite, pharmaceutically acceptable prodrug, or pharmaceutically acceptable solvate thereof.

In another or further embodiment, R₁ of said compound is 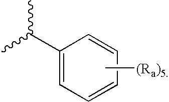
In another or further embodiment, each Rₐ of said compound is independently H, halogen, (C₁-C₄)alkyl, or (C₁-C₄)alkoxy. In another or further embodiment, R₃ of said compound is H. In another or further embodiment, R₅ of said compound is H or 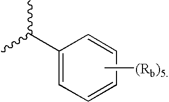
In another or further embodiment, each R_{b} of said compound is independently H, halogen, (C₁-C₄)alkyl, (C₁-C₄)alkoxy, or —OH.

In another or further embodiment, X₁ of said compound is S. In another or further embodiment, X₁ of said compound is O. In another or further embodiment, X₂ of said compound is CR₆ and X₃ of said compound is NR₄. In another or further embodiment, X₂ of said compound is CR₆ and X₃ of said compound is O. In another or further embodiment, X₂ of said compound is CR₆ and X₃ of said compound is S. In another or further embodiment, X₂ of said compound is N and X₃ of said compound is NR₄.

In another or further embodiment, R₄ of said compound is H or (C₁-C₄)alkyl. In another or further embodiment, R₆ of said compound is H. In another or further embodiment, each of R₆ and R₃ of said compound is H.

In another or further embodiment, said compound corresponds to Formula (Ia-O): 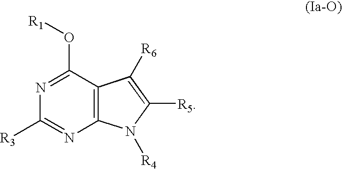
In another or further embodiment, said compound corresponds to Formula (Ia-S):

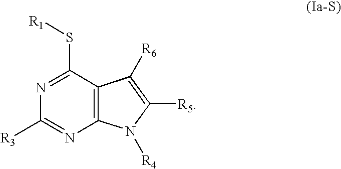

In another or further embodiment, said compound corresponds to Formula (Ib-O): 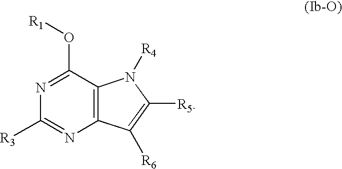
In another or further embodiment, said compound corresponds to Formula (Ib-S):

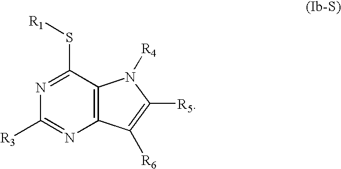

In another or further embodiment, said compound corresponds to Formula (IIa- 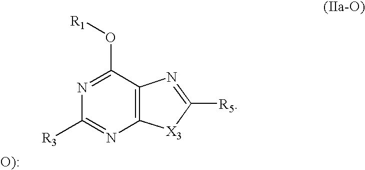
In another or further embodiment, X₃ of said compound corresponding to Formula (IIa) is O, S, or NR₄.

In another or further embodiment, said compound corresponds to Formula (IIa-S): 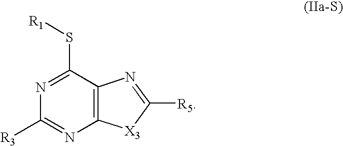
In another or further embodiment, X₃ of said compound corresponding to Formula (IIa-S) is O, S, or NR₄.

In another or further embodiment, said compound corresponds to Formula (IIb- 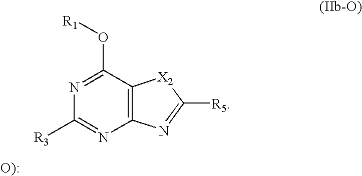
In another or further embodiment, X₃ of said compound corresponding to Formula (IIb) is O, S, or NR₄.

In another or further embodiment, said compound corresponds to Formula (IIb-S): 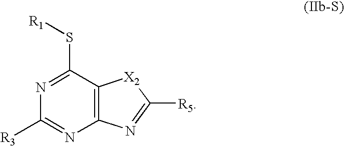
In another or further embodiment, X₃ of said compound corresponding to Formula (IIa) is O, S, or NR₄.

In another or further embodiment, said compound corresponds to Formula (IIIa- 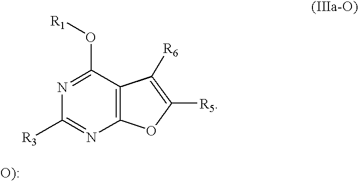
In another or further embodiment, said compound corresponds to Formula (IIIa-S):

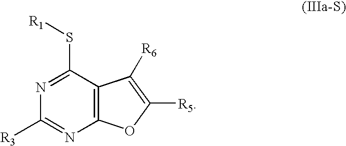

In another or further embodiment, said compound corresponds to Formula (IIIb- 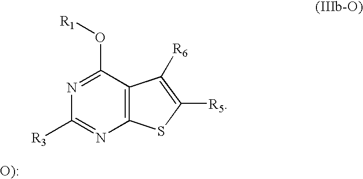
In another or further embodiment, said compound corresponds to Formula (IIIb-S):

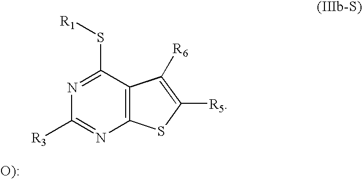

In another or further embodiment, said compound corresponds to Formula (A1): 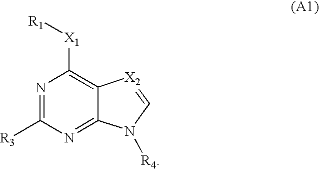
In another or further embodiment, X₂ of said compound corresponding to Formula (A1) is N or CR₆. In another or further embodiment, said compound is selected from the group consisting of:

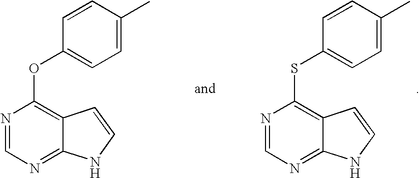

In another or further embodiment, said compound corresponds to Formula (A): 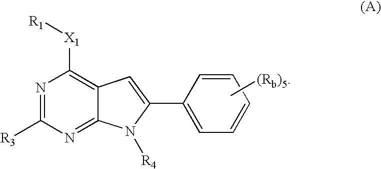
In another or further embodiment, said compound corresponds to Formula (B): 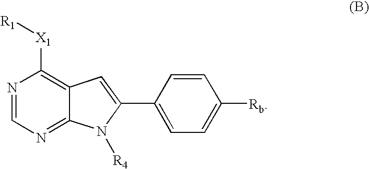
In another or further embodiment, said compound corresponds to Formula (C):

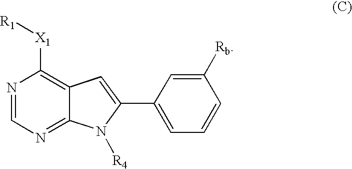

In another or further embodiment, said compound corresponds to Formula (D): 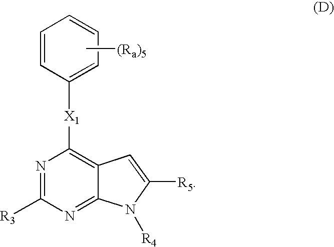
In another or further embodiment, the compound corresponds to Formula (E): 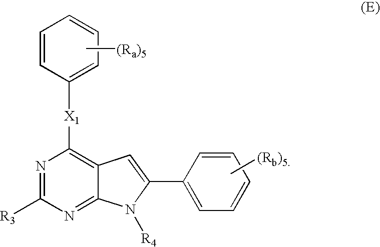
In another or further embodiment, said compound is selected from the group consisting of: 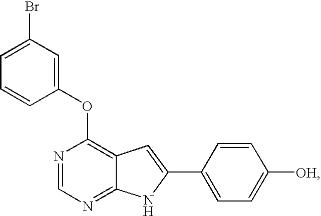
said compound corresponds to Formula (H): 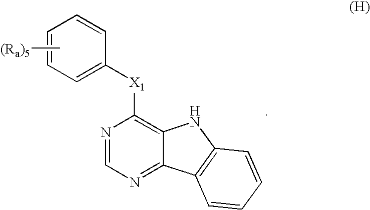
In another or further embodiment, said compound is selected from the group consisting of:

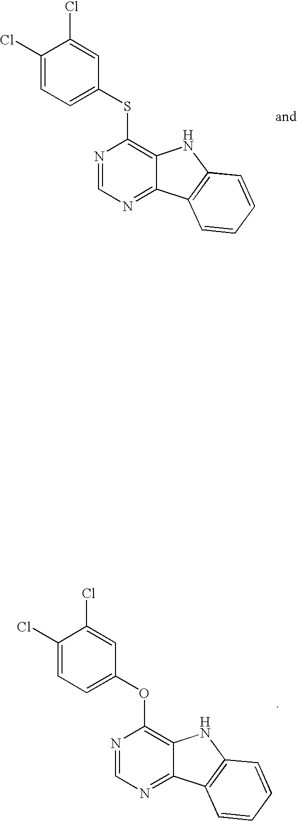

In another aspect are methods for treating a disease comprising administering to a subject in need thereof an effective amount of an EGFR kinase modulating compound corresponding to Formula (I): 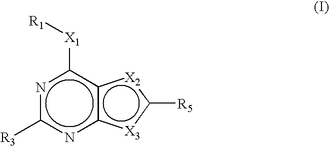
wherein:
a. X₁ is S or O;
b. each of X₂ and X₃ is independently N, O, S, NR₄, or CR₆;
c. R₁ₐ is —(CHR₁ₐ)_{z}—R_{1b}, where
   i. each R₁ₐ is independently H, (C₁-C₄)alkyl, F, (C₁-C₄)fluoroalkyl, (C₁-C₄)alkoxy, —C(O)OH, —C(O)—NH₂, —C(O)—(C₁-C₄)alkyl, —C(O)—(C₁-C₄)fluoralkyl, —C(O)—(C₁-C₄)alkylamine, —(C₁-C₄)alkylamine, —(C₁-C₄)dialkylamine, or —C(O)—(C₁-C₄)alkoxy,
   ii. z is 0, 1, 2, or 3, and
   iii. R_{1b} is

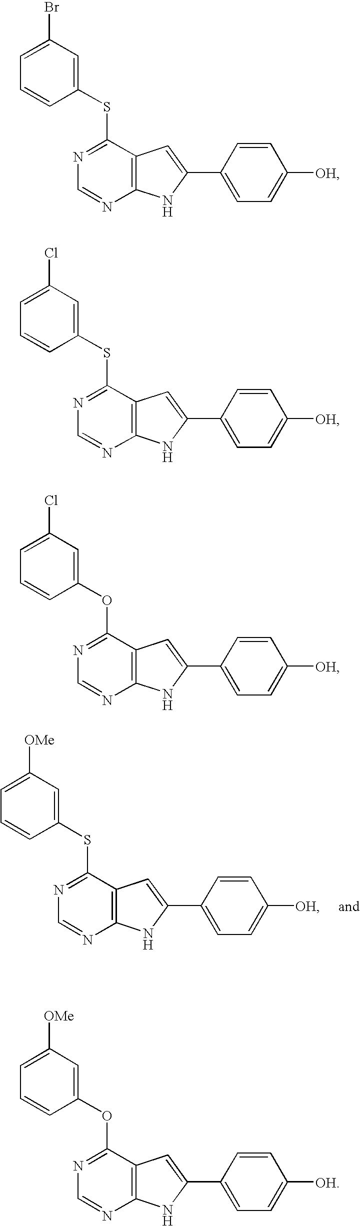

In another or further embodiment, X₂ is NR₄ and X₃ is CR₆. In another or further embodiment, R₅ and R₆ are taken together to form an optionally substituted phenyl ring.

In another or further embodiment, said compound corresponds to Formula (IV): 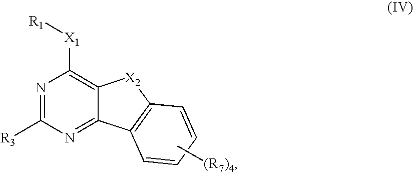
wherein
X₂ is O, S, or NR₄; and
each R₇ is independently selected from the group consisting of H, halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, —(C₁-C₄)dialkylamine, —C(O)OH, —C(O)—NH₂, —C(O)—(C₁-C₄)alkyl, —C(O)—(C₁-C₄)fluoralkyl, —C(O)—(C₁-C₄)alkylamine, and —C(O)—(C₁-C₄)alkoxy.

In another or further embodiment, said compound corresponds to Formula (F): 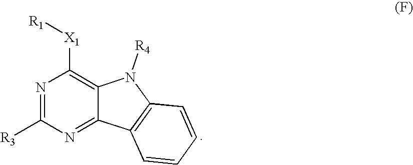
In another or further embodiment, said compound corresponds to Formula (G): 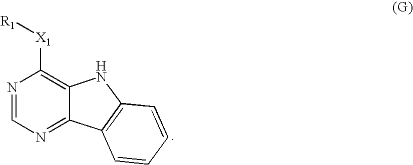
In another or further embodiment, 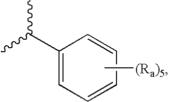
where each Rₐ is independently H, halogen, substituted or unsubstituted alkyl, substituted or unsubstituted alkoxy, —CN, -L₂-OH, -L₁-NH₂, -L₁-(C₁-C₄)alkyl, -L₁-(C₃-C₆)cycloalkyl, -L₁-(C₁-C₄)fluoroalkyl, -L₁-(C₁-C₄)alkoxy, -L₁-(C₁-C₄)alkylamine, -L₁-(C₁-C₄)dialkylamine and -L₁-phenyl, wherein L₁ is a bond, —C(O)—, or —S(O)₂—; or
R_{1b} is H, —(C₁-C₄)alkyl, an optionally substituted —(C₃-C₆)cycloalkyl, —(C₁-C₄)fluoroalkyl, or an optionally substituted 5-membered or 6-membered unsaturated heterocycle;
d. R₃ is H or L₃-(CHR₃ₐ)ₓ—R_{3b}, where
   i. L₃ is a bond, NH, O, or S,
   ii. R₃ₐ is H, (C₁-C₄)alkyl, F, (C₁-C₄)fluoroalkyl, (C₁-C₄)alkoxy, —(C₁-C₄)alkylamine, or —(C₁-C₄)dialkylamine,
   iii. x is 0, 1, 2, or 3, and
   iv. R_{3b} is phenyl, optionally substituted with 1-2 substituents independently selected from the group consisting of halogen, —(C₁-C₄)alkyl, —(C₁-C₄)fluoroalkyl, —(C₁-C₄)alkoxy, —(C₁-C₄)alkylamine, and —(C₁-C₄)dialkylamine;
e. R₄ is H or —(CHR₄ₐ)_{y}—R_{4b}, where
   i. R₄ₐ is H, (C₁-C₄)alkyl, F, (C₁-C₄)fluoroalkyl, (C₁-C₄)alkoxy, —(C₁-C₄)alkylamine, or —(C₁-C₄)dialkylamine;
   ii. y is 0, 1, 2, or 3, and
   iii. R_{4b} is substituted or unsubstituted alkyl, substituted or unsubstituted cycloalkyl, substituted or unsubstituted phenyl, or substituted or unsubstituted 5-membered or 6-membered unsaturated heterocycle; or
R₄ and R₅, taken together, form a 5- or 6-membered heterocyclic aromatic ring structure, optionally substituted with 1-2 moieties independently selected from the group consisting of halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, and —(C₁-C₄)dialkylamine; or
when X₂ is NR₄ and X₃ is CR₆, R₁ and R₄, taken together, form a 5- or 6-membered aromatic heterocycle optionally substituted with 1-2 moieties independently selected from the group consisting of halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, and —(C₁-C₄)dialkylamine; or
f. R₅ is H or 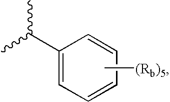
   where each R_{b} is independently H, halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, —(C₁-C₄)dialkylamine, —C(O)OH, —C(O)—NH₂, —C(O)—(C₁-C₄)alkyl, —C(O)—(C₁-C₄)fluoralkyl, —C(O)—(C₁-C₄)alkylamine, or —C(O)—(C₁-C₄)alkoxy; and
g. R₆ is H, heteroaryl, or phenyl, wherein the phenyl and the heteroaryl are optionally substituted with 1-2 moieties independently selected from the group consisting of halogen, —(C₁-C₄)alkyl, —(C₁-C₄)fluoroalkyl, —(C₁-C₄)alkoxy, —(C₁-C₄)alkylamine, and —(C₁-C₄)dialkylamine; or
   R₆ and R₅, taken together, form an aromatic carbocycle or heterocycle optionally substituted with 1-2 moieties independently selected from the group consisting of halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, and —(C₁-C₄)dialkylamine, or
   when X₂ is CR₆ and X₃ is NR₄, R₆ and R₁, taken together, form a 5- or 6-membered aromatic heterocycle optionally substituted with 1-2 moieties independently selected from the group consisting of halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, and —(C₁-C₄)dialkylamine;
   or a pharmaceutically acceptable salt, pharmaceutcaly acceptable N-oxide, pharmaceutically acitve metabolite, pharmaceutically acceptable prodrug, or pharmaceutically acceptable solvate thereof.

In another or further embodiment, the contacting occurs in vivo. In another or further embodiment, the contacting occurs within a human patient, wherein the human patient has an EGFR-mediated disease or condition. In another or further embodiment, the effective amount is an amount effective for treating an EGFR-mediated disease or condition within the body of the person. In another or further embodiment, the EGFR-mediated disease or condition is selected from the group consisting of blood vessel growth, cancer, benign hyperplasia, keloid formation, and psoriasis.

In another aspect are methods for treating a disease comprising administering to a subject in need thereof an effective amount of an epidermal growth factor receptor modulating compound corresponding to: 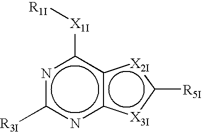
wherein:
a. X_{1I} is S or O;
b. each of X_{2I} and X_{3I} is independently N, O, S, NR_{4I}, or CR_{6I};
c. R_{1I} is —(CHR_{1aI})_{zI}—R_{1bI}, where
   i. each R_{1aI} is independently H, halogen or a substituted or unsubstituted moiety selected from alkyl, haloalkyl, heteroalkyl, cycloalkyl, heterocycloalkyl, alkenyl, alkynyl, alkoxy, alkylamine, dialkylamine, —C(O)OH, —C(O)NH₂, —C(O)-alkyl, —C(O)-haloalkyl, —C(O)-alkylamine, and —C(O)-alkoxy,
   ii. z_{I} is 0, 1, 2, 3, or 4 and
   iii. R_{1bI} is 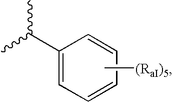
      where each R_{aI} is independently H, halogen, —CN, —OH, or a substituted or unsubstituted moiety selected from the group consisting of alkyl, alkoxy, haloalkyl, alkenyl, alkynyl, heteroalkyl, -L₁-OH, -L₁-NH₂, -L₁-alkyl, -L₁-cycloalkyl, -L₁-haloalkyl, -L₁-alkoxy, -L₁-alkylamine, -L₁-dialkylamine and -L₁-phenyl, wherein L₁ is a bond, —C(O)—, or —S(O)₂—; or
   R_{1bI} is H, alkyl, or a substituted or unsubstituted moiety selected from cycloalkyl, haloalkyl, and heterocycle;
d. R_{3I} is H or L_{3I}-(CHR_{3aI})ₓ—R_{3bI}, where
   i. L_{3I} is a bond, NH, O, or S,
   ii. R_{3aI} is H, alkyl, halogen, haloalkyl, alkoxy, alkylamine, or dialkylamine,
   iii. x_{I} is 0, 1, 2, 3, or 4 and
   iv. R_{3bI} is H or substituted or unsubstituted aryl or heteroaryl group;
e. R_{4I} is H or —(CHR_{4aI})_{yI}—R_{4bI}, where
   i. R_{4aI} is H, alkyl, halogen, haloalkyl, alkoxy, alkylamine, or dialkylamine;
   ii. y_{I} is 0, 1, 2, 3, or 4 and
   iii. R_{4bI} is a substituted or unsubstituted moiety selected from alkyl, cycloalkyl, heterocycloalkyl, aryl, and heteroaryl; or
R_{4I} and R_{5I}, taken together, form a substituted or unsubstitued heteroaryl moiety; or
when X_{1I} is NR_{4I} and X_{2I} is CR_{6I}, R_{1I} and R_{4I}, taken together, form a substituted or
   unsubstituted heterocycle; or
f. R_{5I} is H or 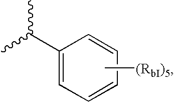
   where each R_{bI} is independently H, halogen, —CN, —OH, —NH₂, or a substituted or unsubstituted moiety selected from alkyl, cycloalkyl, haloalkyl, alkoxy, alkylamine, dialkylamine, —C(O)OH, —C(O)—NH₂, —C(O)-alkyl, —C(O)-haloalkyl, —C(O)-alkylamine, and —C(O)-alkoxy; and
g. R_{6I} is H, substituted or unsubstituted heteroaryl, or substituted or unsubstituted aryl; or
   R_{6I} and R_{5I}, taken together, form a substituted or unsubstituted aryl or heteroaryl moiety, or
   when X_{1I} is CR_{6I} and X_{2I} is NR_{4I}, R_{6I} and R_{1I}, taken together, form a substituted or unsubstituted heterocycle;
   or a pharmaceutically acceptable salt, pharmaceutcaly acceptable N-oxide, pharmaceutically acitve metabolite, pharmaceutically acceptable prodrug, or pharmaceutically acceptable solvate thereof.

In another or further embodiment, the disease is selected from the group consisting of blood vessel growth, cancer, benign hyperplasia, keloid formation, and psoriasis.

Compositions described herein may be administered in a pharmaceutical composition containing one or more pharmaceutically acceptable excipients suitable. In some embodiments, the composition is in the form of a tablet, a capsule, or a soft-gel capsule. In other embodiments, the excipient is a liquid suited for administration by injection, including intravenous, intramuscular, or subcutaneous administration. And, in yet other embodiments, the excipient is suited to topical, transdermal, or buccal administration, or as a suppository.

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise. Definition of standard chemistry terms may be found in reference works, including Carey and Sundberg (1992) “ADVANCED ORGANIC CHEMISTRY 3^{RD} ED.” Vols. A and B, Plenum Press, New York. Unless otherwise indicated, conventional methods of mass spectroscopy, NMR, HPLC, protein chemistry, biochemistry, recombinant DNA techniques and pharmacology, within the skill of the art are employed.

The term “agonist” means a molecule such as a compound, a drug, an enzyme activator or a hormone that enhances the activity of another molecule or the activity of a receptor site.

The term “alkenyl group” includes a monovalent unbranched or branched hydrocarbon chain having one or more double bonds therein. The double bond of an alkenyl group can be unconjugated or conjugated to another unsaturated group. Suitable alkenyl groups include, but are not limited to, (C₂-C₈)alkenyl groups, such as vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl. An alkenyl group can be unsubstituted or substituted.

The term “alkoxy” as used herein includes —O-(alkyl), wherein alkyl is defined herein.

The term “alkyl” means a straight chain or branched, saturated or unsaturated chain having from 1 to 10 carbon atoms. Representative saturated alkyl groups include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, n-pentyl, isopentyl, neopentyl, and n-hexyl, and longer alkyl groups, such as heptyl, and octyl. An alkyl group can be unsubstituted or substituted. Unsaturated alkyl groups include alkenyl groups and alkynyl groups, discussed herein. Alkyl groups containing three or more carbon atoms may be straight, branched or cyclized.

The term “alkynyl group” includes a monovalent unbranched or branched hydrocarbon chain having one or more triple bonds therein. The triple bond of an alkynyl group can be unconjugated or conjugated to another unsaturated group. Suitable alkynyl groups include, but are not limited to, (C₂-C₆)alkynyl groups, such as ethynyl, propynyl, butynyl, pentynyl, hexynyl, methylpropynyl, 4-methyl-1-butynyl, 4-propyl-2-pentynyl, and 4-butyl-2-hexynyl. An alkynyl group can be unsubstituted or substituted.

The term “antagonist” means a molecule such as a compound, a drug, an enzyme inhibitor, or a hormone, that diminishes or prevents the action of another molecule or the activity of a receptor site.

The term “aryl” includes a carbocyclic or heterocyclic aromatic group containing from 5 to 30 ring atoms. The ring atoms of a carbocyclic aromatic group are all carbon atoms, and include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. A carbocyclic aromatic group can be unsubstituted or substituted. Preferably, the carbocyclic aromatic group is a phenyl group. The ring atoms of a heterocyclic aromatic group contains at least one heteroatom, preferably 1 to 3 heteroatoms, independently selected from nitrogen, oxygen, and sulfur. Illustrative examples of heterocyclic aromatic groups include, but are not limited to, pyridinyl, pyridazinyl, pyrimidyl, pyrazyl, triazinyl, pyrrolyl, pyrazolyl, imidazolyl, (1,2,3,)- and (1,2,4)-triazolyl, pyrazinyl, pyrimidinyl, tetrazolyl, furyl, thienyl, isoxazolyl, thiazolyl, furyl, phienyl, isoxazolyl, indolyl, oxetanyl, azepinyl, piperazinyl, morpholinyl, dioxanyl, thietanyl and oxazolyl. A heterocyclic aromatic group can be unsubstituted or substituted. Preferably, a heterocyclic aromatic is a monocyclic ring, wherein the ring comprises 2 to 5 carbon atoms and 1 to 3 heteroatoms.

The term “aryloxy” includes —O-aryl group, wherein aryl is as defined herein. An aryloxy group can be unsubstituted or substituted.

The term “cycloalkyl” includes a monocyclic or polycyclic saturated ring comprising carbon and hydrogen atoms and having no carbon-carbon multiple bonds. Examples of cycloalkyl groups include, but are not limited to, (C₃-C₇)cycloalkyl groups, such as cyclopropyl, cyclobutyl, cyclopentyl, cyclohexyl, and cycloheptyl, and saturated cyclic and bicyclic terpenes. A cycloalkyl group can be unsubstituted or substituted. Preferably, the cycloalkyl group is a monocyclic ring or bicyclic ring.

The terms “effective amount” or “therapeutically effective amount” refer to a sufficient amount of the agent to provide the desired biological result. That result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in a disease. An appropriate “effective” amount in any individual case may be determined by one of ordinary skill in the art using routine experimentation.

The term “halogen” includes fluorine, chlorine, bromine, and iodine.

The term “modulate” means to interact with a target either directly or indirectly so as to alter the activity of the target, including, by way of example only, to enhance the activity of the target, to inhibit the activity of the target, to limit the activity of the target, or to extend the activity of the target.

The term “modulator” means a molecule that interacts with a target either directly or indirectly. The interactions include, but are not limited to, agonist, antagonist, and the like.

By “pharmaceutically acceptable” or “pharmacologically acceptable” is meant a material which is not biologically or otherwise undesirable, i.e., the material may be administered to an individual without causing undesirable biological effects or interacting in a deleterious manner with any of the components of the composition in which it is contained.

The term “pharmaceutically acceptable salt” of a compound means a salt that is pharmaceutically acceptable and that possesses the desired pharmacological activity of the parent compound. Such salts, for example, include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; (2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine, tromethamine, N-methylglucamine, and the like. Acceptable inorganic bases include aluminum hydroxide, calcium hydroxide, potassium hydroxide, sodium carbonate, sodium hydroxide, and the like. It should be understood that a reference to a pharmaceutically acceptable salt includes the solvent addition forms or crystal forms thereof, particularly solvates or polymorphs. Solvates contain either stoichiometric or non-stoichiometric amounts of a solvent, and may be formed during the process of crystallization. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

A “prodrug” refers to a drug or compound in which the pharmacological action results from conversion by metabolic processes within the body. Prodrugs are generally drug precursors that, following administration to a subject and subsequent absorption, are converted to an active, or a more active species via some process, such as conversion by a metabolic pathway. Some prodrugs have a chemical group present on the prodrug that renders it less active and/or confers solubility or some other property to the drug. Once the chemical group has been cleaved and/or modified from the prodrug the active drug is generated. Prodrugs may be designed as reversible drug derivatives, for use as modifiers to enhance drug transport to site-specific tissues. The design of prodrugs to date has been to increase the effective water solubility of the therapeutic compound for targeting to regions where water is the principal solvent. See, e.g., Fedorak et al., Am. J. Physiol., 269:G210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987. Prodrug forms of the herein described compounds, wherein the prodrug is metabolized in vivo to produce a derivative as set forth herein are included within the scope of the claims. Indeed, some of the herein-described derivatives may be a prodrug for another derivative or active compound. The optical isomers of the compounds disclosed herein, especially those resulting from the chiral carbon atoms in the molecule. In additional embodiments of the compounds and methods provided herein, mixtures of enantiomers and/or diastereoisomers, resulting from a single preparative step, combination, or interconversion may also be useful for the applications described herein.

The term “subject” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The term “sulfonyl” refers to the presence of a sulfur atom, which is optionally linked to another moiety such as an aliphatic group, an aromatic group, an aryl group, an alicyclic group, or a heterocyclic group. Aryl or alkyl sulfonyl moieties have the formula —SO₂R′, and alkoxy moieties have the formula —O—R′, wherein R′ is alkyl, as defined herein, or is aryl wherein aryl is phenyl, optionally substituted with 1-3 substituents independently selected from halo (fluoro, chloro, bromo or iodo), lower alkyl (1-6C) and lower alkoxy (1-6C).

The terms “treat” or “treatment” are synonymous with the term “prevent” and are meant to indicate a postponement of development of diseases, preventing the development of diseases, and/or reducing severity of such symptoms that will or are expected to develop. Thus, these terms include ameliorating existing disease symptoms, preventing additional symptoms, ameliorating or preventing the underlying metabolic causes of symptoms, inhibiting the disorder or disease, e.g., arresting the development of the disorder or disease, relieving the disorder or disease, causing regression of the disorder or disease, relieving a condition caused by the disease or disorder, or stopping the symptoms of the disease or disorder.

Unless otherwise indicated, when a substituent is deemed to be “optionally substituted,” it is meant that the substituent is a group that may be substituted with one or more group(s) individually and independently selected from, for example, alkyl, cycloalkyl, aryl, heteroaryl, heteroalicyclic, hydroxy, alkoxy, aryloxy, mercapto, alkylthio, arylthio, cyano, halo, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, perhaloalkyl, perfluoroalkyl, silyl, trihalomethanesulfonyl, and amino, including mono- and di-substituted amino groups, and the protected derivatives thereof. The protecting groups that may form the protective derivatives of the above substituents are known to those of skill in the art.

The compounds described herein may be labeled isotopically (e.g. with a radioisotope) or by another other means, including, but not limited to, the use of chromophores or fluorescent moieties, bioluminescent labels, or chemiluminescent labels.

Molecular embodiments provided herein may possess one or more chiral centers and each center may exist in the R or S configuration. The compositions and methods provided herein include all diastereomeric, enantiomeric, and epimeric forms as well as the appropriate mixtures thereof. Stereoisomers may be obtained, if desired, by methods known in the art as, for example, the separation of stereoisomers by chiral chromatographic columns. Additionally, the compounds and methods provided herein may exist as geometric isomers. The compounds and methods provided herein include all cis, trans, syn, anti, entgegen (E), and zusammen (Z) isomers as well as the appropriate mixtures thereof. In some situations, compounds may exist as tautomers. All tautomers are included within the formulas described herein are provided by compounds and methods herein.

In addition, the compounds provided herein can exist in unsolvated as well as solvated forms with pharmaceutically acceptable solvents such as water, ethanol, and the like. In general, the solvated forms are considered equivalent to the unsolvated forms for the purposes of the compounds and methods provided herein.

These and other aspects of the present invention will become evident upon reference to the following detailed description. In addition, various references are set forth herein which describe in more detail certain procedures or compositions, and are incorporated by reference in their entirety.

### DISCLOSURE OF THE INVENTION

Compounds

Compounds and methods for modulating the activity of epidermal growth factor receptor (EGFR) are discussed throughout. Salts of the compounds may be used for therapeutic and prophylactic purposes, where the salt is preferably a pharmaceutically acceptable salt. Examples of pharmaceutically acceptable salts include those derived from mineral acids, such as hydrochloric, hydrobromic, phosphoric, metaphosphoric, nitric and sulphuric acids, and organic acids, such as tartaric, acetic, trifluoroacetic, citric, malic, lactic, fumaric, benzoic, glycolic, gluconic, succinic and methanesulphonic and arylsulphonic, for example Q-toluenesulphonic, acids. In another aspect, compositions containing the herein-described analogs and derivatives are provided. Preferably, the compositions are formulated to be suitable for pharmaceutical or clinical use by the inclusion of appropriate carriers or excipients. In yet another embodiment, pharmaceutical formulations are provided comprising at least one compound described herein, or a pharmaceutically acceptable salt or solvate thereof, together with one or more pharmaceutically acceptable carriers, diluents or excipients are described herein.

Synthesis of Compounds

The compounds described herein can be obtained from commercial sources, such as Aldrich Chemical Co. (Milwaukee, Wis.), Sigma Chemical Co. (St. Louis, Mo.), or Maybridge (Cornwall, England), or the compounds can be synthesized. The compounds described herein, and other related compounds having different substituents can be synthesized using techniques and materials known to those of skill in the art, such as described, for example, in March, ADVANCED ORGANIC CHEMISTRY 4^{th} Ed., (Wiley 1992); Carey and Sundberg, ADVANCED ORGANIC CHEMISTRY 3^{rd} Ed., Vols. A and B (Plenum 1992), and Green and Wuts, PROTECTIVE GROUPS IN ORGANIC SYNTHESIS 3^{rd} Ed., (Wiley 1999) (all of which are incorporated by reference in their entirety). General methods for the preparation of compound as disclosed herein may be derived from known reactions in the field, and the reactions may be modified by the use of appropriate reagents and conditions, as would be recognized by the skilled person, for the introduction of the various moieties found in the formulae as provided herein. As a guide the following synthetic methods may be utilized.

Selected examples of covalent linkages and precursor functional groups which yield them are given in the Table entitled “Examples of Covalent Linkages and Precursors Thereof.” Precursor functional groups are shown as electrophilic groups and nucleophilic groups. The functional group on the organic substance may be attached directly, or attached via any useful spacer or linker as defined below.

TABLE 1
Examples of Covalent Linkages and Precursors Thereof

| Covalent Linkage Product | Electrophile | Nucleophile |
|---|---|---|
| Carboxamides | Activated esters | amines/anilines |
| Carboxamides | acyl azides | amines/anilines |
| Carboxamides | acyl halides | amines/anilines |
| Esters | acyl halides | alcohols/phenols |
| Esters | acyl nitriles | alcohols/phenols |
| Carboxamides | acyl nitriles | amines/anilines |
| Imines | Aldehydes | amines/anilines |
| Hydrazones | aldehydes or ketones | Hydrazines |
| Oximes | aldehydes or ketones | Hydroxylamines |
| Alkyl amines | alkyl halides | amines/anilines |
| Esters | alkyl halides | carboxylic acids |
| Thioethers | alkyl halides | Thiols |
| Ethers | alkyl halides | alcohols/phenols |
| Thioethers | alkyl sulfonates | Thiols |
| Esters | alkyl sulfonates | carboxylic acids |
| Ethers | alkyl sulfonates | alcohols/phenols |
| Esters | Anhydrides | alcohols/phenols |
| Carboxamides | Anhydrides | amines/anilines |
| Thiophenols | aryl halides | Thiols |
| Aryl amines | aryl halides | Amines |
| Thioethers | Azindines | Thiols |
| Boronate esters | Boronates | Glycols |
| Carboxamides | carboxylic acids | amines/anilines |
| Esters | carboxylic acids | Alcohols |
| hydrazines | Hydrazides | carboxylic acids |
| N-acylureas or Anhydrides | carbodiimides | carboxylic acids |
| Esters | diazoalkanes | carboxylic acids |
| Thioethers | Epoxides | Thiols |
| Thioethers | haloacetamides | Thiols |
| Ammotriazines | halotriazines | amines/anilines |
| Triazinyl ethers | halotriazines | alcohols/phenols |
| Amidines | imido esters | amines/anilines |
| Ureas | Isocyanates | amines/anilines |
| Urethanes | Isocyanates | alcohols/phenols |
| Thioureas | isothiocyanates | amines/anilines |
| Thioethers | Maleimides | Thiols |
| Phosphite esters | phosphoramidites | Alcohols |
| Silyl ethers | silyl halides | Alcohols |
| Alkyl amines | sulfonate esters | amines/anilines |
| Thioethers | sulfonate esters | Thiols |
| Esters | sulfonate esters | carboxylic acids |
| Ethers | sulfonate esters | Alcohols |
| Sulfonamides | sulfonyl halides | amines/anilines |
| Sulfonate esters | sulfonyl halides | phenols/alcohols |

In general, carbon electrophiles are susceptible to attack by complementary nucleophiles, including carbon nucleophiles, wherein an attacking nucleophile brings an electron pair to the carbon electrophile in order to form a new bond between the nucleophile and the carbon electrophile.

Suitable carbon nucleophiles include, but are not limited to alkyl, alkenyl, aryl and alkynyl Grignard, organolithium, organozinc, alkyl-, alkenyl, aryl- and alkynyl-tin reagents (organostannanes), alkyl-, alkenyl-, aryl- and alkynyl-borane reagents (organoboranes and organoboronates); these carbon nucleophiles have the advantage of being kinetically stable in water or polar organic solvents. Other carbon nucleophiles include phosphorus ylids, enol and enolate reagents; these carbon nucleophiles have the advantage of being relatively easy to generate from precursors well known to those skilled in the art of synthetic organic chemistry. Carbon nucleophiles, when used in conjunction with carbon electrophiles, engender new carbon-carbon bonds between the carbon nucleophile and carbon electrophile.

Non-carbon nucleophiles suitable for coupling to carbon electrophiles include but are not limited to primary and secondary amines, thiols, thiolates, and thioethers, alcohols, alkoxides, azides, semicarbazides, and the like. These non-carbon nucleophiles, when used in conjunction with carbon electrophiles, typically generate heteroatom linkages (C—X—C), wherein X is a hetereoatom, e.g, oxygen or nitrogen.

The term “protecting group” refers to chemical moieties that block some or all reactive moieties and prevent such groups from participating in chemical reactions until the protective group is removed. It is preferred that each protective group be removable by a different means. Protective groups that are cleaved under totally disparate reaction conditions fulfill the requirement of differential removal. Protective groups can be removed by acid, base, and hydrogenolysis. Groups such as trityl, dimethoxytrityl, acetal and t-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, without limitation, methyl, ethyl, and acetyl in the presence of amines blocked with acid labile groups such as t-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be protected by conversion to simple ester derivatives as exemplified herein, or they may be blocked with oxidatively-removable protective groups such as 2,4-dimethoxybenzyl, while co-existing amino groups may be blocked with fluoride labile silyl carbamates.

Allyl blocking groups are useful in then presence of acid- and base-protecting groups since the former are stable and can be subsequently removed by metal or pi-acid catalysts. For example, an allyl-blocked carboxylic acid can be deprotected with a Pd₀-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

Typically blocking/protecting groups may be selected from:

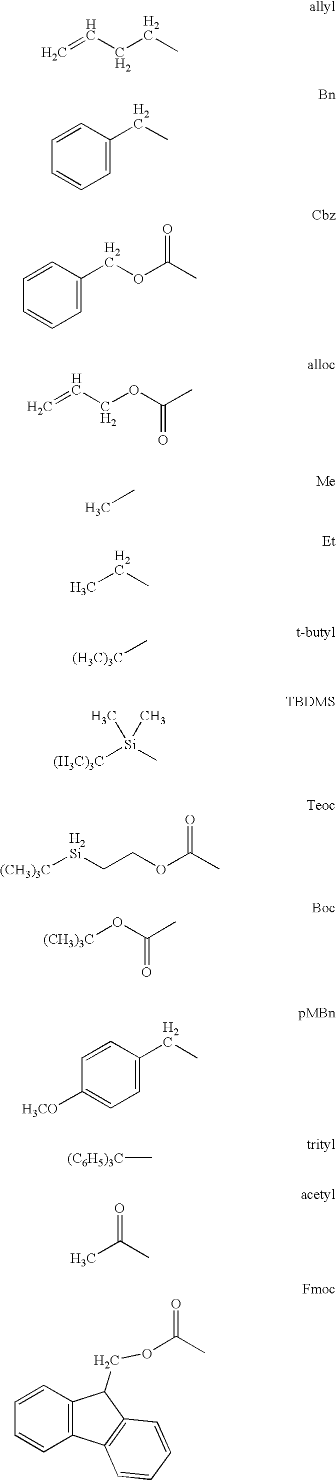

Other protecting groups are described in Greene and Wuts, Protective Groups in Organic Synthesis, 3rd Ed., John Wiley & Sons, New York, N.Y., 1999, which is incorporated herein by reference in its entirety.

Methods of Formulation and Therapeutic/Prophylactic Administation and Dosing

In practicing the methods of treatment or use provided herein, the therapeutically effective amount of the compound provided herein is administered in a pharmaceutical composition to a mammal having a condition to be treated. Preferably, the mammal is a human. The compounds described herein are preferably used to prepare a medicament, such as by formulation into pharmaceutical compositions for administration to a subject using techniques generally known in the art. A summary of such pharmaceutical and veterinary compositions as well as further information on various pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Additionally, the compounds can be used singly or as components of mixtures. In some embodiments, the compounds are those for systemic administration as well as those for topical or transdermal administration. In other embodiments, the formulations are designed for timed release. In still other embodiments, the formulation is in unit dosage form.

The composition may, for example, be in a form suitable for oral administration as a tablet, capsule, pill, powder, sustained release formulation, solution, or suspension; for parenteral injection as a sterile solution, suspension or emulsion; for topical administration as an ointment or cream; or for rectal administration as a suppository, enema, foam, or gel. The pharmaceutical composition may be in unit dosage forms suitable for single administration of precise dosages. The pharmaceutical compositions will include a conventional pharmaceutically acceptable carrier or excipient and a compound described herein as an active ingredient. In addition, it may include other medicinal or pharmaceutical agents, carriers, adjuvants, etc.

Pharmaceutical compositions described herein may contain 0.1%-95% of the compound. In any event, the composition or formulation to be administered will contain a quantity of a compound in an amount effective to alleviate or reduce the signs in the subject being treated, i.e., proliferative diseases, over the course of the treatment.

In unit dosage form, the formulation is divided into unit doses containing appropriate quantities of one or more compound. The unit dosage may be in the form of a package containing discrete quantities of the formulation. Non-limiting examples are packeted tablets or capsules, and powders in vials or ampoules.

Methods for the preparation of compositions comprising the compounds described herein include formulating the derivatives with one or more inert, pharmaceutically acceptable carriers to form either a solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. The compositions may be in liquid solutions or suspensions, solid forms suitable for solution or suspension in a liquid prior to use, or as emulsions. Suitable excipients or carriers are, for example, water, saline, dextrose, glycerol, alcohols, aloe vera gel, allantoin, glycerin, vitamin A and E oils, mineral oil, propylene glycol, PPG-2 myristyl propionate, and the like. These compositions may also contain minor amounts of nontoxic, auxiliary substances, such as wetting or emulsifying agents, pH buffering agents, and so forth.

A carrier can be one or more substances which also serve to act as a diluent, flavoring agent, solubilizer, lubricant, suspending agent, binder, or tablet disintegrating agent. A carrier can also be an encapsulating material.

In powder forms, the carrier is preferably a finely divided solid in powder form that is interdispersed as a mixture with a finely divided powder from of one or more compound. In tablet forms of the compositions, one or more compounds is intermixed with a carrier with appropriate binding properties in suitable proportions followed by compaction into the shape and size desired. Powder and tablet form compositions preferably contain between about 5 to about 70% by weight of one or more compound. Carriers that may be used in the practice include, but are not limited to, magnesium carbonate, magnesium stearate, talc, lactose, sugar, pectin, dextrin, starch, tragacanth, methyl cellulose, sodium carboxymethyl cellulose, a low-melting wax, cocoa butter, and the like.

Carriers also include any commonly used excipients in pharmaceutics and should be selected on the basis of compatibility with the compounds disclosed herein and the release profile properties of the desired dosage form. Exemplary carriers include, e.g., binders, suspending agents, disintegration agents, filling agents, surfactants, solubilizers, stabilizers, lubricants, wetting agents, diluents, and the like. Pharmaceutically acceptable carriers may comprise, e.g., acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, and the like.

The compounds described herein may also be encapsulated or microencapsulated by an encapsulating material, which may thus serve as a carrier, to provide a capsule in which the derivatives, with or without other carriers, is surrounded by the encapsulating material. In an analogous manner, cachets comprising one or more compounds are also provided. Tablet, powder, capsule, and cachet forms of the may be formulated as single or unit dosage forms suitable for administration, optionally conducted orally. For intravenous injections, the compounds described herein may be formulated in aqueous solutions, preferably in physiologically compatible buffers such as Hank's solution, Ringer's solution, or physiological saline buffer.

In suppository forms of the compositions, a low-melting wax such as, but not limited to, a mixture of fatty acid glycerides, optionally in combination with cocoa butter is first melted. One or more compounds are then dispersed into the melted material by, as a non-limiting example, stirring. The non-solid mixture is then placed into molds as desired and allowed to cool and solidify.

Non-limiting compositions in liquid form include solutions suitable for oral, injection, or parenteral administration, as well as suspensions and emulsions suitable for oral administration. Sterile aqueous based solutions of one or more compounds, optionally in the presence of an agent to increase solubility of the derivative(s), are also provided. Non-limiting examples of sterile solutions include those comprising water, ethanol, and/or propylene glycol in forms suitable for parenteral administration. A sterile solution comprising a compound described herein may be prepared by dissolving one or more compounds in a desired solvent followed by sterilization, such as by filtration through a sterilizing membrane filter as a non-limiting example. In another embodiment, one or more compounds are dissolved into a previously sterilized solvent under sterile conditions.

A water based solution suitable for oral administration can be prepared by dissolving one or more compounds in water and adding suitable flavoring agents, coloring agents, stabilizers, and thickening agents as desired. Water based suspensions for oral use can be made by dispersing one or more compounds in water together with a viscous material such as, but not limited to, natural or synthetic gums, resins, methyl cellulose, sodium carboxymethyl cellulose, and other suspending agents known to the pharmaceutical field.

The compound may be administered with the methods herein either alone or in combination with other therapies such as treatments employing other treatment agents or modalities including anti-angiogenic agents, chemotherapeutic agents, radionuclides, anti-proliferative agents, inhibitors of protein kinase C, inhibitors of other tyrosine kinases, cytokines, negative growth regulators, for example TGFβ or IFNβ, cytolytic agents, immunostimulators, cytostatic agents and the like. When co-administered with one or more biologically active agents, the compound provided herein may be administered either simultaneously with the biologically active agent(s), or sequentially. If administered sequentially, the attending physician will decide on the appropriate sequence of administering protein in combination with the biologically active agent(s).

Toxicity and therapeutic efficacy of such therapeutic regimens can be determined by standard pharmaceutical procedures in cell cultures or experimental animals, e.g. for determining the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. Compounds exhibiting high therapeutic indices are preferred. The data obtained from cell culture assays and animal studies can be used in formulating a range of dosage for use in human. The dosage of such compounds lies preferably within a range of circulating concentrations that include the ED₅₀ with minimal toxicity. The dosage may vary within this range depending upon the dosage form employed and the route of administration utilized.

The compounds can be administered before, during or after the occurrence of a condition of a disease, and the timing of administering the composition containing a compound can vary. Thus, for example, the compounds can be used as a prophylactic and can be administered continuously to subjects with a propensity to conditions and diseases in order to prevent the occurrence of the disorder. The compounds and compositions can be administered to a subject during or as soon as possible after the onset of the symptoms. The administration of the compounds can be initiated within the first 48 hours of the onset of the symptoms, preferably within the first 48 hours of the onset of the symptoms, more preferably within the first 6 hours of the onset of the symptoms, and most preferably within 3 hours of the onset of the symptoms. The initial administration can be via any route practical, such as, for example, an intravenous injection, a bolus injection, infusion over 5 minutes to about 5 hours, a pill, a capsule, transdermal patch, buccal delivery, and the like, or combination thereof. A compound is preferably administered as soon as is practicable after the onset of a condition of a condition or a disease is detected or suspected, and for a length of time necessary for the treatment of the disease, such as, for example, from about 1 month to about 3 months. The length of treatment can vary for each subject, and the length can be determined using the known criteria. For example, the compound or a formulation containing the compound can be administered for at least 2 weeks, preferably about 1 month to about 5 years, and more preferably from about 1 month to about 3 years.

The dosage appropriate for the compounds described here will be in the range of less than 0.1 mg/kg to over 10 mg/kg per day. The dosage may be a single dose or repetitive. In other embodiments using the compounds for therapeutic use, the compounds described herein are administered to a subject at dosage levels of from about 0.5 mg/kg to about 8.0 mg/kg of body weight per day. For a human subject of approximately 70 kg, this is a dosage of from 40 mg to 600 mg per day. Such dosages, however, may be altered depending on a number of variables, not limited to the activity of the compound used, the condition to be treated, the mode of administration, the requirements of the individual subject, the severity of the condition being treated, and the judgment of the practitioner.

The foregoing ranges are merely suggestive, as the number of variables in regard to an individual treatment regime is large, and considerable excursions from these recommended values are not uncommon.

Methods of Use: Biological Activity

Protein kinases (PKs) play a role in signal transduction pathways regulating a number of cellular functions, such as cell growth, differentiation, and cell death. PKs are enzymes that catalyze the phosphorylation of hydroxy groups on tyrosine, serine and threonine residues of proteins. Abnormal PK activity has been related to disorders ranging from relatively non life threatening diseases such as psoriasis to extremely virulent diseases such as glioblastoma (brain cancer). In addition, a variety of tumor types have dysfunctional growth factor receptor tyrosine kinases, resulting in inappropriate mitogenic signaling. Protein kinases are believed to be involved in many different cellular signal transduction pathways. In particular, protein tyrosine kinases (PTK) are attractive targets in the search for therapeutic agents, not only for cancer, but also against many other diseases. Blocking or regulating the kinase phosphorylation process in a signaling cascade may help treat conditions such as cancer or inflammatory processes.

Protein tyrosine kinases are a family of tightly regulated enzymes, and the aberrant activation of various members of the family is one of the hallmarks of cancer. The protein-tyrosine kinase family includes Bcr-Abl tyrosine kinase, and can be divided into subgroups that have similar structural organization and sequence similarity within the kinase domain. The members of the type III group of receptor tyrosine kinases include the platelet-derived growth factor (PDGF) receptors (PDGF receptors α and β), colony-stimulating factor (CSF-1) receptor (CSF-1R, c-Fms), FLT3, and stem cell or steel factor receptor (c-kit).

The compounds, compositions, and methods provided herein are useful to modulate the activity of kinases including, but not limited to, ERBB2, ABL, AURKA, CDK2, EGFR, FGFR1, LCK, MAPK14, PDGFR, KDR, ABL, BRAF, ERBB4, FLT3, KIT, and RAF1. In some embodiments, the compositions and methods provided herein modulate the activity of a mutant kinase.

Inhibition by the compounds provided herein can be determined using any suitable assay. In one embodiment, inhibition is determined in vitro. In a specific embodiment, inhibition is assessed by phosphorylation assays. Any suitable phosphorylation assay can be employed. For example, membrane autophosphorylation assays, receptor autophosphorylation assays in intact cells, and ELISA's can be employed. See, e.g., Gazit, et al., J. Med. Chem. (1996) 39:2170-2177, Chapter 18 in CURRENT PROTOCOLS IN MOLECULAR BIOLOGY (Ausubel, et al., eds. 2001). Cells useful in such assays include cells with wildtype or mutated forms. In one embodiment, the wildtype is a kinase that is not constitutively active, but is activated with upon dimerization. For example, the mutant FLT3 kinase is constitutively active via internal tandem duplication mutations or point mutations in the activation domain. Suitable cells include those derived through cell culture from patient samples as well as cells derived using routine molecular biology techniques, e.g., retroviral transduction, transfection, mutagenesis, etc. Exemplary cells include Ba/F3 or 32Dc13 cells transduced with, e.g., MSCV retroviral constructs FLT3-ITD (Kelly et al., 2002); Molm-13 and Molm14 cell line (Fujisaki Cell Center, Okayama, Japan); HL60 (AML-M3), AML193 (AML-M5), KG-1, KG-1a, CRL-1873, CRL-9591, and THP-1 (American Tissue Culture Collection, Bethesda, Md.); or any suitable cell line derived from a patient with a hematopoietic malignancy.

In some embodiments, the compounds described herein significantly inhibit receptor tyrosine kinases. A significant inhibition of a receptor tyrosine kinase activity refers to an IC₅₀ of less than or equal to 100 μM. Preferably, the compound can inhibit activity with an IC₅₀ of less than or equal to 50 μM, more preferably less than or equal to 10 μM, more preferably less than 1 μM, or less than 100 nM, most preferably less than 50 nM. Lower IC₅₀'s are preferred because the IC₅₀ provides an indication as to the in vivo effectiveness of the compound. Other factors known in the art, such as compound half-life, biodistribution, and toxicity should also be considered for therapeutic uses. Such factors may enable a compound with a lower IC₅₀ to have greater in vivo efficacy than a compound having a higher IC₅₀ Preferably, a compound that inhibits activity is administered at a dose where the effective tyrosine phosphorylation, i.e., IC₅₀, is less than its cytotoxic effects, LD₅₀.

In some embodiments, the compounds selectively inhibit one or more kinases. Selective inhibition of EGFR is achieved by inhibiting activity of one kinase, while having an insignificant effect on other members of the superfamily.

The compounds disclosed herein are useful in treating conditions characterized by any inappropriate EGFR activity, such as particularly proliferative disorders. Such activity includes, but is not limited to enhanced or decreased EGFR activity resulting from increased or de novo expression of EGFR in cells, increased EGFR-ligand expression or activity, and EGFR mutations resulting in constitutive activation. The existence of inappropriate or abnormal EGFR-ligand and EGFR levels or activity can be determined using well known methods in the art. For example, abnormally high EGFR ligand levels can be determined using commercially available ELISA kits. EGFR levels can be determined using flow cytometric analysis, immunohistochemical analysis, in situ hybridization techniques.

The compounds, compositions, and methods described can be used to treat a variety of diseases and unwanted conditions associated EGFR activity, including, but not limited to, blood vessel growth (angiogenesis), cancer, benign hyperplasia, keloid formation, and psoriasis. In one aspect, the compounds are used to reduce the likelihood of occurrence of a cancer. In other embodiments, the compounds are used to treat non-small cell lung cancer or other solid tumors that overexpress EGF receptors. In still other embodiments, the compounds are useful for treating head cancer, neck cancer, pancreatic cancer, hepatocellular carcinoma, esophageal cancer, breast cancer, ovarian cancer, gynealogical cancer, colorectal cancer, and glioblastoma.

Compounds identified herein as inhibitors of EGFR activity can be used to prevent or treat a variety of diseases and unwanted conditions, including, but not limited to benign or malignant tumors, e.g., carcinoma of the kidneys, liver, adrenal glands, bladder, breast, stomach, ovaries, colon, rectum, prostate, pancreas, lungs, vagina or thyroid, sarcoma, glioblastomas, numerous tumors of the neck and head, and leukemia. In one embodiment, the malignancy is of epithelial origin. In another embodiment, the compounds are used to treat or prevent non-small cell lung carcinoma. In still another embodiment, the disease treated by the compounds disclosed herein is pancreatic cancer. The compounds may be useful in inducing the regression of tumors as well as preventing the seeding and outgrowth of tumor metastases. These compounds are also useful in therapeutically or prophylactically in diseases or disorders associated with non-malignant hyperplasia, e.g., epidermal hyperproliferation (e.g., psoriasis), keloid formation, prostate hyperplasia, and cardiac hypertrophy. It is also possibly to use the compounds disclosed herein in the treatment of diseases of the immune system and the central and peripheral nervous systems insofar as EGFR or EGFR-related receptors are involved.

Activity towards EGFR refers to one or more of the biologically relevant activity associated with EGFR, including but not limited to autophosphorylation, phosphorylation of other substrates, anti-apoptotic activity, proliferative activity, and differentiation activity. In this context, inhibition and reduction of the activity of EGFR refers to a lower level of measured activity relative to a control experiment in which the protein, cell, or subject is not treated with the test compound or is treated with a compound that does not inhibit EGFR activity, whereas an increase in the activity of EGFR refers to a higher level of measured activity relative to a control experiment. In particular embodiments, the reduction or increase is at least 10%. Reduction or increase in the activity of EGFR of at least 20%, 50%, 75%, 90% or 100% or any integer between 10% and 100%, may be preferred for particular applications. The compounds disclosed herein modulate at least one of the activities mediated by EGFR, e.g. anti-apoptotic activity, and can modulate one or more or all of the known EGFR activities.

Aberrant or inappropriate EGFR activity can be determined by an increase in one or more of the activities occurring subsequent to binding of a ligand, e.g., EGF, TGFα, amphiregulin, HB-EGF, betacellulin, epiregulin, or epigen: 1) phosphorylation or autophosphorylation of EGFR; 2) phosphorylation of a EGFR substrate, e.g., Stat5b, phospholipase gamma (PLCγ); 3) activation of a related complex, e.g. PI3K; 4) activation of other genes, e.g., c-fos; and 5) cellular proliferation. These activities are readily measured by well known methods in the art. For example, tyrosine phosphorylation can be determined using e.g., immunoblotting with anti-phosphotyrosine antibodies. See, e.g., Chapter 18 in CURRENT PROTOCOLS IN MOLECULAR BIOLOGY (Ausubel, et al., eds. 2001). Cell proliferation can be determined using, e.g., ³H-thymidine uptake.

Compounds described herein are contacted with EGFR expressing cells in any suitable manner. The cell may constitutively or inducibly express EGFR following exogenous or endogenous stimuli or recombinant manipulation. The cell can be in vitro or in vivo in a tissue or organ. The cell and the compounds disclosed herein can be contacted for any period of time where undesirable toxicity results. Contacting an EGFR-expressing cell in vivo includes systemic, localized, and targeted delivery mechanisms known in the art. See e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

The action of the compounds disclosed herein on the EGFR ligand-stimulated cellular tyrosine phosphorylation of EGFR can be also determined in the human A431. In one embodiment, the compounds disclosed exhibit inhibition at concentrations in the nanomolar to micromolar range. Additionally, inhibition can be determined by examining gene expression profiles of EGFR-ligand treated cells. For example, the stimulation of dormant BALB-c3T3 cell by EGF rapidly induces the expression of c-fos mRNA. Pretreatment of the cells with a compound disclosed herein prior to the stimulation with EGF can inhibit the c-fos expression. See Trinks et al., J. Med. Chem. 37(7), 1015-27 (1994).

EGFR inhibition by the compounds provided herein can be determined using any suitable assay. In one embodiment, EGFR inhibition is determined in vitro. In a specific embodiment, EGFR inhibition is assessed by phosphorylation assays. Any suitable phosphorylation assay can be employed. For example, membrane autophosphorylation assays, receptor autophosphorylation assays in intact cells, and ELISA's can be employed. See, e.g., McGlynn et al., Eur. J. Biochem. 207:265-75(1992); Trinks et al., J. Med. Chem. 37(7), 1015-27(1994); Posner et al., J. Biol. Chem. 267(29):20638-47 (1992); Chapter 18 in CURRENT PROTOCOLS IN MOLECULAR BIOLOGY (Ausubel, et al., eds. 2001). Cells useful in such assays include, but are not limited to MDA-MB-231, Hs578T, A431, MCF-7, T-47D, ZA-75-1, SUM44, epidermoid Balb/c mouse keratinocyte cells, and cells recombinantly engineered to express EGFR, including NIH-3T3, CHO and COS cells (American Type Culture Collection, Rockville, Md.). See e.g., Roos et al., Proc. Natl. Acad. Sci. U.S.A. 83:991-95 (1986).

In some embodiments, the compounds selectively inhibit one or more kinases. For example, selective inhibition of EGFR is achieved by significantly inhibiting EGFR activity, while having an insignificant effect (i.e., an IC₅₀ for tyrosine phosphorylation greater than 100 μM on PDGFR) on other members of the PDGFR superfamily. The compounds described can inhibit the activation of the EGFR by one or more of the ligands or EGFR receptors, i.e., erbB2, erbB3, or erbB4. Members of the PDGFR superfamily, besides PDGFR, include EGFR. KDR, and Flt1. In some embodiments, no other member of the PDGFR super family, is significantly inhibited. In one embodiment, compounds inhibit EGFR significantly more than erbB2, erbB3, or erbB4.

In addition to or instead of inhibiting the EGFR tyrosine kinase, the compounds disclosed herein can, in one embodiment, also inhibit other tyrosine protein kinases that are involved in the signal transmission mediated by other trophic factors which function in growth regulation and transformation in mammal cells, including human cells. Exemplary kinases include, but are limited to the ab1 kinase, e.g., the v-ab1 kinase (Lydon et al., Oncogene Res. 5:161-73 (1990) and Geissler et al., Cancer Res. 52:4492-98 (1992)); kinases of the src kinase family, e.g., the c-src kinase, lck kinase and fyn kinase; other members of the PDGFR tyrosine kinase family, e.g., PDGFR, CSF-1R, Kit, VEGFR and FGFR; and the insulin-like growth factor receptor kinase (IGF-1-kinase), and serine/threonine kinases, e.g., protein kinase C.

In one embodiment, the efficacy of the EGFR modulation is determined using cellular proliferation assays. Briefly, cells expressing EGFR are co-cultured in the presence of the inhibitor and EGF, TGF-α, or other appropriate EGFR ligand. See, e.g., Weissmann et al., Cell 32, 599 (1983) and Carpenter et al., Anal. Biochem. 153:279-82 (1985). The compound is inhibitory for proliferation if it inhibits the proliferation of cells relative to the proliferation of cells in the absence of the compound or in the presence of a non-EGFR inhibitor. Proliferation may be quantified using any suitable methods. Typically, the proliferation is determined by assessing the incorporation of radioactive-labeled nucleotides into DNA (e.g., ³H-thymidine) in vitro. In one embodiment, proliferation is determined by ATP luminescence, e.g., CellTiter-Glo™ Luminescent Cell Viability Assay (Promega). In another embodiment, inhibition of EFGR by the compounds presented herein is determined by cell cycle analysis. See generally CYTOKINE CELL BIOLOGY: A PRACTICAL APPROACH (F. Balkwell, ed. 2000). Analogous methods may be used with the other protein kinases described herein, including by way of example only, FLT3, PDGFR, and Bcr-Abl.

In one embodiment, the compounds disclosed herein can be used to treat cell proliferative disorders. Cell proliferative disorders are disorders wherein undesirable cell proliferation of one or more cellular subset in an organism occurs and results in harm, e.g., discomfort, reduction or loss of function, or decreased life expectancy, to the organism. A cellular proliferative disorder mediated by EGFR activation can be determined by examining the level of EGFR activity using the methods disclosed herein. Analogous methods may be used with the other protein kinases described herein, including by way of example only, FLT3, PDGFR, and Bcr-Abl.

In another embodiment, EGFR inhibition is determined in vivo. In one embodiment, animal models of tumor growth are used to assess the efficacy of EGFR inhibitors against tumor growth and metastasis in vivo. Any suitable animal model may be employed to assess the anti-tumor activity of EGFR inhibitors. The murine recipient of the tumor can be any suitable strain. The tumor can be syngeneic, allogeneic, or xenogeneic to the tumor. The tumor can express endogenous or exogenous EGFR. Exogenous EGFR expression can be achieved using well known methods of recombinant expression via transfection or transduction of the cells with the appropriate nucleic acid. The recipient can be immunocompetent or immunocompromised in one or more immune-related functions, included but not limited to nu/nu, SCID, and beige mice. In one specific embodiment, the mouse is a Balb/c or C57BL/6 mouse. Any suitable tumor cells from fresh tumor samples, and short term polyclonal tumor cells. Exemplary tumor cell lines include EGFR transfected NIH3T3, MCF7 (human mammary), and A431 (human epidermoid) cells. See e.g., Santon et al., Cancer Res. 46:4701-05 (1986) and Ozawa et al, Int. J. Cancer 40:706-10 (1987). The dosage of EGFR inhibitory compound ranges from 1 μg/mouse to 1 mg/mouse in at least one administration. The compound can be administered by any suitable route, including subcutaneous, intravenous, intraperitoneal, intracerebral, intradermal, or implantation of tumor fragments. In one embodiment, the dose of compound is 100 μg/mouse twice a week. In one specific embodiment, the tumor is injected subcutaneously at day 0, and the volume of the primary tumor is measured at designated time points by using calipers. Any suitable control compound can be used. Pharmacokinetics, oral bioavailability, and dose proportionality studies can be performed in these animals using well known methods. See, e.g., Klutchko, et al., J. Med. Chem. (1998) 41:3276-3292. Analogous methods may be used with the other protein kinases described herein, including by way of example only, FLT3, PDGFR, and Bcr-Abl.

Aberrant activity of protein tyrosine kinases, such as c-erbB2, c-src, c-met, EGFR and PDGFR have been implicated in human malignancies. Elevated EGFR activity has, for example, been implicated in non-small cell lung, bladder and head and neck cancers, and increased c-erbB2 activity in breast, ovarian, gastric and pancreatic cancers. Inhibition of protein tyrosine kinases should therefore provide a treatment for tumors such as those described herein.

Methods of Use

By modulating kinase activity, the compounds disclosed herein can be used to treat a variety of diseases. Suitable conditions characterized by undesirable protein-kinase activity can be treated by the compounds presented herein. As used herein, the term “condition” refers to a disease, disorder, or related symptom where inappropriate kinase activity is present. In some embodiments, these conditions are characterized by aggressive neovasculaturization including tumors, especially acute myelogenous leukemia (AML), B-precursor cell acute lymphoblastic leukemias, myelodysplastic leukemias, T-cell acute lymphoblastic leukemias, and chronic myelogenous leukemias (CMLs). In some embodiments, an EGFR-modulating compounds may be used to treat tumors.

Compounds presented herein are useful in the treatment of a variety of biologically aberrant conditions or disorders related to tyrosine kinase signal transduction. Such disorders pertain to abnormal cell proliferation, differentiation, and/or metabolism. Abnormal cell proliferation may result in a wide array of diseases, including the development of neoplasia such as carcinoma, sarcoma, leukemia, glioblastoma, hemangioma, psoriasis, arteriosclerosis, arthritis and diabetic retinopathy (or other disorders related to uncontrolled angiogenesis and/or vasculogenesis).

In various embodiments, compounds presented herein regulate, modulate, and/or inhibit disorders associated with abnormal cell proliferation by affecting the enzymatic activity of one or more tyrosine kinases and interfering with the signal transduced by said kinase. More particularly, provided herein are compounds which regulate, modulate said kinase mediated signal transduction pathways as a therapeutic approach to cure leukemia and many kinds of solid tumors, including but not limited to carcinoma, sarcoma, erythroblastoma, glioblastoma, meningioma, astrocytoma, melanoma and myoblastoma. Indications may include, but are not limited to brain cancers, bladder cancers, ovarian cancers, gastric cancers, pancreas cancers, colon cancers, blood cancers, lung cancers and bone cancers.

In other embodiments, compounds herein are useful in the treatment of cell proliferative disorders including cancers, blood vessel proliferative disorders, fibrotic disorders, and mesangial cell proliferative disorders. Blood vessel proliferation disorders refer to angiogenic and vasculogenic disorders generally resulting in abnormal proliferation of blood vessels. The formation and spreading of blood vessels, or vasculogenesis and angiogenesis, respectively, play important roles in a variety of physiological processes such as embryonic development, corpus luteum formation, wound healing and organ regeneration. They also play a pivotal role in cancer development. Other examples of blood vessel proliferation disorders include arthritis, where new capillary blood vessels invade the joint and destroy cartilage, and ocular diseases, like diabetic retinopathy, where new capillaries in the retina invade the vitreous, bleed and cause blindness. Conversely, disorders related to the shrinkage, contraction or closing of blood vessels, such as restenosis, are also implicated.

Fibrotic disorders refer to the abnormal formation of extracellular matrix. Examples of fibrotic disorders include hepatic cirrhosis and mesangial cell proliferative disorders. Hepatic cirrhosis is characterized by the increase in extracellular matrix constituents resulting in the formation of a hepatic scar. Hepatic cirrhosis can cause diseases such as cirrhosis of the liver. An increased extracellular matrix resulting in a hepatic scar can also be caused by viral infection such as hepatitis. Lipocytes appear to play a major role in hepatic cirrhosis. Other fibrotic disorders implicated include atherosclerosis.

Mesangial cell proliferative disorders refer to disorders brought about by abnormal proliferation of mesangial cells. Mesangial proliferative disorders include various human renal diseases, such as glomerulonephritis, diabetic nephropathy, malignant nephrosclerosis, thrombotic microangiopathy syndromes, transplant rejection, and glomerulopathies. The cell proliferative disorders which are indications of the compounds and methods provided herein are not necessarily independent. For example, fibrotic disorders may be related to, or overlap, with blood vessel proliferative disorders. For example, atherosclerosis results, in part, in the abnormal formation of fibrous tissue within blood vessels.

Compounds provided herein can be administered to a subject upon determination of the subject as having a disease or unwanted condition that would benefit by treatment with said derivative. The determination can be made by medical or clinical personnel as part of a diagnosis of a disease or condition in a subject. Non-limiting examples include determination of a risk of acute myelogenous leukemia (AML), B-precursor cell acute lymphoblastic leukemias, myelodysplastic leukemias, T-cell acute lymphoblastic leukemias, and chronic myelogenous leukemias (CMLs).

The methods provided herein can comprise the administration of an effective amount of one or more compounds as disclosed herein, optionally in combination with one or more other active agents for the treatment of a disease or unwanted condition as disclosed herein. The subject is preferably human, and repeated administration over time is within the scope of the methods provided herein.

Also provided herein are compounds described throughout and their salts or solvates and pharmaceutically acceptable salts or solvates thereof for use in the prevention or treatment of disorders mediated by aberrant protein tyrosine kinase activity such as human malignancies and the other disorders mentioned herein. The compounds provided herein are especially useful for the treatment of disorders caused by aberrant kinase activity such as breast, ovarian, gastric, pancreatic, non-small cell lung, bladder, head and neck cancers, and psoriasis. The cancers include hematologic cancers, for example, acute myelogenous leukemia (AML), B-precursor cell acute lymphoblastic leukemias, myelodysplastic leukemias, T-cell acute lymphoblastic leukemias, and chronic myelogenous leukemias (CMLs).

A further aspect provided herein are methods of treatment of a human or animal subject suffering from a disorder mediated by aberrant protein tyrosine kinase activity, including susceptible malignancies, which comprises administering to the subject an effective amount of a compound described herein or a pharmaceutically acceptable salt or solvate thereof.

A further aspect provided herein is the use of a compound described herein, or a pharmaceutically acceptable salt or solvate thereof, in the preparation of a medicament for the treatment of cancer and malignant tumors. The cancer can be stomach, gastric, bone, ovary, colon, lung, brain, larynx, lymphatic system, genitourinary tract, ovarian, squamous cell carcinoma, astrocytoma, Kaposi's sarcoma, glioblastoma, lung cancer, bladder cancer, head and neck cancer, melanoma, ovarian cancer, prostate cancer, breast cancer, small-cell lung cancer, leukemia, acute myelogenous leukemia (AML), B-precursor cell acute lymphoblastic leukemias, myelodysplastic leukemias, T-cell acute lymphoblastic leukemias, and chronic myelogenous leukemias (CMLs), glioma, colorectal cancer, genitourinary cancer gastrointestinal cancer, or pancreatic cancer.

Compounds provided herein are useful for preventing and treating conditions associated with ischemic cell death, such as myocardial infarction, stroke, glaucoma, and other neurodegenerative conditions. Various neurodegenerative conditions which may involve apoptotic cell death, include, but are not limited to, Alzheimer's Disease, ALS and motor neuron degeneration, Parkinson's disease, peripheral neuropathies, Down's Syndrome, age related macular degeneration (ARMD), traumatic brain injury, spinal cord injury, Huntington's Disease, spinal muscular atrophy, and HIV encephalitis. The compounds described in detail herein can be used in methods and compositions for imparting neuroprotection and for treating neurodegenerative diseases.

The compounds described herein, can be used in a pharmaceutical composition for the prevention and/or the treatment of a condition selected from the group consisting of arthritis (including osteoarthritis, degenerative joint disease, spondyloarthropathies, gouty arthritis, systemic lupus erythematosus, juvenile arthritis and rheumatoid arthritis), common cold, dysmenorrhea, menstrual cramps, inflammatory bowel disease, Crohn's disease, emphysema, acute respiratory distress syndrome, asthma, bronchitis, chronic obstructive pulmonary disease, Alzheimer's disease, organ transplant toxicity, cachexia, allergic reactions, allergic contact hypersensitivity, cancer (such as solid tumor cancer including colon cancer, breast cancer, lung cancer and prostrate cancer; hematopoietic malignancies including leukemias and lymphomas; Hodgkin's disease; aplastic anemia, skin cancer and familiar adenomatous polyposis), tissue ulceration, peptic ulcers, gastritis, regional enteritis, ulcerative colitis, diverticulitis, recurrent gastrointestinal lesion, gastrointestinal bleeding, coagulation, anemia, synovitis, gout, ankylosing spondylitis, restenosis, periodontal disease, epidermolysis bullosa, osteoporosis, atherosclerosis (including atherosclerotic plaque rupture), aortic aneurysm (including abdominal aortic aneurysm and brain aortic aneurysm), periarteritis nodosa, congestive heart failure, myocardial infarction, stroke, cerebral ischemia, head trauma, spinal cord injury, neuralgia, neurodegenerative disorders (acute and chronic), autoimmune disorders, Huntington's disease, Parkinson's disease, migraine, depression, peripheral neuropathy, pain (including low back and neck pain, headache and toothache), gingivitis, cerebral amyloid angiopathy, nootropic or cognition enhancement, amyotrophic lateral sclerosis, multiple sclerosis, ocular angiogenesis, corneal injury, macular degeneration, conjunctivitis, abnormal wound healing, muscle or joint sprains or strains, tendonitis, skin disorders (such as psoriasis, eczema, scleroderma and dermatitis), myasthenia gravis, polymyositis, myositis, bursitis, burns, diabetes (including types I and II diabetes, diabetic retinopathy, neuropathy and nephropathy), tumor invasion, tumor growth, tumor metastasis, corneal scarring, scleritis, immunodeficiency diseases (such as AIDS in humans and FLV, FIV in cats), sepsis, premature labor, hypoprothrombinemia, hemophilia, thyroiditis, sarcoidosis, Behcet's syndrome, hypersensitivity, kidney disease, Rickettsial infections (such as Lyme disease, Erlichiosis), Protozoan diseases (such as malaria, giardia, coccidia), reproductive disorders, and septic shock, arthritis, fever, common cold, pain and cancer in a mammal, preferably a human, cat, livestock or a dog, comprising an amount of a compound described herein or a pharmaceutically acceptable salt thereof effective in such prevention and/or treatment optionally with a pharmaceutically acceptable carrier.

A further aspect provided herein is the use of a compound described herein, or a pharmaceutically acceptable salt thereof, in the preparation of a medicament for the treatment of psoriasis.

Kits/Articles of Manufacture

For use in the therapeutic applications described herein, kits and articles of manufacture are also described herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers can be formed from a variety of materials such as glass or plastic.

For example, the container(s) can comprise one or more compounds described herein, optionally in a composition or in combination with another agent as disclosed herein. The container(s) optionally have a sterile access port (for example the container can be an intravenous solution bag or a vial having a stopper pierceable by a hypodermic injection needle). Such kits optionally comprising a compound with an identifying description or label or instructions relating to its use in the methods described herein.

A kit will typically may comprise one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

The terms “kit” and “article of manufacture” may be used as synonyms.

For the sake of brevity, all patents and other references cited herein are incorporated by reference in their entirety.

### EXAMPLES

The compounds and methods provided herein are further illustrated by the following examples, which should not be construed as limiting in any way. The experimental procedures to generate the data shown are discussed in more detail below. For all formulations herein, multiple doses may be proportionally compounded as is known in the art.

The compounds and methods provided herein have been described in an illustrative manner, and it is to be understood that the terminology used is intended to be in the nature of description rather than of limitation.

Compound A1

### 4-(3-Chloro-benzyloxy)-7H-pyrrolo[2,3-d]pyrimidine; LC-MS: 260 (M⁺+H)

Compound A1 may be synthesized by the following procedure:

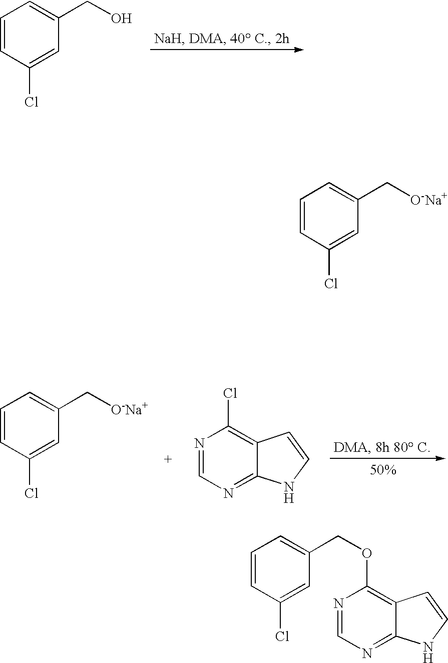

To a 1 mM solution of 3-chlorobenzyl alcohol was added 1.5 eq of NaH in DMA and stirred at 40° C. for 2 hours. The solution was cooled and 0.95 eq of 4-Chloro-7H-pyrrolo[2,3-d]pyrimidine in 0.5 ml DMA was added and the reaction heated at 80° C. for 8 hours. The reaction was cooled to room temperature and purified by reverse phase HPLC (Water:Acetonitrile solvent system) to obtain 2 mg (50%) of compound A1.

Compounds A2 through A9 were synthesized in a manner analogous to Compound A1 using similar starting materials and reagents. The structures are shown below in Table A:

TABLE A

| | CHEMICAL |
|---|---|
| NO. | STRUCTURE |
| A1 | 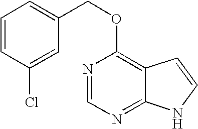 |
| A2 | 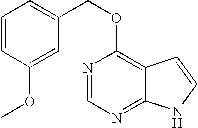 |
| A3 | 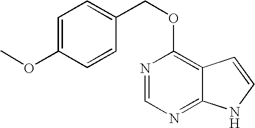 |
| A4 | 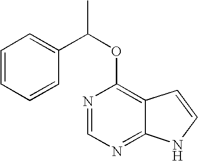 |
| A5 | 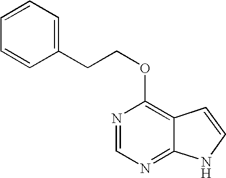 |
| A6 | 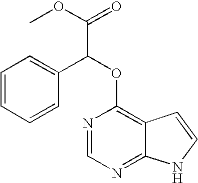 |
| A7 | 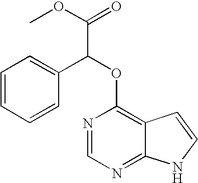 |
| A8 | 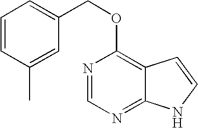 |
| A9 | 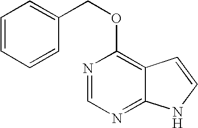 |

Compound B1

### 4-(2-Chloro-phenoxy)-7H-pyrrolo[2,3-d]pyrimidine; LC-MS: 246 (M⁺+H)

Compound B1 was synthesized by the following procedure:

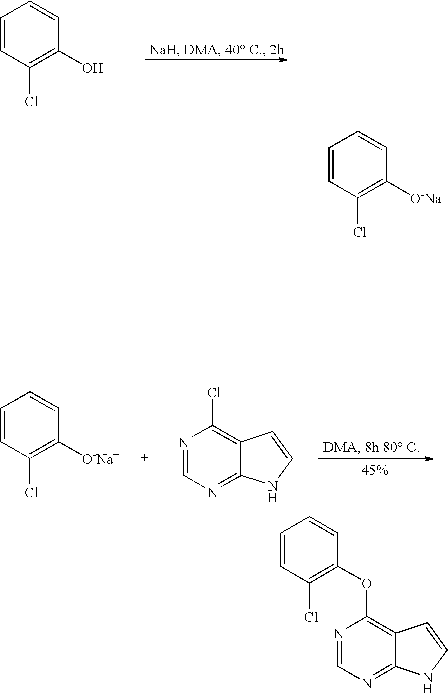

To a 1 mM solution of 2-chlorophenol was added 1.5 eq of NaH in DMA and stirred at 40° C. for 2 hours. The solution was cooled and 0.95 eq of 4-Chloro-7H-pyrrolo[2,3-d]pyrimidine in 0.5 ml DMA was added and the reaction heated at 80° C. for 8 hours. The reaction was cooled to room temperature and purified by reverse phase HPLC (Water:Acetonitrile solvent system) to obtain 2 mg (45%) of compound B1.

Compounds B2 through B22 were synthesized in a manner analogous to Compound B1 using similar starting materials and reagents. The structures are shown below in Table B:

TABLE B

| | CHEMICAL |
|---|---|
| NO. | STRUCTURE |
| B1 | 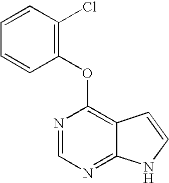 |
| B2 | 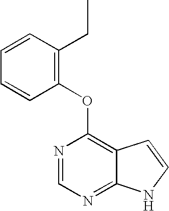 |
| B3 | 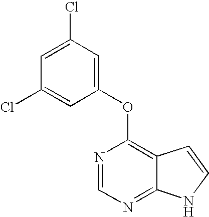 |
| B4 | 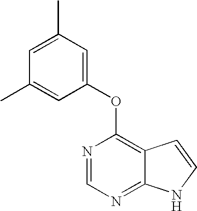 |
| B5 | 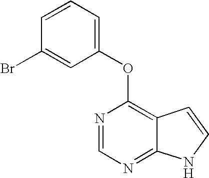 |
| B6 | 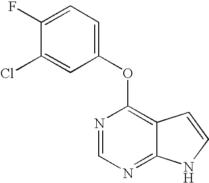 |
| B7 | 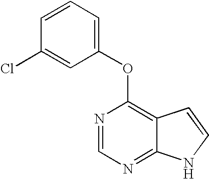 |
| B8 | 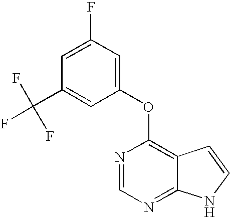 |
| B9 | 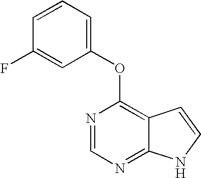 |
| B10 | 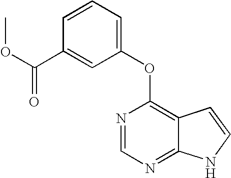 |
| B11 | 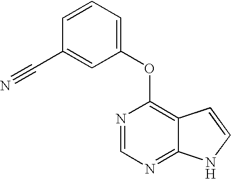 |
| B12 | 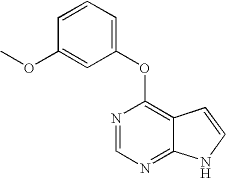 |
| B13 | 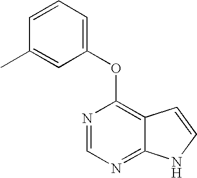 |
| B14 | 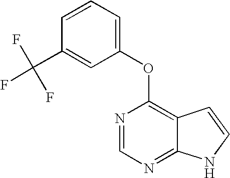 |
| B15 | 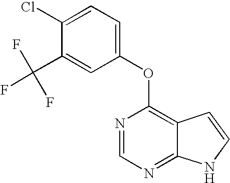 |
| B16 | 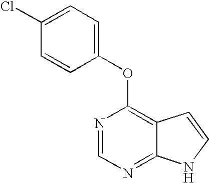 |
| B17 | 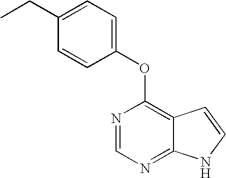 |
| B18 | 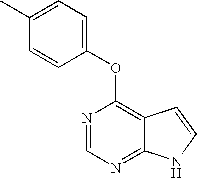 |
| B19 | 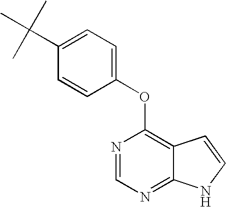 |
| B20 | 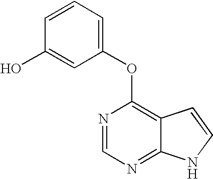 |
| B21 | 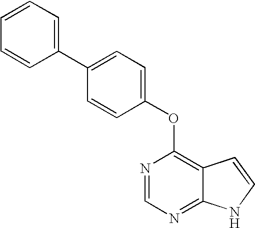 |
| B22 | 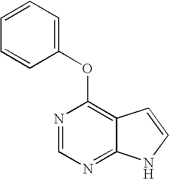 |

Compound C1

### 4-(3-Chloro-benzyloxy)-6-(4-methoxy-phenyl)-7H-pyrrolo[2,3-d]pyrimidine; LC-MS: 367 (M⁺+H)

Compound C1 was synthesized by the following procedure:

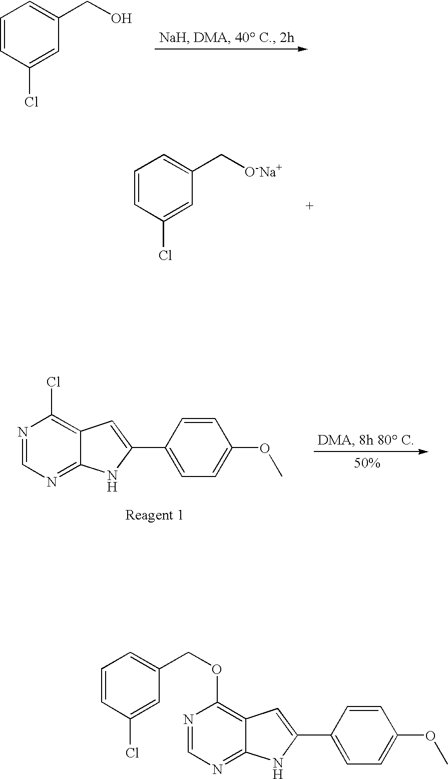

To a 1 mM solution of 3-chlorobenzyl alcohol was added 1.5 eq of NaH in DMA and stirred at 40° C. for 2 hours. The solution was cooled and 0.95 eq of reagent 1 in 0.5 ml DMA was added and the reaction heated at 80° C. for 8 hours. The reaction was cooled to room temperature and purified by reverse phase HPLC (Water:Acetonitrile solvent system) to obtain 2 mg (50%) of compound C1.

Compounds C2 through C4 were synthesized in a manner analogous to Compound C1 using similar starting materials and reagents. The structures are shown below in Table C:

TABLE C

| | CHEMICAL |
|---|---|
| NO. | STRUCTURE |
| C1 | 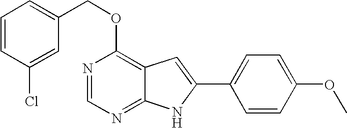 |
| C2 | 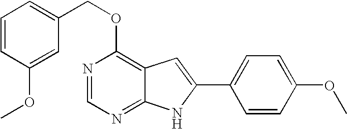 |
| C3 | 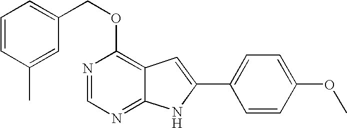 |
| C4 | 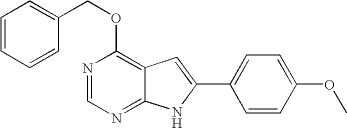 |

Compound D1

### 4-(2-Ethyl-phenoxy)-6-(4-methoxy-phenyl)-7H-pyrrolo[2,3-d]pyrimidine; LC-MS: 347 (M⁺+H)

Compound D1 was synthesized by the following procedure:

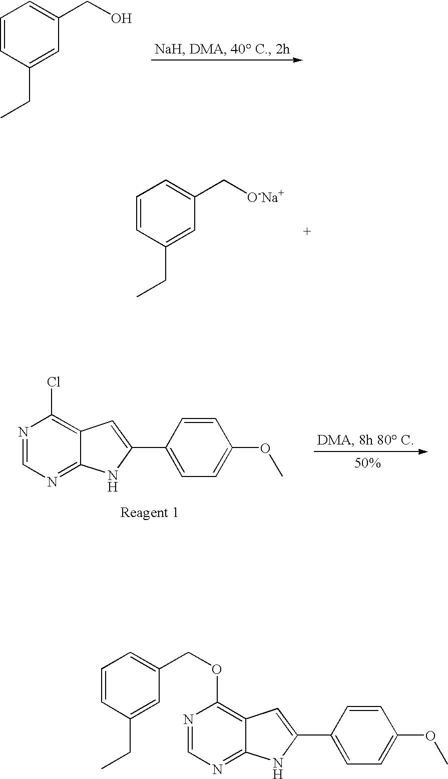

To a 1 mM solution of 2-chlorophenol was added 1.5 eq of NaH in DMA and stirred at 40° C. for 2 hours. The solution was cooled and 0.95 eq reagent 1 in 0.5 ml DMA was added and the reaction heated at 80° C. for 8 hours. The reaction was cooled to room temperature and purified by reverse phase HPLC (Water:Acetonitrile solvent system) to obtain 2 mg (45%) of compound D1.

Compounds D2 through D24 were synthesized in a manner analogous to Compound D1 using similar starting materials and reagents. The structures are shown below in Table D:

TABLE D

| | CHEMICAL |
|---|---|
| NO. | STRUCTURE |
| D1 | 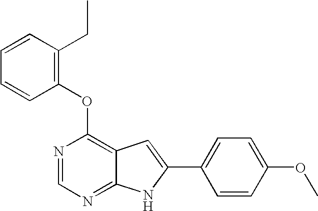 |
| D2 | 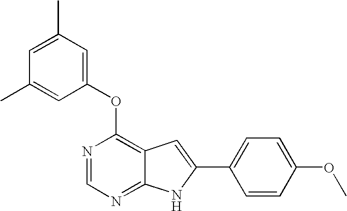 |
| D3 | 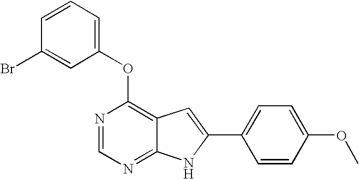 |
| D4 | 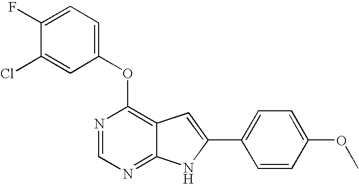 |
| D5 | 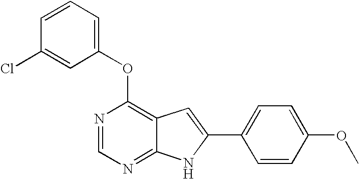 |
| D6 | 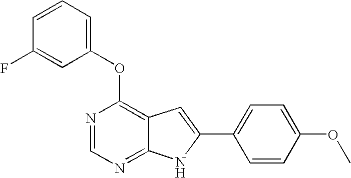 |
| D7 | 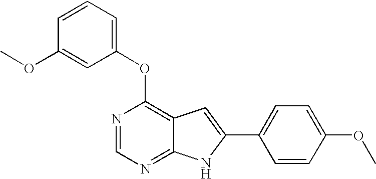 |
| D8 | 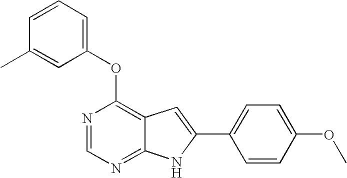 |
| D9 | 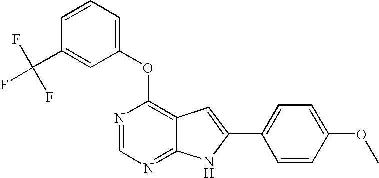 |
| D10 | 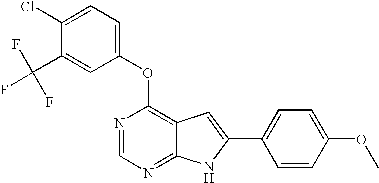 |
| D11 AB40145 | 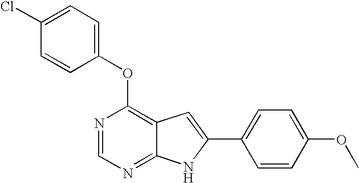 |
| D12 | 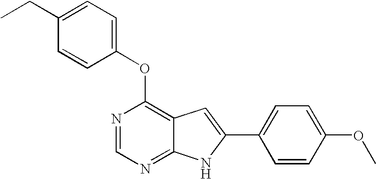 |
| D13 | 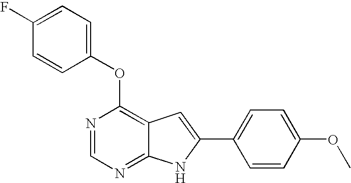 |
| D14 | 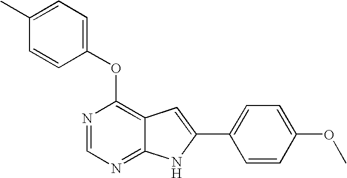 |
| D15 | 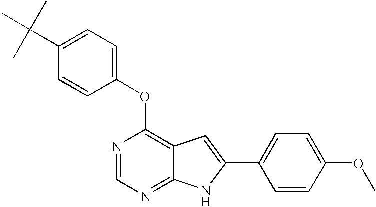 |
| D16 | 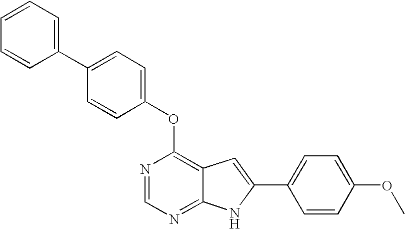 |
| D17 | 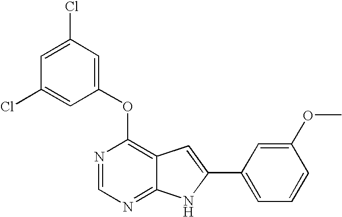 |
| D18 | 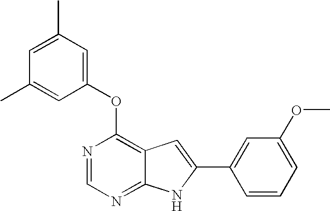 |
| D19 | 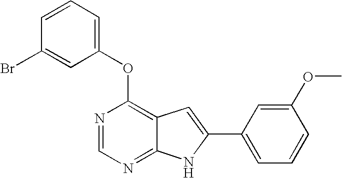 |
| D20 | 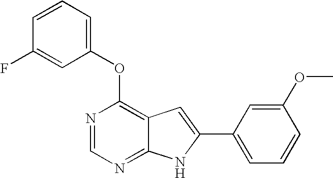 |
| D21 | 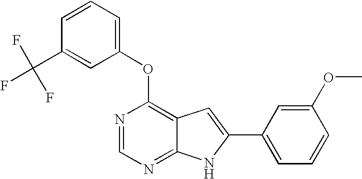 |
| D22 | 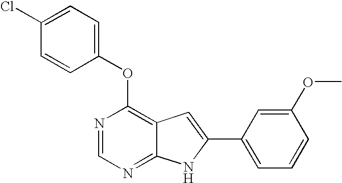 |
| D23 | 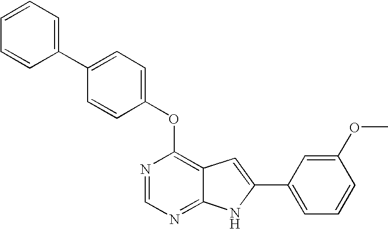 |
| D24 | 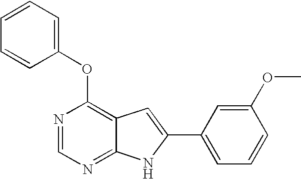 |

Compound E1

### 4-[4-(3,5-Dichloro-phenoxy)-7H-pyrrolo[2,3-d]pyrimidin-6-yl]-phenol; LC-MS: 373 (M⁺+H)

Compound E1 was synthesized by the following procedure:

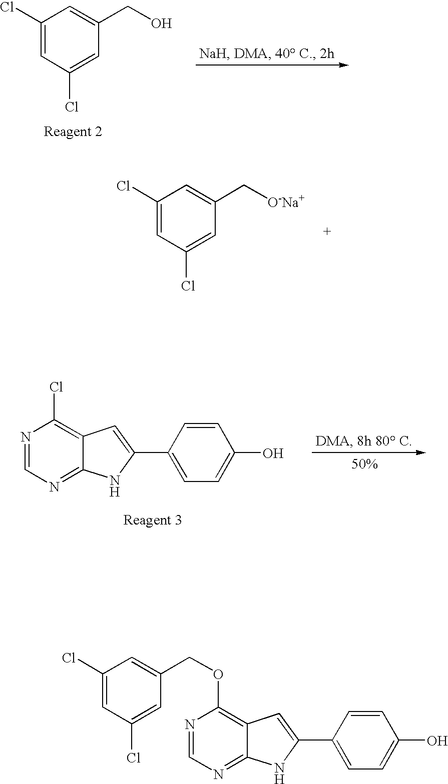

To a 1 mM solution of reagent 2 was added 1.5 eq of NaH in DMA and stirred at 40° C. for 2 hours. The solution was cooled and 0.95 eq Reagent 3 in 0.5 ml DMA was added and the reaction heated at 80° C. for 8 hours. The reaction was cooled to room temperature and purified by reverse phase HPLC (Water:Acetonitirlie solvent system) to obtain 2 mg (45%) of compound E1.

Compounds E2 through E4 were synthesized in a manner analogous to Compound E1 using similar starting materials and reagents. The structures are shown below in Table E:

TABLE E

| | CHEMICAL |
|---|---|
| NO. | STRUCTURE |
| E1 | 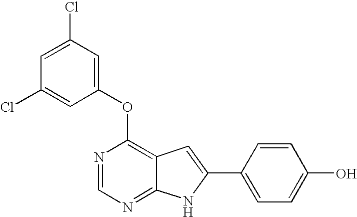 |
| E2 | 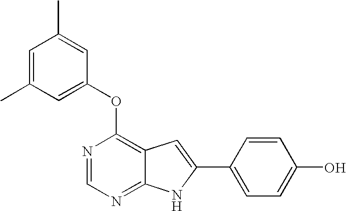 |
| E3 | 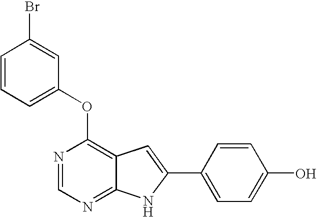 |
| E4 | 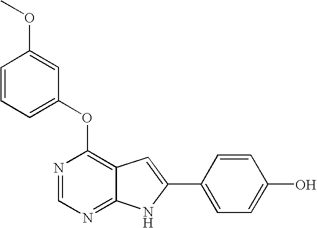 |

### 4-(2-Chloro-phenylsulfanyl)-7H-pyrrolo[2,3-d]pyrimidine; LC-MS: 263 (M⁺+H)

Compound F1 was synthesized by the following procedure:

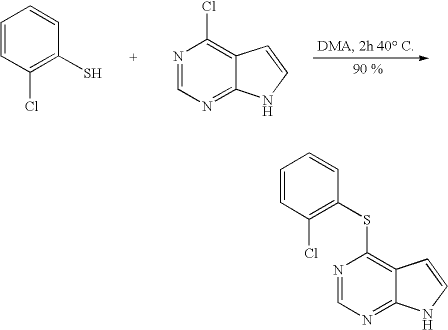

To a 1 mM solution of 2-chlorothiophenol in DMA was added 0.95 eq of 4-Chloro-7H-pyrrolo[2,3-d]pyrimidine in 0.5 ml DMA and the reaction heated at 40° C. for 2 hours. The reaction was cooled to room temperature and purified by reverse phase HPLC (Water:Acetonitrile solvent system) to obtain 8 mg (90%) of compound F1.

Compounds F2 through F10 were synthesized in a manner analogous to Compound F1 using similar starting materials and reagents. The structures are shown below in Table F:

TABLE F

| | CHEMICAL |
|---|---|
| NO. | STRUCTURE |
| F1 | 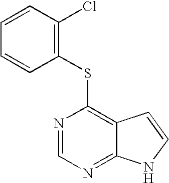 |
| F2 | 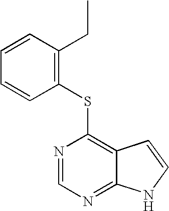 |
| F3 | 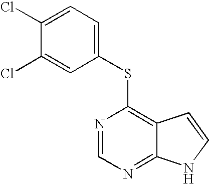 |
| F4 | 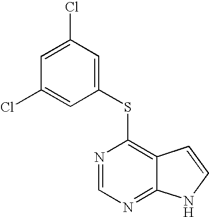 |
| F5 | 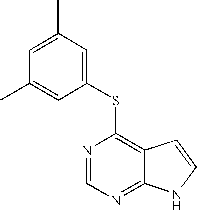 |
| F6 | 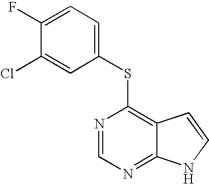 |
| F7 | 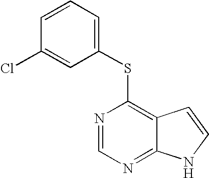 |
| F8 | 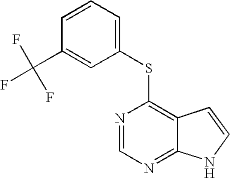 |
| F9 | 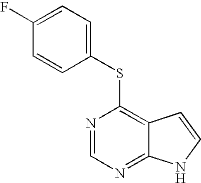 |
| F10 | 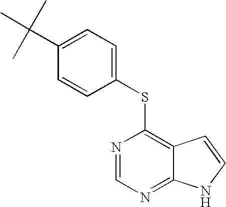 |

Compound G1

### 4-Benzylsulfanyl-7H-pyrrolo[2,3-d]pyrimidine; LC-MS: 243 (M⁺+H)

Compound G1 was synthesized by the following procedure:

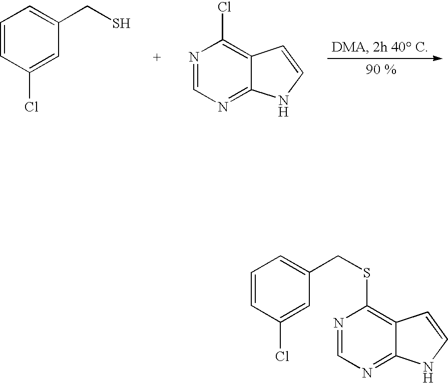

To a 1 mM solution of 3-chlorothiobenzyl alcohol in DMA was added 0.95 eq of 4-Chloro-7H-pyrrolo[2,3-d]pyrimidine in 0.5 ml DMA and the reaction heated at 40° C. for 2 hours. The reaction was cooled to room temperature and purified by reverse phase HPLC (Water:Acetonitrile solvent system) to obtain 9 mg (90%) of compound G1.

Compound H1

### 4-(4-Methoxy-benzylsulfanyl)-6-(4-methoxy-phenyl)-7H-pyrrolo[2,3-d]pyrimidine; LC-MS: 379 (M⁺+H)

Compound H1 was synthesized by the following procedure:

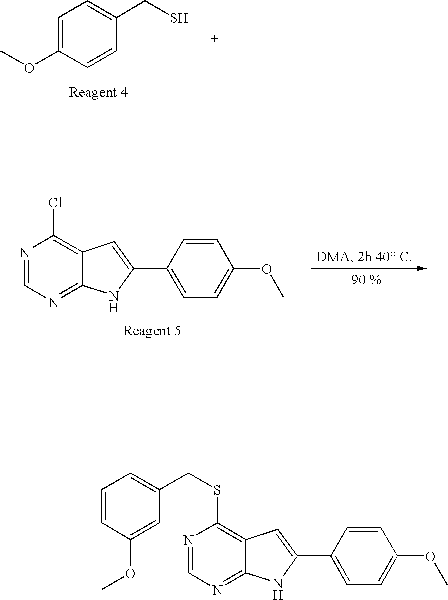

To a 1 mM solution of reagent 4 in DMA was added 0.95 eq of reagent 5 in 0.5 ml DMA and the reaction heated at 40° C. for 2 hours. The reaction was cooled to room temperature and purified by reverse phase HPLC (Water:Acetonitrile solvent system) to obtain 9 mg (90%) of compound H1.

Compounds H2 through H7 were synthesized in a manner analogous to Compound H1 using similar starting materials and reagents. The structures are shown below in Table H:

TABLE H

| | CHEMICAL |
|---|---|
| NO. | STRUCTURE |
| H1 | 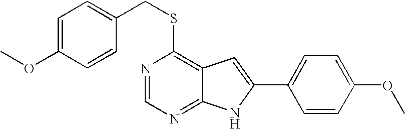 |
| H2 | 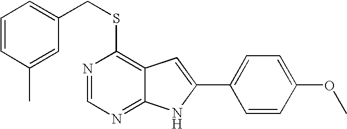 |
| H3 | 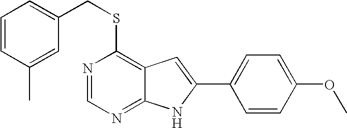 |
| H4 | 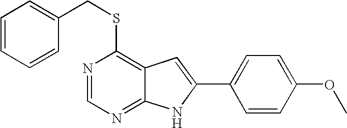 |
| H5 | 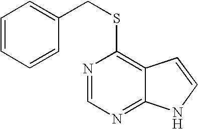 |
| H6 | 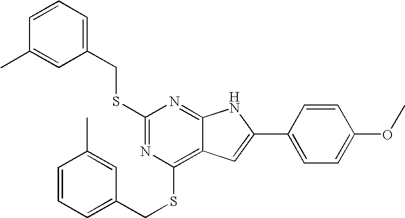 |
| H7 | 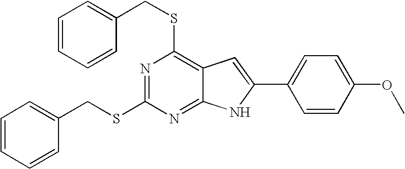 |

Compound I1

### 2,4-Bis-(4-methoxy-benzylsulfanyl)-6-(4-methoxy-phenyl)-7H-pyrrolo[2,3-d]pyrimidine; LC-MS: 531 (M⁺+H)

Compound I1 was synthesized by the following procedure:

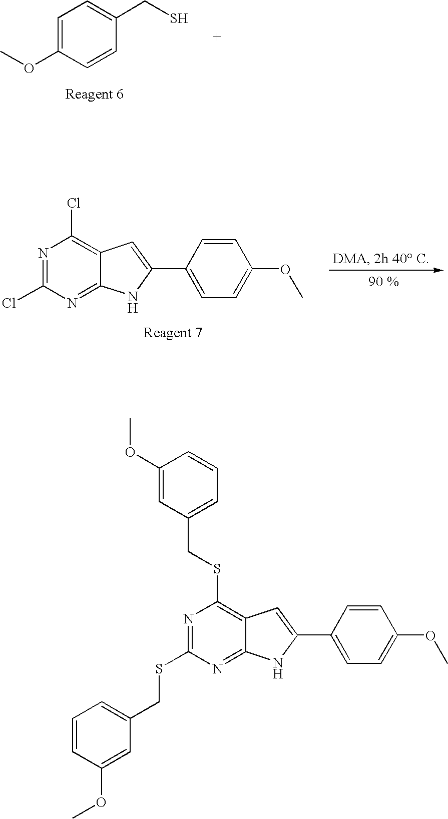

To a 1 mM solution of reagent 6 in DMA was added 0.95 eq of reagent 7 in 0.5 ml DMA and the reaction heated at 40° C. for 2 hours. The reaction was cooled to room temperature and purified by reverse phase HPLC (Water:Acetonitrile solvent system) to obtain 9 mg (90%) of compound I1.

Compounds I2 through I19 were synthesized in a manner analogous to Compound I1 using similar starting materials and reagents. The structures are shown below in Table I:

TABLE I

| | CHEMICAL |
|---|---|
| NO. | STRUCTURE |
| I1 | 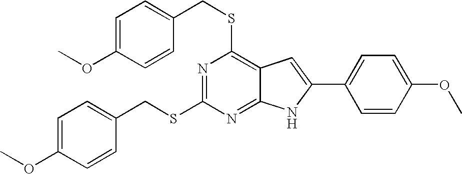 |
| I2 | 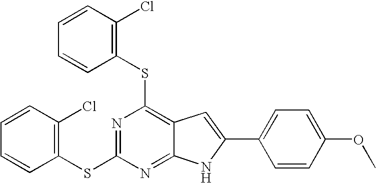 |
| I3 | 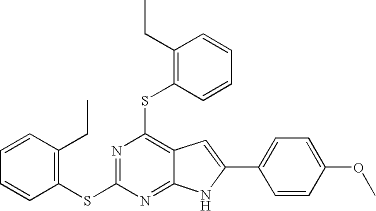 |
| I4 | 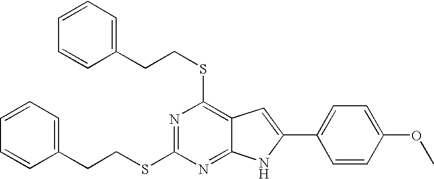 |
| I5 | 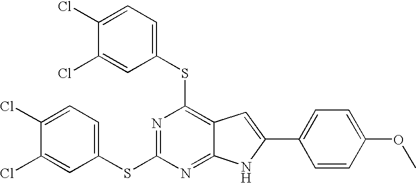 |
| I6 | 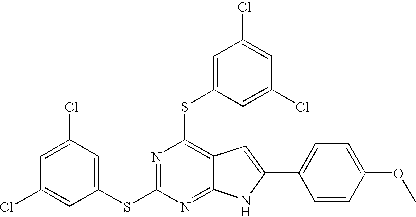 |
| I7 | 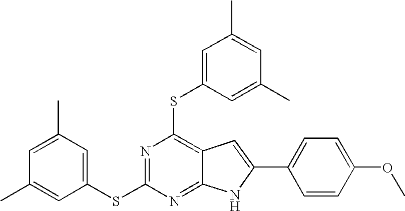 |
| I8 | 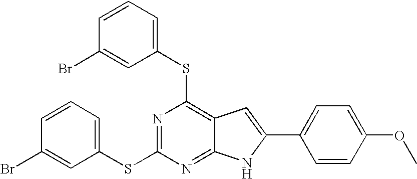 |
| I9 | 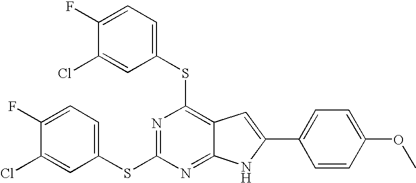 |
| I10 | 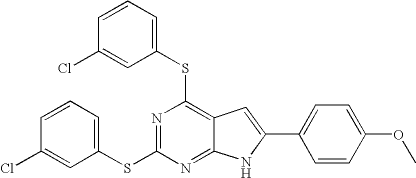 |
| I11 | 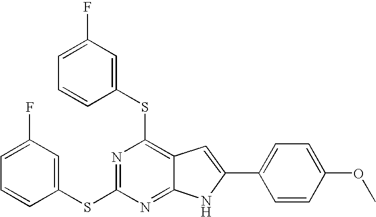 |
| I12 | 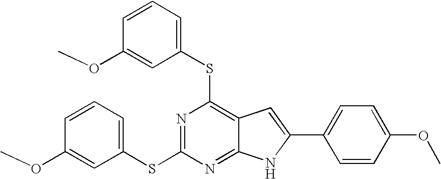 |
| I13 | 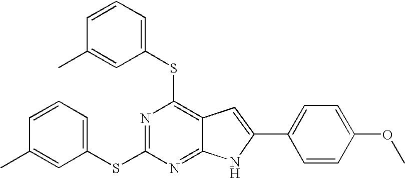 |
| I14 | 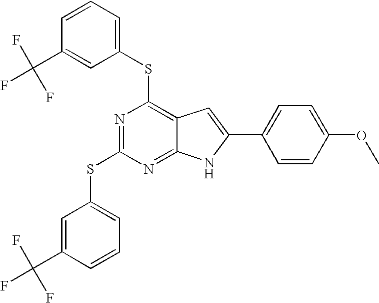 |
| I15 | 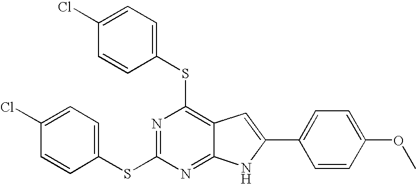 |
| I16 | 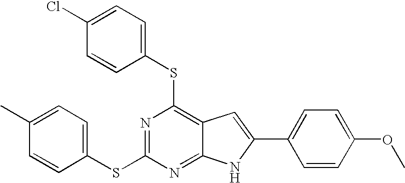 |
| I17 | 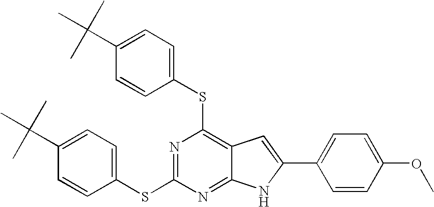 |
| I18 | 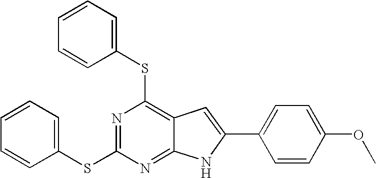 |
| I19 | 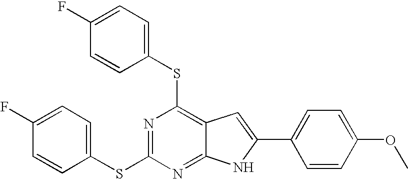 |

Compound J1

### 4-(2-Chloro-phenylsulfanyl)-6-(4-methoxy-phenyl)-7H-pyrrolo[2,3-d]pyrimidine; LC-MS: 369 (M⁺+H)

Compound J1 was synthesized by the following procedure:

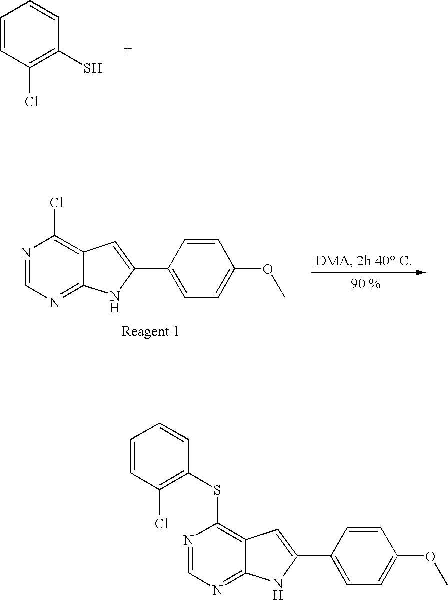

To a 1 mM solution of 2-chlorothiophenol in DMA was added 0.95 eq of reagent 1 in 0.5 ml DMA and the reaction heated at 40° C. for 2 hours. The reaction was then cooled to room temperature and purified by reverse phase HPLC (Water:Acetonitrile solvent system) to obtain 8 mg (90%) of compound I1.

Compounds J2 through J37 were synthesized in a manner analogous to Compound J1 using similar starting materials and reagents. The structures are shown below in Table J:

TABLE J

| | CHEMICAL |
|---|---|
| NO. | STRUCTURE |
| J1 | 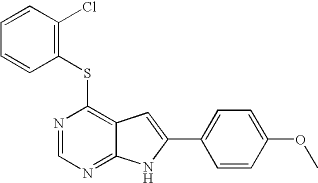 |
| J2 | 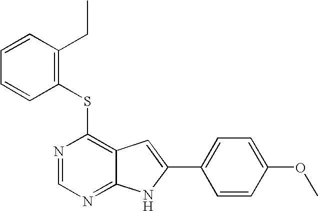 |
| J3 | 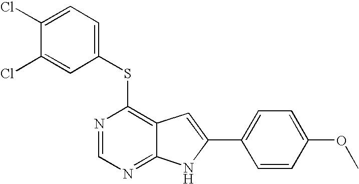 |
| J4 | 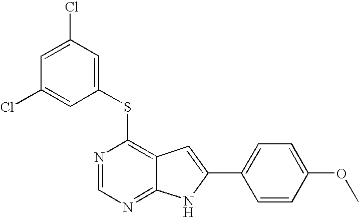 |
| J5 | 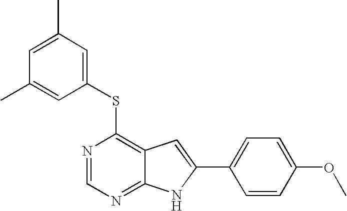 |
| J6 | 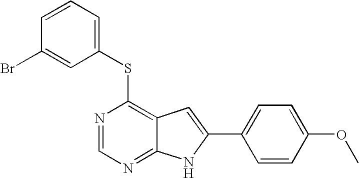 |
| J7 | 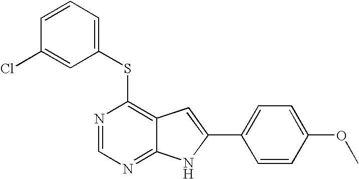 |
| J8 | 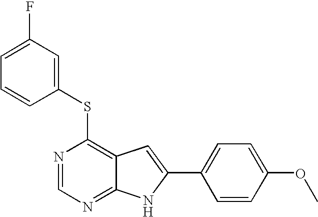 |
| J9 | 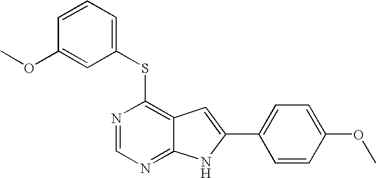 |
| J10 | 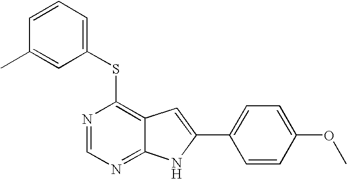 |
| J11 | 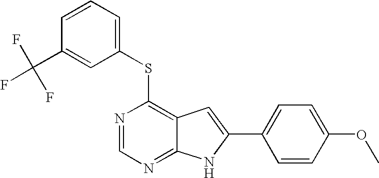 |
| J12 | 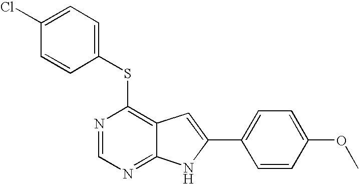 |
| J13 | 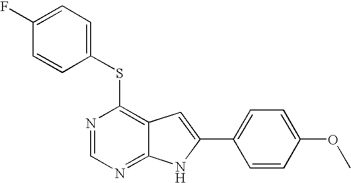 |
| J14 | 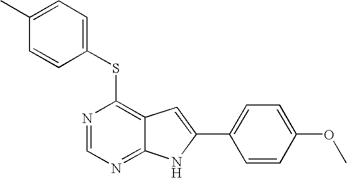 |
| J15 | 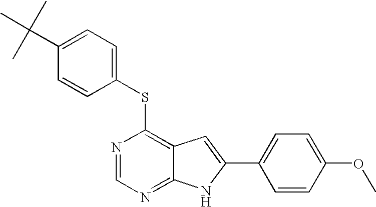 |
| J16 | 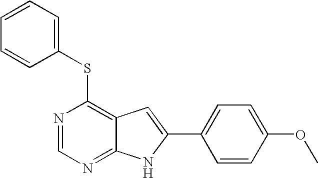 |
| J17 | 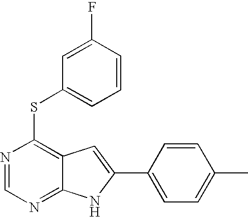 |
| J18 | 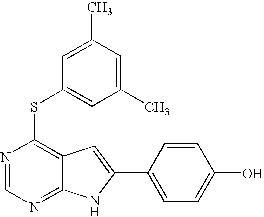 |
| J19 | 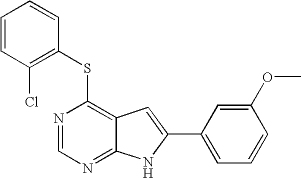 |
| J20 | 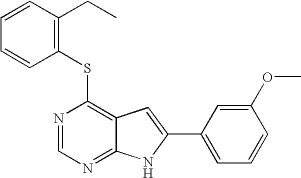 |
| J21 | 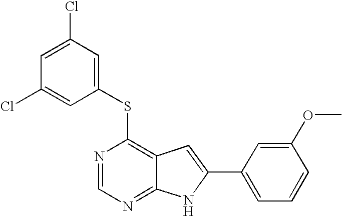 |
| J22 | 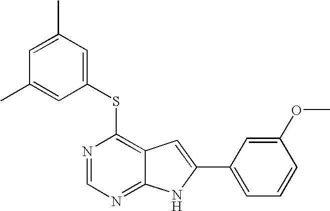 |
| J23 | 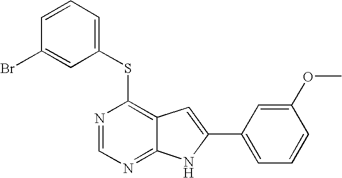 |
| J24 | 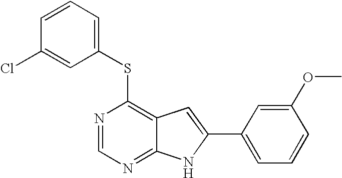 |
| J25 | 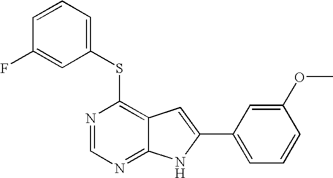 |
| J26 | 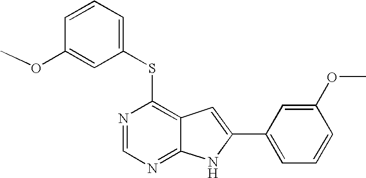 |
| J27 | 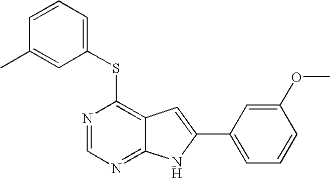 |
| J28 | 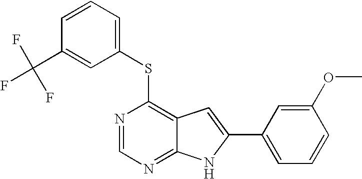 |
| J29 | 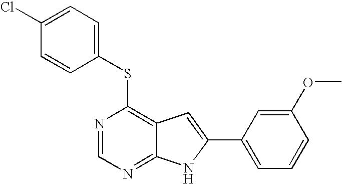 |
| J30 | 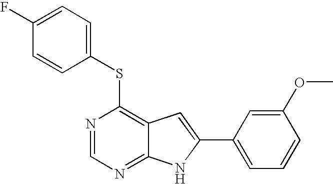 |
| J31 | 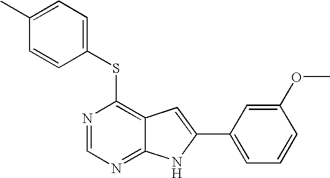 |
| J32 | 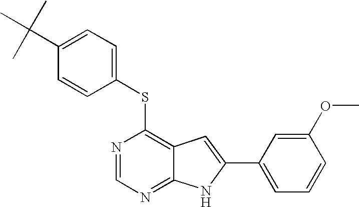 |
| J33 | 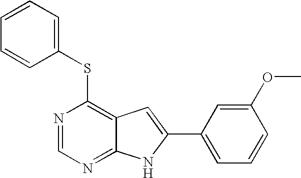 |
| J34 | 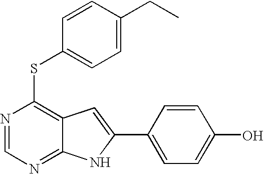 |
| J35 | 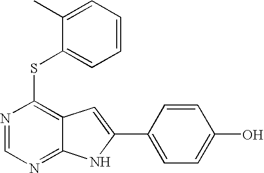 |
| J36 | 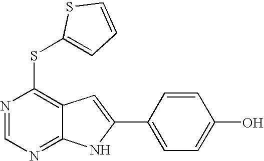 |
| J37 | 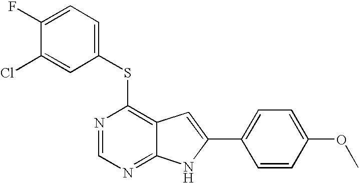 |

Compound K1

### 1-(4-Methoxy-benzylsulfanyl)-9H-2,4,9-triaza-fluorene; LC-MS: 322 (M⁺+H)

Compound K1 was synthesized by the following procedure:

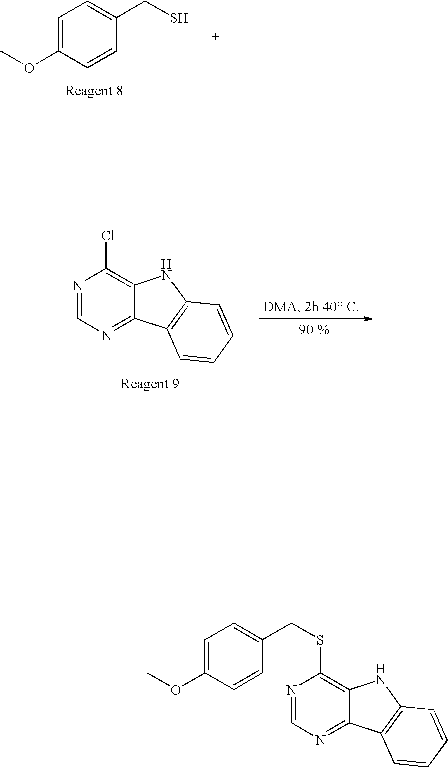

To a 1 mM solution of reagent 8 in DMA was added 0.95 eq of reagent 9 in 0.5 ml DMA and the reaction heated at 40° C. for 2 hours. The reaction was cooled to room temperature and purified by reverse phase HPLC (Water:Acetonitrile solvent system) to obtain 9 mg (90%) of compound K1.

Compounds K2 through K19 were synthesized in a manner analogous to Compound K1 using similar starting materials and reagents. The structures are shown below in Table K:

TABLE K

| | CHEMICAL |
|---|---|
| NO. | STRUCTURE |
| K1 | 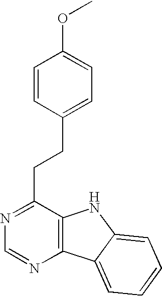 |
| K2 | 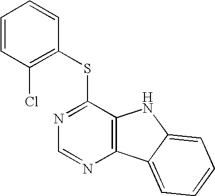 |
| K3 | 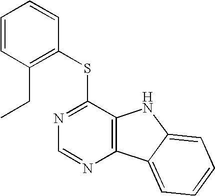 |
| K4 | 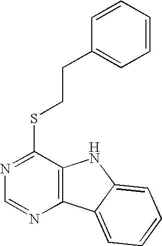 |
| K5 | 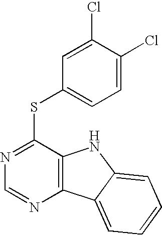 |
| K6 | 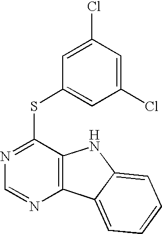 |
| K7 | 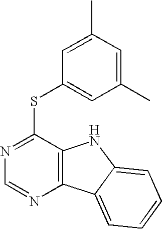 |
| K8 | 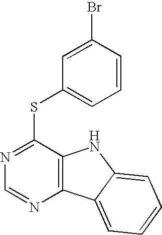 |
| K9 | 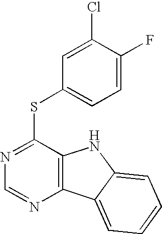 |
| K10 | 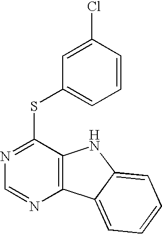 |
| K11 | 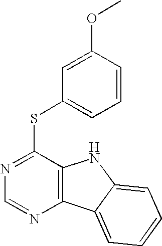 |
| K12 | 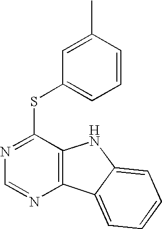 |
| K13 | 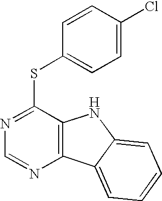 |
| K14 | 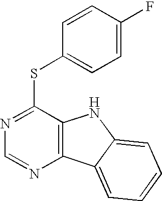 |
| K15 | 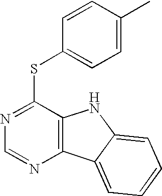 |
| K16 | 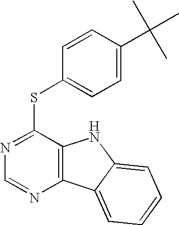 |
| K17 | 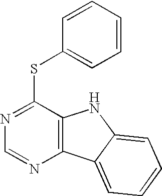 |
| K18 | 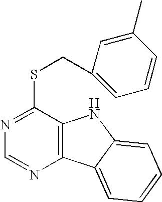 |
| K19 | 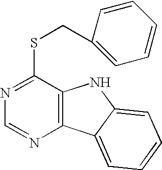 |

Compound L1

### 4-(4-Phenethylsulfanyl-7H-pyrrolo[2,3-d]pyrimidin-6-yl)-phenol; LC-MS: 349 (M⁺+H)

Compound L1 was synthesized by the following procedure:

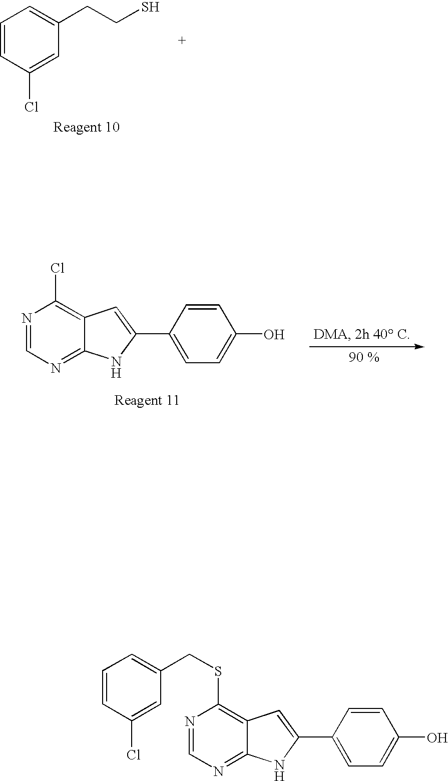

To a 1 mM solution of reagent 10 in DMA was added 0.95 eq of reagent 11 in 0.5 ml DMA and the reaction heated at 40° C. for 2 hours. The reaction was cooled to room temperature and purified by reverse phase HPLC (Water:Acetonitrile solvent system) to obtain 9 mg (90%) of compound L1.

Compounds L1 through L11 were synthesized in a manner analogous to Compound L1 using similar starting materials and reagents. The structures are shown below in Table L:

TABLE L

| | CHEMICAL |
|---|---|
| NO. | STRUCTURE |
| L1 | 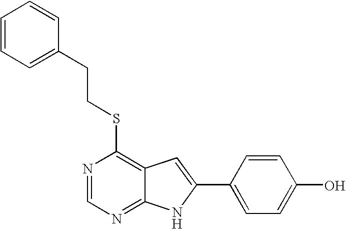 |
| L2 | 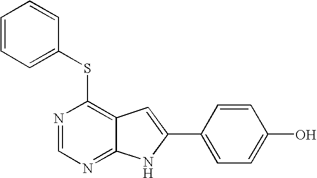 |
| L3 | 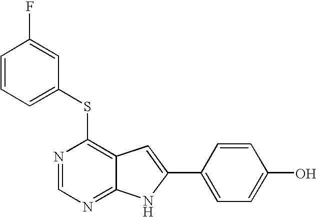 |
| L4 | 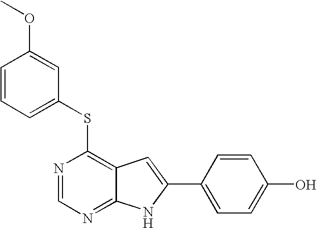 |
| L5 | 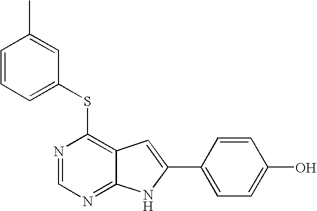 |
| L6 | 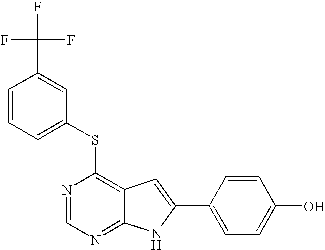 |
| L7 | 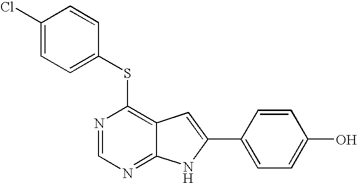 |
| L8 | 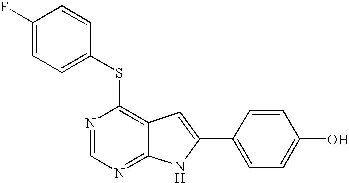 |
| L9 | 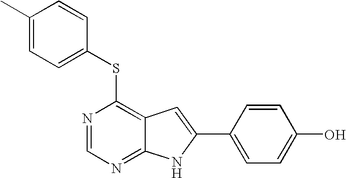 |
| L10 | 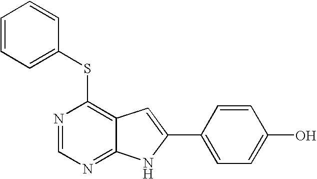 |
| L11 | 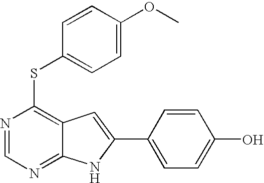 |

Compound M1

### 6-(2-Chloro-phenylsulfanyl)-9H-purine; LC-MS: 264 (M⁺+H)

Compound M1 was synthesized by the following procedure:

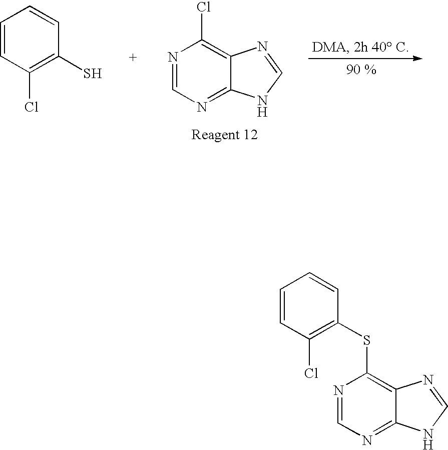

To a 1 mM solution of 2-chlorothiophenol in DMA was added 0.95 eq of reagent 12 in 0.5 ml DMA and the reaction heated at 40° C. for 2 hours. The reaction was cooled to room temperature and purified by reverse phase HPLC (Water:Acetonitrile solvent system) to obtain 8 mg (90%) of compound M1.

Compounds M2 through M18 were synthesized in a manner analogous to Compound M1 using similar starting materials and reagents. The structures are shown below in Table M:

TABLE M

| | CHEMICAL |
|---|---|
| NO. | STRUCTURE |
| M1 | 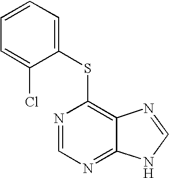 |
| M2 | 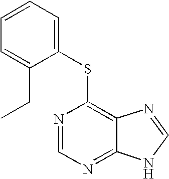 |
| M3 | 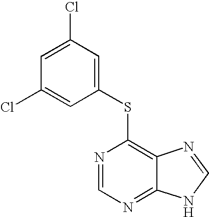 |
| M4 | 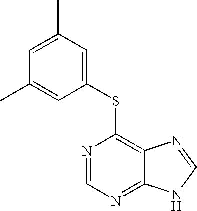 |
| M5 | 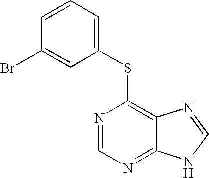 |
| M6 | 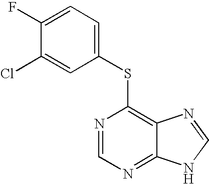 |
| M7 | 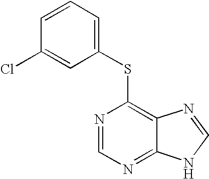 |
| M8 | 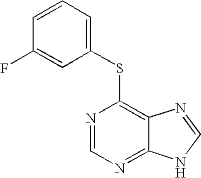 |
| M9 | 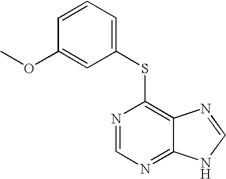 |
| M10 | 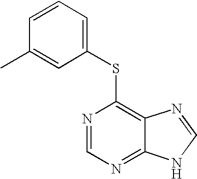 |
| M11 | 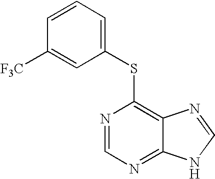 |
| M12 | 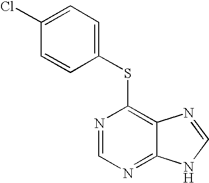 |
| M13 | 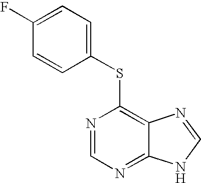 |
| M14 | 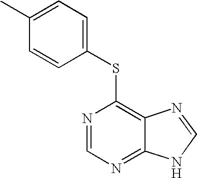 |
| M15 | 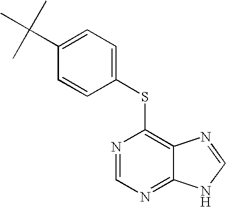 |
| M16 | 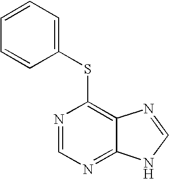 |
| M17 | 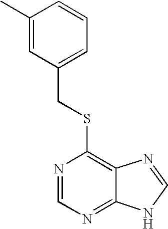 |
| M18 | 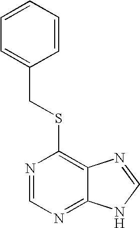 |

Compound N1

### 4-(2-Chloro-phenylsulfanyl)-5,6-diphenyl-furo[2,3-d]pyrimidine; LC-MS: 416 (M⁺+H)

Compound N1 was synthesized by the following procedure:

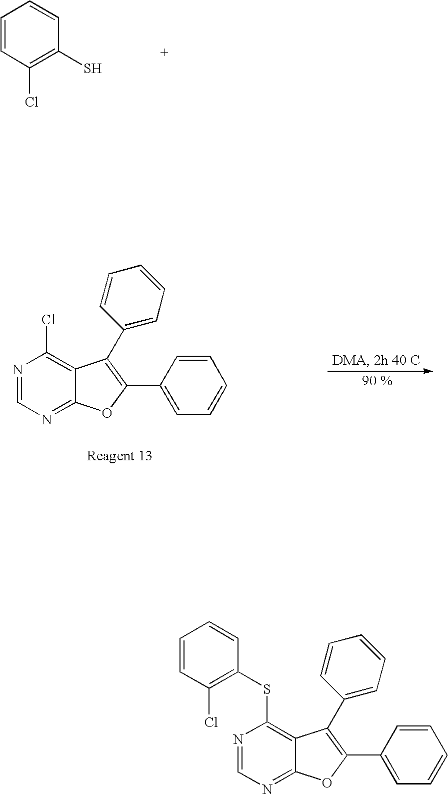

To a 1 mM solution of 2-chlorothiophenol in DMA was added 0.95 eq of reagent 13 in 0.5 ml DMA and the reaction heated at 40° C. for 2 hours. The reaction was cooled to room temperature and purified by reverse phase HPLC (Water:Acetonitrile solvent system) to obtain 8 mg (90%) of compound N1.

Compounds N2 through N18 were synthesized in a manner analogous to Compound N1 using similar starting materials and reagents. The structures are shown below in Table N:

TABLE N

| | CHEMICAL |
|---|---|
| NO. | STRUCTURE |
| N1 | 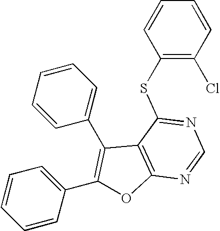 |
| N2 | 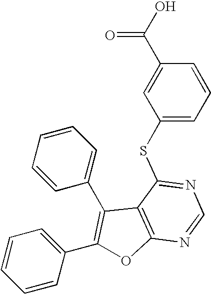 |
| N3 | 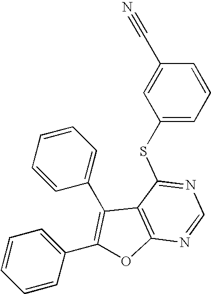 |
| N4 | 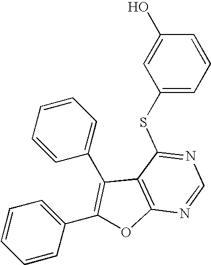 |
| N5 | 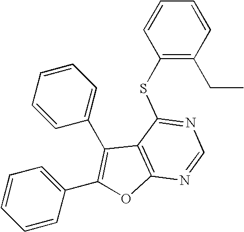 |
| N6 | 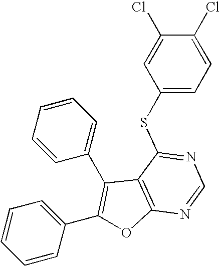 |
| N7 | 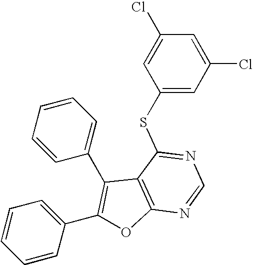 |
| N8 | 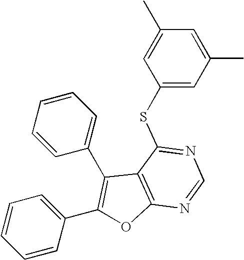 |
| N9 | 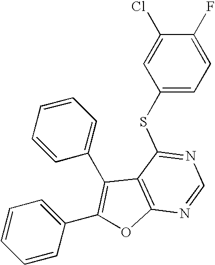 |
| N10 | 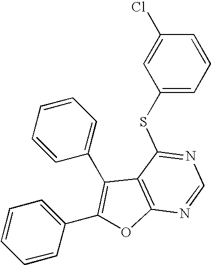 |
| N11 | 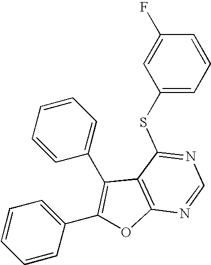 |
| N12 | 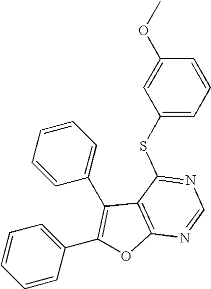 |
| N13 | 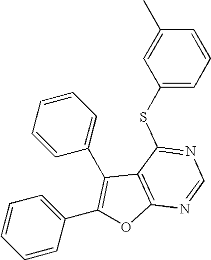 |
| N14 | 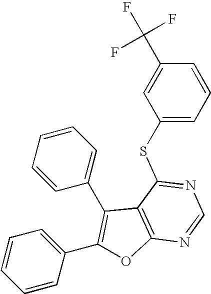 |
| N15 | 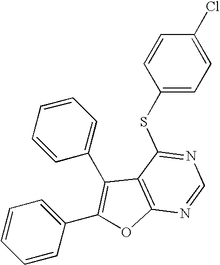 |
| N16 | 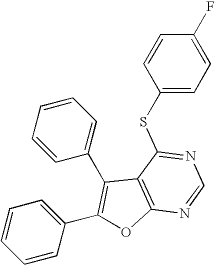 |
| N17 | 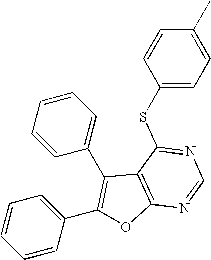 |
| N18 | 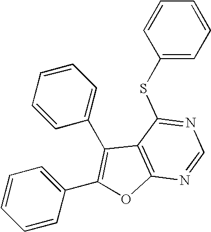 |

Compound O1

### 4-(7-Methyl-4-phenylsulfanyl-7H-pyrrolo[2,3-d]pyrimidin-6-yl)-phenol; LC-MS: 335 (M⁺+H)

Compound O1 was synthesized by the following procedure:

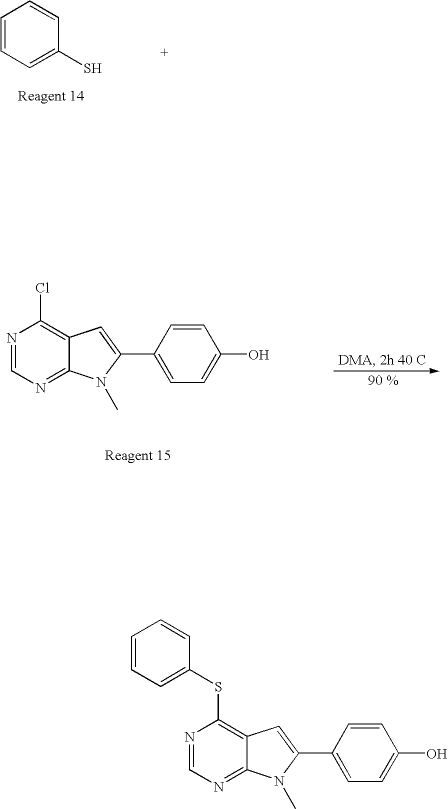

To a 1 mM solution of reagent 14 in DMA was added 0.95 eq of reagent 15 in 0.5 ml DMA and the reaction heated at 40° C. for 2 hours. The reaction was cooled to room temperature and purified by reverse phase HPLC (Water:Acetonitrile solvent system) to obtain 8 mg (90%) of compound O1.

Compounds O2 through O28 were synthesized in a manner analogous to Compound O1 using similar starting materials and reagents. The structures are shown below in Table O:

TABLE O

| | CHEMICAL |
|---|---|
| NO. | STRUCTURE |
| O1 | 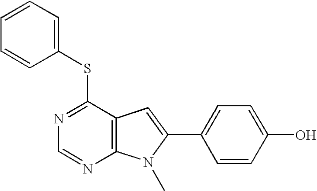 |
| O2 | 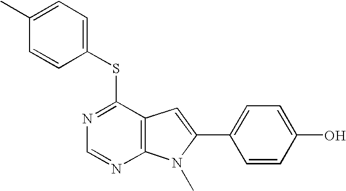 |
| O3 | 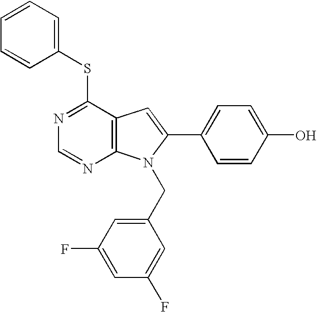 |
| O4 | 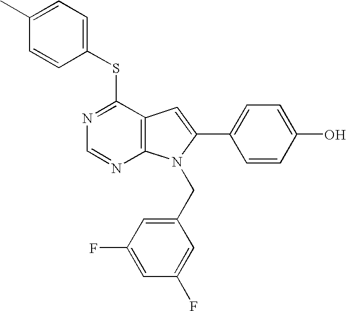 |
| O5 | 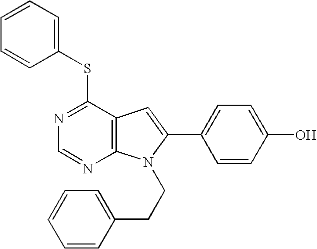 |
| O6 | 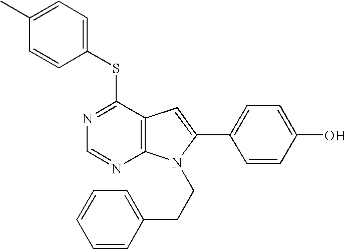 |
| O7 | 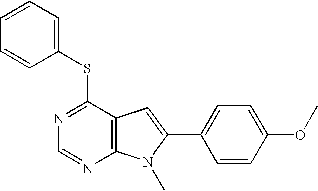 |
| O8 | 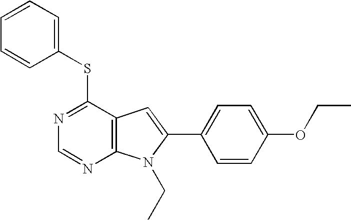 |
| O9 | 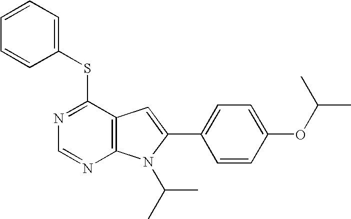 |
| O10 | 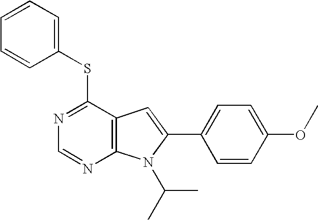 |
| O11 | 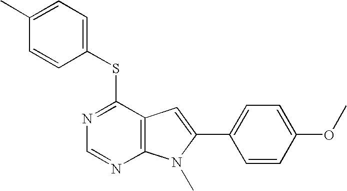 |
| O12 | 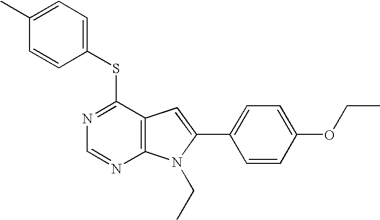 |
| O13 | 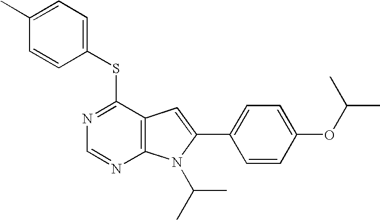 |
| O14 | 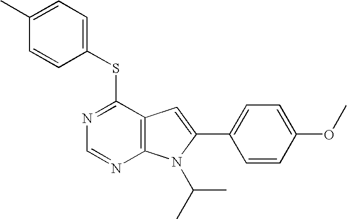 |
| O15 | 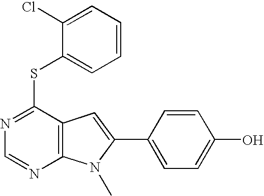 |
| O16 | 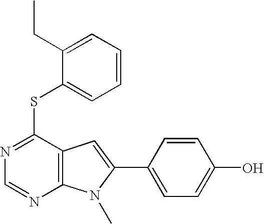 |
| O17 | 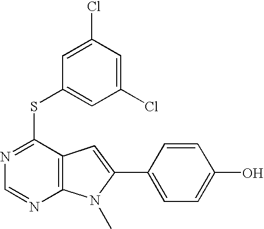 |
| O18 | 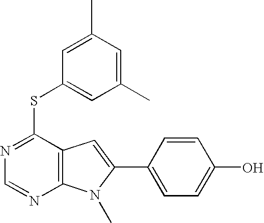 |
| O19 | 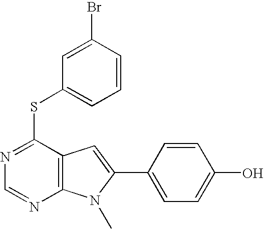 |
| O20 | 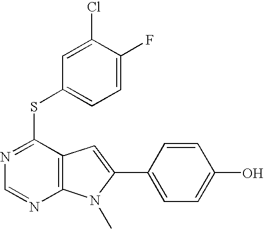 |
| O21 | 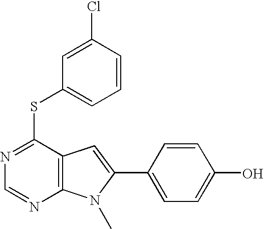 |
| O22 | 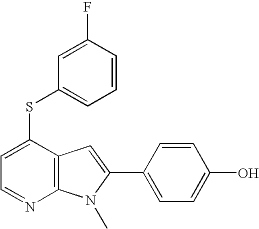 |
| O23 | 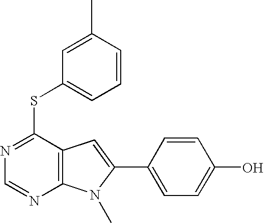 |
| O24 | 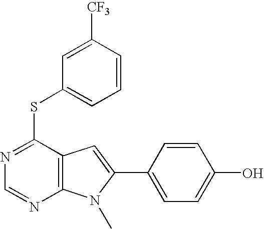 |
| O25 | 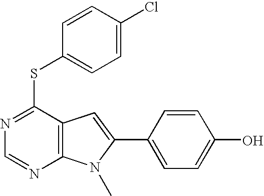 |
| O26 | 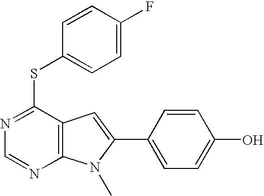 |
| O27 | 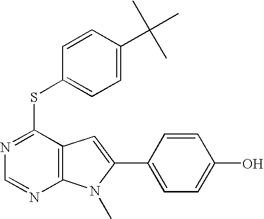 |
| O28 | 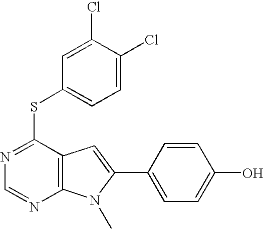 |

Compound P1

### 4-(7-Methyl-2,4-bis-phenylsulfanyl-7H-pyrrolo[2,3-d]pyrimidin-6-yl)-phenol; LC-MS: 443 (M⁺+H)

Compound P1 was synthesized by the following procedure:

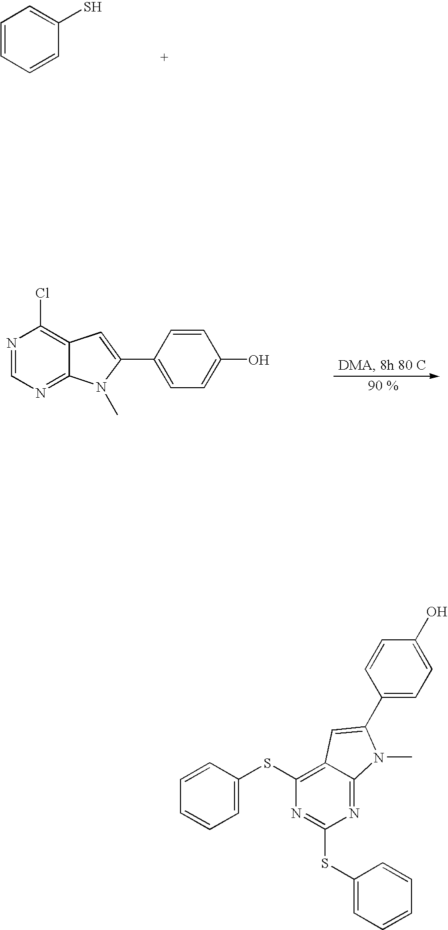

To a 1 mM solution of thiophenol was added 0.95 eq of 4-(4-Chloro-7-methyl-7H-pyrrolo[2,3-d]pyrimidin-6-yl)-phenol in 0.5 ml DMA and the reaction heated at 80° C. for 8 hours. The reaction was cooled to room temperature and purified by reverse phase HPLC (Water:Acetonitrile solvent system) to obtain 2 mg (50%) of compound P1.

Compounds P2 through P16 were synthesized in a manner analogous to Compound P1 using similar starting materials and reagents. The structures are shown below in Table P:

TABLE P

| | CHEMICAL |
|---|---|
| NO. | STRUCTURE |
| P1 | 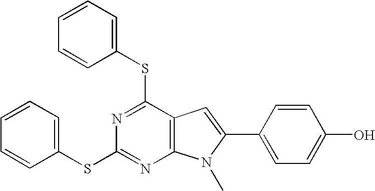 |
| P2 | 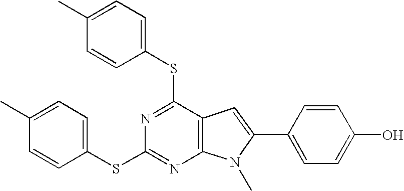 |
| P3 | 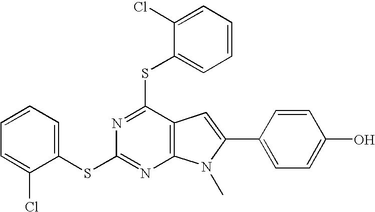 |
| P4 | 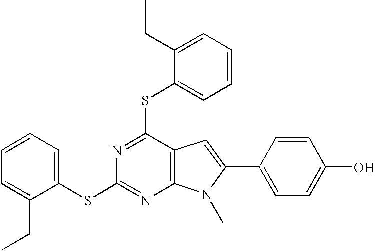 |
| P5 | 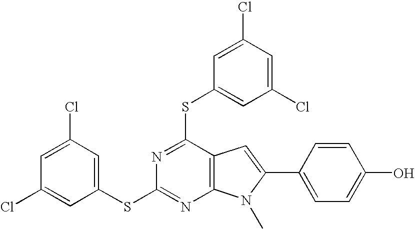 |
| P6 | 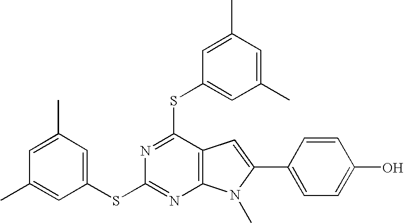 |
| P7 | 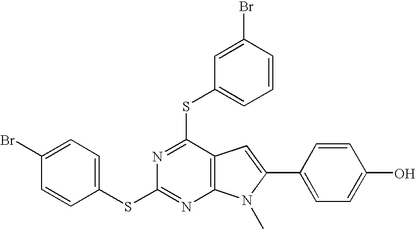 |
| P8 | 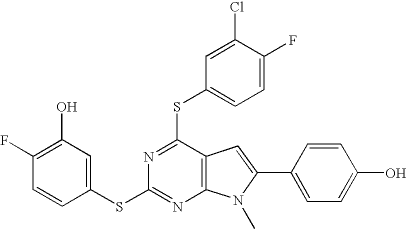 |
| P9 | 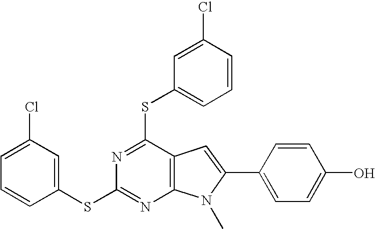 |
| P10 | 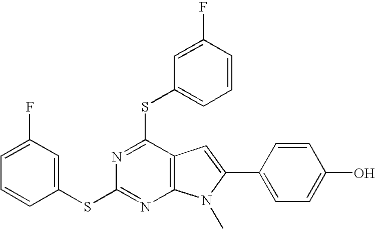 |
| P11 | 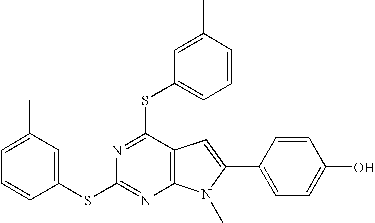 |
| P12 | 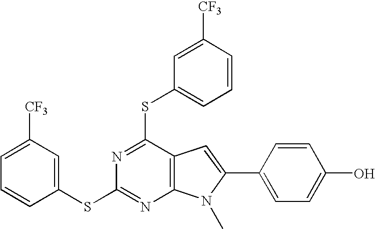 |
| P13 | 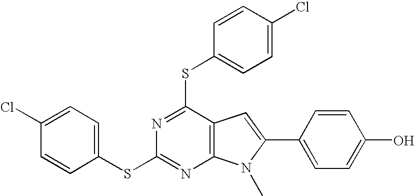 |
| P14 | 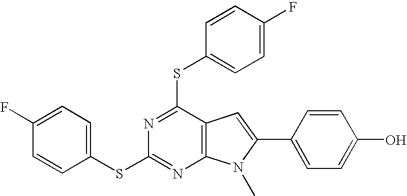 |
| P15 | 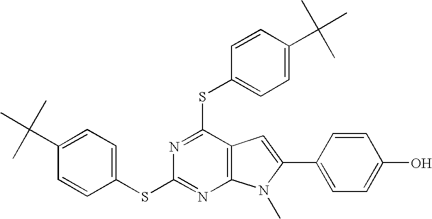 |
| 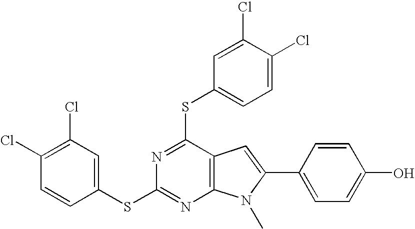 | |

Binding Constant (K_{d}) Measurements for Small-Molecule-Kinase Interactions

Methods for measuring binding affinities for interactions between small molecules and kinases including FLT3, c-KIT, ABL(T3341) [a.k.a. ABL(T3151)], VEGFR-2 (a.k.a. KDR), and EGFR are described in detail in U.S. application Ser. No. 10/873,835, which is incorporated by reference herein in its entirety. The components of the assays include human kinases expressed as fusions to T7 bacteriophage particles and immobilized ligands that bind to the ATP site of the kinases. For the assay, phage-displayed kinases and immobilized ATP site ligands are combined with the compound to be tested. If the test compound binds the kinase it competes with the immobilized ligand and prevents binding to the solid support. If the compound does not bind the kinase, phage-displayed proteins are free to bind to the solid support through the interaction between the kinase and the immobilized ligand. The results are read out by quantitating the amount of fusion protein bound to the solid support, which is accomplished by either traditional phage plaque assays or by quantitative PCR (qPCR) using the phage genome as a template. To determine the affinity of the interactions between a test molecule and a kinase, the amount of phage-displayed kinase bound to the solid support is quantitated as a function of test compound concentration. The concentration of test molecule that reduces the number of phage bound to the solid support by 50% is equal to the Kd for the interaction between the kinase and the test molecule. Typically, data are collected for twelve concentrations of test compound and, the resultant binding curve is fit to a non-cooperative binding isotherm to calculate K_{d}.

Described in the exemplary assays below is data from binding with varying kinases. Binding values are reported as follows “+” for representative compounds exhibiting a binding dissociation constant (Kd) of 10,000 nM or higher; “++” for representative compounds exhibiting a Kd of 1,000 nM to 10,000 nM; “+++” for representative compounds exhibiting a Kd of 100 nM to 1,000 nM; and “++++” for representative compounds exhibiting a Kd of less than 100 nM. The term “ND” represents non-determined values.

### The Affinity of the Compounds for EGFR

To measure the Kd values, the T7 phage displaying human EGFR were incubated with an atorvastatin-coated affinity matrix in the presence of various concentrations of a soluble (non-immobilized) compounds provided herein, as described in detail herein. Soluble compounds that bind EGFR prevent binding of EGFR phage to the affinity matrix; hence, fewer phage are recovered in the phage eluate in the presence of an effective competitor than in the absence of an effective competitor. The Kd for the interaction between the soluble compound (competitor) molecule and EGFR is equal to the concentration of soluble competitor molecule that causes a 50% reduction in the number of phage recovered in the eluate compared to a control sample lacking soluble competitor.

### EGFR Autophosphorylation Inhibition Assay

Tyrosine 1173 is a major autophosphorylation site resulting from activation of EGFR by epidermal growth factor (EGF). To determine the capacity of a compound to inhibit this phosphorylation activity of EGFR upon itself, the following methodology was used: 4×10⁴ A431 cells/well in a 96-well culture plate or 3.6×10⁵ A549 cells/well in a 24-well culture plate were cultured overnight at 37° C. in 5% CO₂ in low serum culture medium (DMEM supplemented with 0.5% fetal calf serum, 4.500 mg/L glucose and 100 units/ml penicillin-streptomycin). After 16 hours, the cells were pre-incubated in eight serial 3-fold dilutions of test compound (3.3 μM-0.0017 μM) in addition to vehicle control (final concentration on DMSO vehicle was 1%) for two hours. Cells were stimulated by the addition of 5 ng/ml of EGF for five minutes. Cells were then washed with cold phosphate buffered saline (PBS), and incubated for 30 minutes at 4° C. in lysis buffer. Subsequently, the samples were centrifuged at 6000× RCF for 15 minutes, and the level of phosphorylation of EGFR tyrosine 1173 was measured using a sandwich enzyme-linked immunosorbent assay following the manufacturer's recommended protocols (Biosource, Camarillo, Calif.). Total EGFR levels were also measured in the same manner to control for protein level differences. The reported values are those concentrations of compound required to inhibit EGF-induced phosphorylation of tyrosine 1173 by 50%.

### A431 Proliferation Inhibition Assay

To examine the ability of a compound to inhibit proliferation of the A431 cell line, the following methodology was used: 2000 cells/well in a 96-well culture plate were cultured overnight at 37° C. in 5% CO₂ in low serum medium (DMEM supplemented with 0.5% fetal calf serum, 4.500 mg/L glucose and 100 units/ml penicillin-streptomycin). After 16 hours, medium was replaced with low serum medium containing 10 serial 3-fold dilutions of compound plus a vehicle control (final concentration of DMSO vehicle was 1%), and the cells were incubated at 37° C. in 5% CO₂ for 72 hours. Relative cell number was using 3-(4,5-dimethylthiazol-2-yl)-5(3-carboxymethoxyphenyl)-2-(4-sulfophenyl)-2H-tetrazolium) (MTS) following the manufacturer's recommended protocol (Promega, Madison, Wis.). The reported values are those concentrations of compound required to inhibit cell proliferation by 50%.

Data for some of the compounds is provided below.

Binding of wildtype-EGFR

| Compound | Kd for EGFR(DKIN) |
|---|---|
| No. | Binding (nM) |
| F3 | +++ |
| J3 | +++ |
| J37 | ++++ |
| J7 | ++++ |
| J8 | ++++ |
| J9 | +++ |
| J10 | +++ |
| J11 | +++ |
| J13 | ++++ |
| J14 | ++++ |
| J16 | +++ |
| D3 | +++ |
| D5 | ++++ |
| D8 | ++++ |
| C3 | + |
| C4 | +++ |
| J17 | +++ |
| J18 | +++ |
| K5 | ++++ |
| K8 | ++++ |
| K10 | ++++ |
| K12 | +++ |
| K13 | ++++ |
| K14 | ++++ |
| K15 | ++++ |
| K17 | ++++ |
| D18 | +++ |
| D19 | ++++ |
| D20 | +++ |
| D21 | +++ |
| J19 | ++++ |
| J20 | ++++ |
| J21 | + |
| J22 | +++ |
| J23 | ++++ |
| J24 | ++++ |
| J25 | ++++ |
| J26 | +++ |
| J27 | +++ |
| J28 | ++++ |
| J30 | ++++ |
| J31 | ++++ |
| J32 | ++ |
| J33 | ++++ |
| E1 | +++ |
| E2 | ++++ |
| E3 | ++++ |
| E4 | +++ |
| L1 | ++ |
| L2 | ++++ |
| L3 | ++++ |
| L4 | +++ |
| L5 | ++++ |
| L7 | ++++ |
| L8 | ++++ |
| L9 | ++++ |
| N4 | +++ |
| N12 | +++ |
| L11 | +++ |

Binding Data of mutant-EGFR- Kd Expressed in nM

| | Compound | Compound | Compound |
|---|---|---|---|
| | No. | No. | No. |
| Assay | K5 | E3 | L2 |
| (L861Q) | +++ | ++++ | ++++ |
| (G719C) | ++++ | ++++ | ++++ |
| (G719S) | ++++ | ++++ | ++++ |
| (S752-1759del) | ++++ | ++++ | ++++ |
| (L747-T751del, Sins) | ++++ | ++++ | ++++ |
| (L747-T752del, P753S) | ++++ | ++++ | ++++ |
| (L747-E749del, A750P) | ++++ | ++++ | ++++ |
| (L858R) | ++++ | ++++ | ++++ |
| (E746-A750del) | ++++ | ++++ | ++++ |

Cell Assay Data for EGFR Phosphorylation
in Epidermoid Carcinoma Cell Line A431

| Compound | |
|---|---|
| No. | IC50 (nM) |
| J37 | +++ |
| J7 | ++ |
| J8 | ++ |
| J13 | ++ |
| J14 | ++ |
| D5 | +++ |
| K5 | +++ |
| K8 | +++ |
| K10 | +++ |
| K13 | +++ |
| K14 | +++ |
| K15 | +++ |
| D19 | +++ |
| J19 | ++ |
| J20 | ++ |
| J23 | +++ |
| J24 | +++ |
| J25 | +++ |
| J27 | +++ |
| J28 | ++ |
| J30 | ++ |
| J31 | ++ |
| J33 | +++ |
| E1 | +++ |
| E3 | ++++ |
| L2 | ++++ |
| L3 | ++++ |
| L5 | ++++ |
| L7 | +++ |
| L8 | ++++ |
| L9 | +++ |

Cell Assay Data for EGFR Phosphorylation
in Lung Cancer Cell Line A459

| Compound | |
|---|---|
| No. | IC50 (nM) |
| J7 | ++ |
| J8 | ++ |
| J14 | ++ |
| K5 | +++ |
| K10 | +++ |
| K14 | +++ |
| D19 | ++ |
| J19 | ++ |
| J23 | +++ |
| J24 | +++ |
| J25 | ++ |
| J27 | ++ |
| J30 | ++ |
| J31 | + |
| J33 | ++ |
| E3 | +++ |
| L2 | +++ |
| L3 | ++ |
| L5 | ++ |
| L7 | ++ |
| L8 | ++ |
| L9 | ++ |

All references cited herein, including patents, patent applications, and publications, are herby incorporated by reference in their entireties, whether previously specifically incorporated or not.

Having now fully described compounds and methods provided herein, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

1. A method for treating a disease comprising administering to a subject in need thereof an effective amount of an epidermal growth factor receptor modulating compound corresponding to Formula (I): 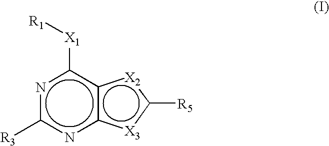
wherein:
a. X₁ is S or O;
b. each of X₂ and X₃ is independently N, O, S, NR₄, or CR₆;
c. R₁ is —(CHR₁ₐ)_{z}—R_{1b}, where
i. each R₁ₐ is independently H, (C₁-C₄)alkyl, F, (C₁-C₄)fluoroalkyl, (C₁-C₄)alkoxy, —C(O)OH, —C(O)—NH₂, —C(O)—(C₁-C₄)alkyl, —C(O)—(C₁-C₄)fluoralkyl, —C(O)—(C₁-C₄)alkylamine, —(C₁-C₄)alkylamine, —(C₁-C₄)dialkylamine, or —C(O)—(C₁-C₄)alkoxy,
ii. z is 0, 1, 2, or 3, and
iii. R_{1b} is 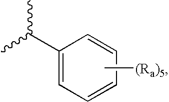
where each Rₐ is independently H, halogen, substituted or unsubstituted alkyl, substituted or unsubstituted alkoxy, —CN, -L₁-OH, -L₁-NH₂, -L₁-(C₁-C₄)alkyl, -L₁-(C₃-C₆)cycloalkyl, -L₁-(C₁-C₄)fluoroalkyl, -L₁-(C₁-C₄)alkoxy, -L₁-(C₁-C₄)alkylamine, -L₁-(C₁-C₄)dialkylamine and -L₁-phenyl, wherein L₁ is a bond, —C(O)—, or —S(O)₂—; or
R_{1b} is H, —(C₁-C₄)alkyl, an optionally substituted —(C₃-C₆)cycloalkyl, —(C₁-C₄)fluoroalkyl, or an optionally substituted 5-membered or 6-membered unsaturated heterocycle;
d. R₃ is H or L₃-(CHR₃ₐ)ₓ—R_{3b}, where
i. L₃ is a bond, NH, O, or S,
ii. R₃ₐ is H, (C₁-C₄)alkyl, F, (C₁-C₄)fluoroalkyl, (C₁-C₄)alkoxy, —(C₁-C₄)alkylamine, or —(C₁-C₄)dialkylamine,
iii. x is 0, 1, 2, or 3, and
iv. R_{3b} is phenyl, optionally substituted with 1-2 substituents independently selected from the group consisting of halogen, —(C₁-C₄)alkyl, —(C₁-C₄)fluoroalkyl, —(C₁-C₄)alkoxy, —(C₁-C₄)alkylamine, and —(C₁-C₄)dialkylamine;
e. R₄ is H or —(CHR₄ₐ)_{y}—R_{4b}, where
i. R₄ₐ is H, (C₁-C₄)alkyl, F, (C₁-C₄)fluoroalkyl, (C₁-C₄)alkoxy, —(C₁-C₄)alkylamine, or —(C₁-C₄)dialkylamine;
ii. y is 0, 1, 2, or 3, and
iii. R_{4b} is substituted or unsubstituted alkyl, substituted or unsubstituted cycloalkyl, substituted or unsubstituted phenyl, or substituted or unsubstituted 5-membered or 6-membered unsaturated heterocycle; or
R₄ and R₅, taken together, form a 5- or 6-membered heterocyclic aromatic ring structure, optionally substituted with 1-2 moieties independently selected from the group consisting of halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, and —(C₁-C₄)dialkylamine; or
when X₂is NR₄ and X₃ is CR₆, R₁ and R₄, taken together, form a 5- or 6-membered aromatic heterocycle optionally substituted with 1-2 moieties independently selected from the group consisting of halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, and —(C₁-C₄)dialkylamine; or
f. R₅ is H or 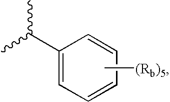
where each R_{b} is independently H, halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, —(C₁-C₄)dialkylamine, —C(O)OH, —C(O)—NH₂, —C(O)—(C₁-C₄)alkyl, —C(O)—(C₁-C₄)fluoralkyl, —C(O)—(C₁-C₄)alkylamine, or —C(O)—(C₁-C₄)alkoxy; and
g. R₆ is H, heteroaryl, or phenyl, wherein the phenyl and the heteroaryl are optionally substituted with 1-2 moieties independently selected from the group consisting of halogen, —(C₁-C₄)alkyl, —(C₁-C₄)fluoroalkyl, —(C₁-C₄)alkoxy, —(C₁-C₄)alkylamine, and —(C₁-C₄)dialkylamine; or
R₆ and R₅, taken together, form an aromatic carbocycle or heterocycle optionally substituted with 1-2 moieties independently selected from the group consisting of halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, and —(C₁-C₄)dialkylamine, or
when X₂ is CR₆ and X₃ is NR₄, R₆ and R₁, taken together, form a 5- or 6-membered aromatic heterocycle optionally substituted with 1-2 moieties independently selected from the group consisting of halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, and —(C₁-C₄)dialkylamine;
or a pharmaceutically acceptable salt, pharmaceutcaly acceptable N-oxide, pharmaceutically acitve metabolite, pharmaceutically acceptable prodrug, or pharmaceutically acceptable solvate thereof.

2. The method of claim 1, wherein said compound corresponds to Formula (Ia-O):

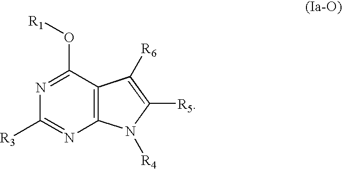

3. The method of claim 1, wherein said compound corresponds to Formula (Ia-S):

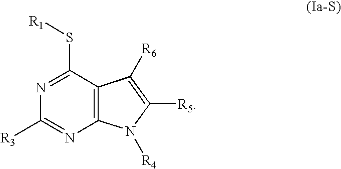

4. The method of claim 1, wherein said compound corresponds to Formula (IIb-O):

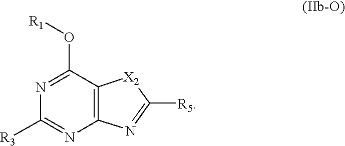

5. The method of claim 1, wherein said compound corresponds to Formula (IIb-S):

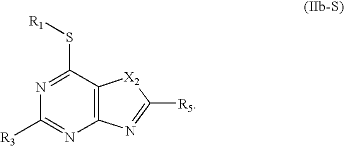

6. The method of claim 1, wherein said compound corresponds to Formula (IIIa-O):

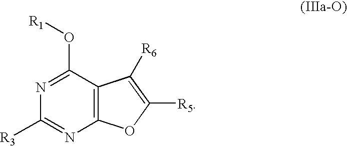

7. The method of claim 1, wherein said compound corresponds to Formula (IIIa-S):

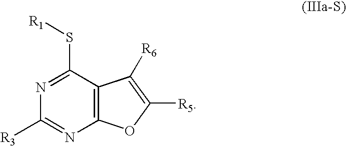

8. The method of claim 1, wherein said compound corresponds to Formula (IIIb-O):

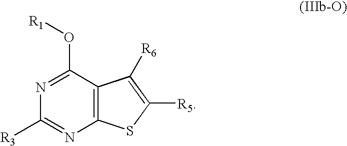

9. The method of claim 1, wherein said compound corresponds to Formula (IIb-S):

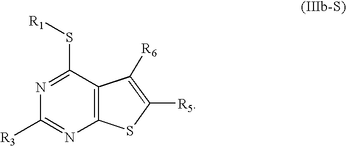

10. The method of claim 1, wherein said compound corresponds to Formula (A1):

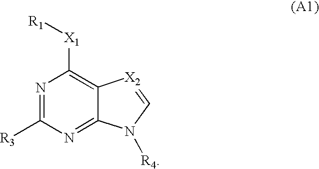

11. The method of claim 1, wherein said compound corresponds to Formula (A):

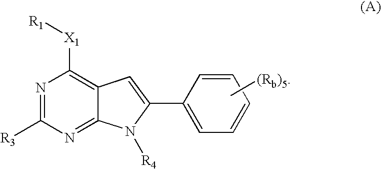

12. The method of claim 11, wherein said compound corresponds to Formula (B):

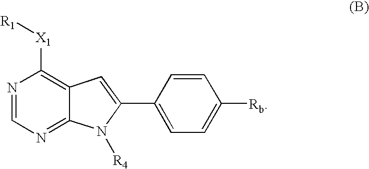

13. The method of claim 11, wherein said compound corresponds to Formula (C):

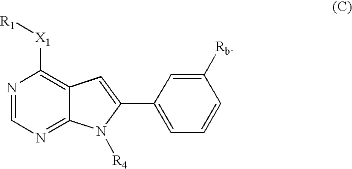

14. The method of claim 1, wherein said compound corresponds to Formula (D):

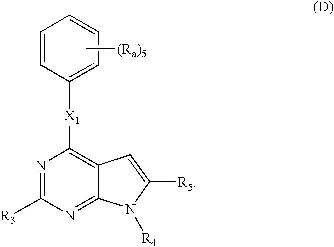

15. The compound of claim 14, corresponding to Formula (E):

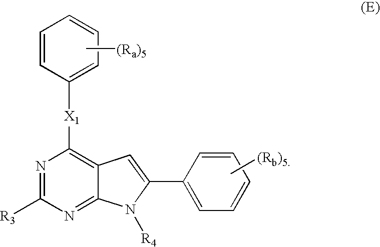

16. The method of claim 1, wherein said compound corresponds to Formula (IV): 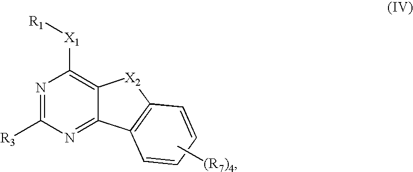
wherein
X₂ is O, S, or NR₄; and
each R₇ is independently selected from the group consisting of H, halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, —(C₁-C₄)dialkylamine, —C(O)OH, —C(O)—NH₂, —C(O)—(C₁-C₄)alkyl, —C(O)—(C₁-C₄)fluoralkyl, —C(O)—(C₁-C₄)alkylamine, and —C(O)—(C₁-C₄)alkoxy.

17. The method of claim 16, wherein said compound corresponds to Formula (F):

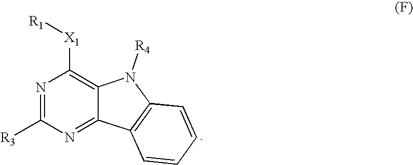

18. The method of claim 17, wherein said compound corresponds to Formula (G):

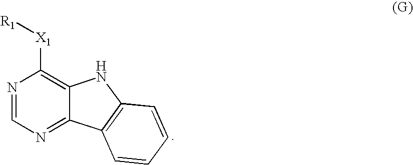

19. The method of claim 18, wherein said compound corresponds to Formula (H):

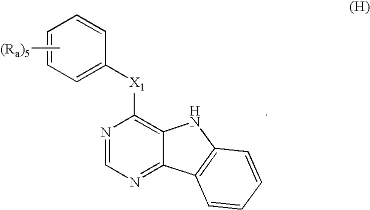

20. A method for treating a disease comprising administering to a subject in need thereof an effective amount of an EGFR kinase modulating compound corresponding to Formula (I): 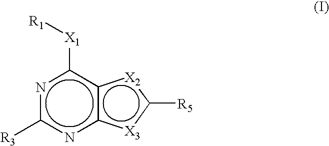
wherein:
a. X₁ is S or O;
b. each of X₂ and X₃ is independently N, O, S, NR₄, or CR₆;
c. R₁ is —(CHR₁ₐ)_{z}—R_{1b}, where
i. each R₁ₐ is independently H, (C₁-C₄)alkyl, F, (C₁-C₄)fluoroalkyl, (C₁-C₄)alkoxy, —C(O)OH, —C(O)—NH₂, —C(O)—(C₁-C₄)alkyl, —C(O)—(C₁-C₄)fluoralkyl, —C(O)—(C₁-C₄)alkyl amine, —(C₁-C₄)alkylamine, —(C₁-C₄)dialkylamine, or —C(O)—(C₁-C₄)alkoxy,
ii. z is 0, 1, 2, or 3, and
iii. R_{1b} is 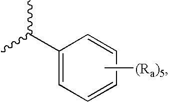
where each Rₐ is independently H, halogen, substituted or unsubstituted alkyl, substituted or unsubstituted alkoxy, —CN, -L₁-OH, -L₁-NH₂, -L₁-(C₁-C₄)alkyl, -L₁-(C₃-C₆)cycloalkyl, -L₁-(C₁-C₄)fluoroalkyl, -L₁-(C₁-C₄)alkoxy, -L₁-(C₁-C₄)alkylamine, -L₁-(C₁-C₄)dialkylamine and -L₁-phenyl, wherein L₁ is a bond, —C(O)—, or —S(O)₂—; or
R_{1b} is H, —(C₁-C₄)alkyl, an optionally substituted —(C₃-C₆)cycloalkyl, —(C₁-C₄)fluoroalkyl, or an optionally substituted 5-membered or 6-membered unsaturated heterocycle;
d. R₃ is H or L₃-(CHR₃ₐ)ₓ—R_{3b}, where
i. L₃ is a bond, NH, O, or S,
ii. R₃ₐ is H, (C₁-C₄)alkyl, F, (C₁-C₄)fluoroalkyl, (C₁-C₄)alkoxy, —(C₁-C₄)alkylamine, or —(C₁-C₄)dialkylamine,
iii. x is 0, 1, 2, or 3, and
iv. R_{3b} is phenyl, optionally substituted with 1-2 substituents independently selected from the group consisting of halogen, —(C₁-C₄)alkyl, —(C₁-C₄)fluoroalkyl, —(C₁-C₄)alkoxy, —(C₁-C₄)alkylamine, and —(C₁-C₄)dialkylamine;
e. R₄ is H or —(CHR₄ₐ)_{y}—R_{4b}, where
i. R₄ₐ is H, (C₁-C₄)alkyl, F, (C₁-C₄)fluoroalkyl, (C₁-C₄)alkoxy, —(C₁-C₄)alkylamine, or —(C₁-C₄)dialkylamine;
ii. y is 0, 1, 2, or 3, and
iii. R_{4b} is substituted or unsubstituted alkyl, substituted or unsubstituted cycloalkyl, substituted or unsubstituted phenyl, or substituted or unsubstituted 5-membered or 6-membered unsaturated heterocycle; or
R₄ and R₅, taken together, form a 5- or 6-membered heterocyclic aromatic ring structure, optionally substituted with 1-2 moieties independently selected from the group consisting of halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, and —(C₁-C₄)dialkylamine; or
when X₂ is NR₄ and X₃ is CR₆, R₁ and R₄, taken together, form a 5- or 6-membered aromatic heterocycle optionally substituted with 1-2 moieties independently selected from the group consisting of halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, and —(C₁-C₄)dialkylamine; or
f. R₅ is H or 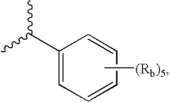
where each R_{b} is independently H, halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, —(C₁-C₄)dialkylamine, —C(O)OH, —C(O)—NH₂, —C(O)—(C₁-C₄)alkyl, —C(O)—(C₁-C₄)fluoralkyl, —C(O)—(C₁-C₄)alkylamine, or —C(O)—(C₁-C₄)alkoxy; and
g. R₆ is H, heteroaryl, or phenyl, wherein the phenyl and the heteroaryl are optionally substituted with 1-2 moieties independently selected from the group consisting of halogen, —(C₁-C₄)alkyl, —(C₁-C₄)fluoroalkyl, —(C₁-C₄)alkoxy, —(C₁-C₄)alkylamine, and —(C₁-C₄)dialkylamine; or
R₆ and R₅, taken together, form an aromatic carbocycle or heterocycle optionally substituted with 1-2 moieties independently selected from the group consisting of halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, and —(C₁-C₄)dialkylamine, or
when X₂ is CR₆ and X₃ is NR₄, R₆ and R₁, taken together, form a 5- or 6-membered aromatic heterocycle optionally substituted with 1-2 moieties independently selected from the group consisting of halogen, —CN, —OH, —NH₂, —(C₁-C₄)alkyl, —(C₃-C₆)cycloalkyl, —(C₁-C₄)fluoroalkyl, —(C₁-C₄)alkoxy, —(C₁-C₄)alkylamine, and —(C₁-C₄)dialkylamine;
or a pharmaceutically acceptable salt, pharmaceutcaly acceptable N-oxide, pharmaceutically acitve metabolite, pharmaceutically acceptable prodrug, or pharmaceutically acceptable solvate thereof.

21. The method of claim 20, wherein the contacting occurs within a human patient, wherein the human patient has an EGFR-mediated disease or condition.

22. The method of claim 21, wherein the effective amount is an amount effective for treating an EGFR-mediated disease or condition within the body of the person.

23. The method of claim 22 wherein the EGFR-mediated disease or condition is selected from the group consisting of blood vessel growth, cancer, benign hyperplasia, keloid formation, and psoriasis.

24. A method for treating a disease comprising administering to a subject in need thereof an effective amount of an epidermal growth factor receptor modulating compound corresponding to: 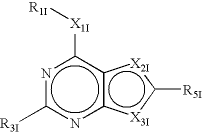
wherein:
a. X_{1I} is or O;
b. each of X_{2I} and X_{3I} is independently N, O, S, NR_{4I}, or CR_{6I};
c. R_{1I} is —(CHR_{1aI})_{zI}—R_{1bI}, where
i. each R_{1aI} is independently H, halogen or a substituted or unsubstituted moiety selected from alkyl, haloalkyl, heteroalkyl, cycloalkyl, heterocycloalkyl, alkenyl, alkynyl, alkoxy, alkylamine, dialkylamine, —C(O)OH, —C(O)NH₂, —C(O)-alkyl, —C(O)-haloalkyl, —C(O)-alkylamine, and —C(O)-alkoxy,
ii. z_{I} is 0, 1, 2, 3, or 4 and
iii. R_{1bI} is 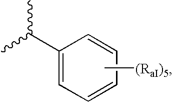
where each R_{aI} is independently H, halogen, —CN, —OH, or a substituted or unsubstituted moiety selected from the group consisting of alkyl, alkoxy, haloalkyl, alkenyl, alkynyl, heteroalkyl, -L₁-OH, -L₁-NH₂, -L₁-alkyl, -L₁-cycloalkyl, -L₁-haloalkyl, -L₁-alkoxy, -L₁-alkylamine, -L₁-dialkylamine and -L₁-phenyl, wherein L₁ is a bond, —C(O)—, or —S(O)₂—; or
R_{1bI} is H, alkyl, or a substituted or unsubstituted moiety selected from cycloalkyl, haloalkyl, and heterocycle;
d. R_{3I} is H or L_{3I}-(CHR_{3aI})ₓ—R_{3bI}, where
i. L_{3I} is a bond, NH, O, or S,
ii. R_{3aI} is H, alkyl, halogen, haloalkyl, alkoxy, alkylamine, or dialkylamine,
iii. x_{I} is 0, 1, 2, 3, or 4 and
iv. R_{3bI} is H or substituted or unsubstituted aryl or heteroaryl group;
e. R_{4I} is H or —(CHR_{4aI})_{yI}—R_{4bI}, where
i. R_{4aI} is H, alkyl, halogen, haloalkyl, alkoxy, alkylamine, or dialkylamine;
ii. y_{I} is 0, 1, 2, 3, or 4 and
iii. R_{4bI} is a substituted or unsubstituted moiety selected from alkyl, cycloalkyl, heterocycloalkyl, aryl, and heteroaryl; or
R_{4I} and R_{5I}, taken together, form a substituted or unsubstitued heteroaryl moiety; or
when X_{1I} is NR_{4I} and X_{2I} is CR_{6I}, R_{1I} and R_{4I}, taken together, form a substituted or
unsubstituted heterocycle; or
f. R_{5I} is H or 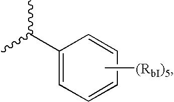
where each R_{bI} is independently H, halogen, —CN, —OH, —NH₂, or a substituted or unsubstituted moiety selected from alkyl, cycloalkyl, haloalkyl, alkoxy, alkylamine, dialkylamine, —C(O)OH, —C(O)—NH₂, —C(O)-alkyl, —C(O)-haloalkyl, —C(O)-alkylamine, and —C(O)-alkoxy; and
g. R_{6I} is H, substituted or unsubstituted heteroaryl, or substituted or unsubstituted aryl; or
R_{6I} and R_{5I}, taken together, form a substituted or unsubstituted aryl or heteroaryl moiety, or
when X_{1I} is CR_{6I} and X_{2I} is NR_{4I}, R_{6I} and R_{1I}, taken together, form a substituted or unsubstituted heterocycle;
or a pharmaceutically acceptable salt, pharmaceutcaly acceptable N-oxide, pharmaceutically acitve metabolite, pharmaceutically acceptable prodrug, or pharmaceutically acceptable solvate thereof.

25. The method of claim 24, wherein the disease is selected from the group consisting of blood vessel growth, cancer, benign hyperplasia, keloid formation, and psoriasis.
